(19) United States
(12) Patent Application Publication
(10) Pub. No.: US 2013/0040304 A1
(43) Pub. Date: Feb. 14, 2013

(54) Compositions and Methods for Engineering Cells

(71) Applicants: UMA LAKSHMIPATHY, CARLSBAD, CA (US); BHASKAR THYAGARAJAN, CARLSBAD, CA (US); JONATHAN CHESNUT, CARLSBAD, CA (US); VASILIKI ANEST, SAN DIEGO, CA (US); ROBERT BENNETT, ENCINITAS, CA (US); PAULINE LIEU, SAN DIEGO, CA (US); GEORGE HANSON, EUGENE, OR (US); DAVID THOMPSON, MONONA, WI (US); LUCAS CHASE, DEFOREST, WI (US); GARY SHIPLEY, PORTLAND, OR (US); ELIZABETH WILSON, TUALATIN, OR (US)

(72) Inventors: UMA LAKSHMIPATHY, CARLSBAD, CA (US); BHASKAR THYAGARAJAN, CARLSBAD, CA (US); JONATHAN CHESNUT, CARLSBAD, CA (US); VASILIKI ANEST, SAN DIEGO, CA (US); ROBERT BENNETT, ENCINITAS, CA (US); PAULINE LIEU, SAN DIEGO, CA (US); GEORGE HANSON, EUGENE, OR (US); DAVID THOMPSON, MONONA, WI (US); LUCAS CHASE, DEFOREST, WI (US); GARY SHIPLEY, PORTLAND, OR (US); ELIZABETH WILSON, TUALATIN, OR (US)

(73) Assignee: LIFE TECHNOLOGIES CORPORATION, CARLSBAD, CA (US)

(21) Appl. No.: 13/571,141

(22) Filed: Aug. 9, 2012

Related U.S. Application Data
(63) Continuation of application No. 13/540,539, filed on Jul. 2, 2012, now abandoned.
Application No. 13/540,539 is a continuation of application No. 12/618,700, filed on Nov. 13, 2009, now abandoned.
(60) Provisional application No. 61/115,013, filed on Nov. 14, 2008.

(51) Int. Cl. C12N 15/79; C12Q 1/68; C12Q 1/04; C12N 5/10

(52) U.S. Cl. 435/6.12; 435/320.1; 435/325; 435/366; 435/349; 435/363; 435/354; 435/353; 435/352; 435/419; 435/412; 435/417; 435/34

(57) ABSTRACT

The disclosure relates generally to genetic manipulation of stem and primary cells and to reprogramming of somatic cells, more specifically, human cells. In particular, compositions and methods are disclosed for the generation and maintenance of such engineered cells.

### CROSS REFERENCE

This application is a continuation of U.S. application Ser. No. 13/540,539 filed Jul. 2, 2012, which is a continuation of U.S. application Ser. No. 12/618,700 filed Nov. 13, 2009, and claims priority to U.S. Provisional Application No. 61/115,013 filed Nov. 14, 2008, all of which are herein incorporated by reference in their entirety.

### FIELD OF THE INVENTION

This invention relates generally to genetic manipulation and/or reprogramming of cells. In particular, compositions and methods are provided that can manipulate any cell (e.g., stem cell), which includes embryonic, fetal or progenitor stem cells, or can reprogram somatic cells to a less differentiated state, towards a more pluripotent embryonic stem cell-like state. Stem cells, or stem cell-like cells thus generated may be useful in research, medicine and other related fields.

### SUMMARY OF THE INVENTION

The invention is directed to compositions and methods related to molecular biology. In certain aspects, the invention provides nucleic acid molecules and methods directed to cell engineering.

In a specific aspect, the invention provides, in part, nucleic acid molecules (e.g., isolated nucleic acid molecules) which have one or more (e.g., one, two, three or four) of the following components: (a) an OriP site, (b) a DNA segment encoding EBNA1; (c) one or more (e.g., one, two, three, four, five, six, seven, eight, etc.) recombination sites (e.g., one or more att sites); and/or (c) at least one selectable marker (e.g., at least one positive or negative selectable marker, including at least one positive selectable marker and at least one negative selectable marker).

Although in most instances, the invention refers to the EBNA1 protein of the EBV virus, also encompassed in the invention is any other equivalent episome maintaining protein or proteins derived from other episomal viruses such as adeno-associated virus (AAV), SV40, BSOLV, HIV-1, etc., and the genes encoding these episomal proteins and/or their OriP elements may also be used to generate vectors of the invention.

The invention further comprises two or more nucleic acid molecules which have at least two of the components referred to above (e.g., a mixture of two vectors herein one vector contains an OriP site and the other vector encodes EBNA1 or a cell line which contains a vector having an OriP site, where a cellular chromosome encodes EBNA1). These two or more nucleic acid molecules may be present in the same composition or separated from each other (e.g., in different vectors, or in containers present in a kit).

The invention is directed to an isolated nucleic acid molecule comprising (a) an OriP site and a DNA segment encoding EBNA1; (b) one or more att recombination sites; and (c) a DNA segment encoding at least one selectable marker. In one aspect, the EBNA1 expression may be constitutive or inducible.

The invention is further directed to an isolated nucleic acid molecule comprising one or more expression cassettes, wherein each expression cassette is operably linked to a promoter for expression, and where each expression cassette can be introduced into the nucleic acid molecule using at least one of the one or more att recombination sites.

In all aspects of the invention, the expression cassette may encode for a tissue-specific gene, stem cell marker gene or a developmental gene.

In all aspects of the invention, the stem cell marker gene is selected from a group consisting of Oct4, Sox2, c-Myc and Klf4; Oct3/4, Nanog, SSEA1, and TRA1-80.

In all aspects of the invention, the promoter driving the expression cassette is of a type selected from a group consisting of cell-specific promoters, tissue-specific promoters, stem cell marker promoters, developmental gene promoters, etc. In a further embodiment, the promoter may be a native promoter of mammalian origin, or an engineered promoter, or a cell-specific promoter, or a developmental stage-specific promoter.

In all aspects of the invention, the stem cell marker promoter is selected from a group of promoters consisting of Oct4, Sox2, c-Myc and Klf4; Oct3/4, Nanog, SSEA1, and TRA1-80. In one embodiment, the developmental stage may be either a germ, embryonic, progenitor, fetal, neonatal, or stem cell stage.

In a specific embodiment, the mammal is human.

In all aspects of the invention, the selectable marker may be either a fluorescent protein, a protein that confers antibiotic resistance, or an enzyme.

In a further aspect, the selectable marker is a fluorescent protein.

In all aspects of the invention, the fluorescent protein may be selected from a group consisting of green fluorescent proteins (GFP) and its modified mutants, red fluorescent proteins (RFP) and its modified mutants, etc.

In a specific aspect, the fluorescent protein is GFP.

In a specific embodiment, the cell is a stem cell.

In all aspects of the invention, the selectable marker may be a protein that confers antibiotic resistance.

In a further embodiment, the antibiotic may be selected from a group consisting of tetracycline, neomycin, blasticidin, hygromycin, ampicillin, and puromycin.

In a specific embodiment, the antibiotic is hygromycin.

In a second aspect, the invention is directed to a first isolated nucleic acid molecule comprising: (a) all or part of a viral genome; (b) an OriP site; (c) one or more att recombination sites; (d) optionally, a DNA segment encoding EBNA1; and (e) at least one selectable marker. In a third aspect, the isolated nucleic acid molecule further comprises (e) the WPRE and/or the VSV-G element.

In some aspects, the DNA segment encoding EBNA1 is on the same nucleic acid molecule.

In other aspects, the DNA segment encoding EBNA1 is on a second isolated nucleic acid molecule, and further comprises (a) all or part of a viral genome; (b) an OriP site; (c) one or more att recombination sites; and (d) at least one selectable marker.

In an aspect, the invention is directed to an isolated nucleic acid molecule comprising: (a) all or part of a viral genome; (b) one or more expression cassettes driven by a promoter; (c) at least one selectable marker; and (d) optionally, a DNA segment encoding a WPRE and/or the VSV-G elements.

In one embodiment, the viral genome is from, either, an insect virus, adenovirus, lentivirus, retrovirus, etc.

In a further embodiment, the viral genome is from an insect virus.

In a specific embodiment, the insect virus is a baculovirus.

One aspect of the invention is directed to a cell transduced with one or more nucleic acid molecules defined herein, each carrying at least one expression cassette for reprogramming said cell.

In a further aspect, the cell is a stem cell.

In an embodiment, the cell is an adult somatic cell.

The invention is directed to various uses of the vectors described above. In one aspect, the vectors are useful for reprogramming cell differentiation.

In all aspects, the cell is either a stem cell, like an embryonic, neonatal, fetal, juvenile or adult stem cell, or a primary cell, like fetal, juvenile or adult primary cell.

In one embodiment for the inducible viral vector, the inducible regulation is through an operon.

In a further embodiment, the operon is the Tet operon.

The invention is directed to the following cell lines: pEPEG-BG01V and the pEPOG-BG01V cell line.

The invention is directed to a method for reprogramming cells comprising introducing the plasmid and/or viral vectors of the invention in to the cell; expressing one or more polypeptides encoded thereof in the cell under appropriate culturing conditions; identifying whether the cell has been reprogrammed.

The invention is also directed to double stranded RNA sequences directed to the Oct 4 promoter.

The invention is also directed to a method for reprogramming cells comprising introducing or expressing one or more small RNA molecules into a cell; identifying whether the cell has been reprogrammed, wherein the small RNA molecules interacts with the promoter region of a stem cell marker gene.

In certain aspects, the invention is directed to a method for reprogramming cells comprising introducing the plasmid and/or viral vectors of the invention in to the cell and/or double stranded RNA sequences directed to a stem cell marker or a cell-specific marker.

The invention is further directed to a method of producing a population of reprogrammed stem cells comprising: introducing the vector compositions of the invention into a cell; expressing one or more polypeptides encoded thereof in the stem cell under appropriate culturing conditions; identifying whether the stem cell has been reprogrammed; propagating and maintaining the reprogrammed stem cells in culture.

The invention is also directed to a method for reprogramming cells to a more stem-like dedifferentiated state or to direct a cell towards a particular cell lineage, or to reprogram cells like diseased cells, cancer cells, etc. or to reprogram cells to induced pluripotent cells (iPSCs).

The invention is further directed to viral particles comprising the viral vectors generated in this invention. Specifically, the invention is directed to viral particles comprising the nucleic acids defined in SEQ. ID No.: 3, SEQ. ID No.: 7, SEQ. ID No.: 9, SEQ. ID No.: 10, SEQ. ID No.: 11, SEQ. ID No.: 12, SEQ. ID No.: 49. The invention is also directed to viral particles comprising the nucleic acids defined in SEQ. ID No.: 2 and 8 further comprising reprogrammable genes.

The invention is further directed to kits comprising the viral vectors generated in this invention. Specifically, the invention is directed to kits comprising the nucleic acids defined in SEQ. ID No.: 3, SEQ. ID No.: 7, SEQ. ID No.: 9, SEQ. ID No.: 10, SEQ. ID No.: 11, SEQ. ID No.: 12, SEQ. ID No.: 49. The invention is also directed to kits comprising the nucleic acids defined in SEQ. ID No.: 2 and 8 further comprising reprogrammable genes.

In certain aspects, the invention is directed to methods for producing an induced pluripotent cell (iPSC) by (i) introducing the nucleic acid molecules of the invention (plasmid vectors, viral vectors), either alone or in combination, into a cell; (ii) expressing one or more polypeptides encoded thereof in said cell under appropriate culturing conditions; (iii) identifying whether said cell has been reprogrammed. In another aspect, the invention is directed to induced pluripotent cell (iPSC) produced by the methods defined above.

In a specific aspect, the invention is directed to an isolated nucleic acid molecule comprising (a) an OriP site, (b) a DNA segment encoding the EBNA1 gene under a constitutive promoter; (c) one or more att recombination sites; and (d) a DNA segment encoding at least one selectable marker.

In another specific aspect, the invention is directed to an isolated nucleic acid molecule comprising (a) an OriP site, (b) a DNA segment encoding the EBNA1 gene under an inducible promoter; (c) one or more att recombination sites; and (d) a DNA segment encoding at least one selectable marker.

In another specific aspect, the invention is directed to an isolated nucleic acid molecule comprising: (a) all or part of a baculoviral genome; (b) an OriP site; (c) one or more att recombination sites; (d) a DNA segment encoding the EBNA1 gene under a constitutive promoter; and (e) at least one selectable marker; (f) optionally, a WPRE and/or a VSV-G element.

In another specific aspect, the invention is directed to an isolated nucleic acid molecule comprising: (a) all or part of a baculoviral genome; (b) an OriP site; (c) one or more att recombination sites; (d) a DNA segment encoding the EBNA1 gene under an inducible promoter; and (e) at least one selectable marker; (f) optionally, a WPRE and/or a VSV-G element.

### DESCRIPTION OF DRAWINGS

FIG. 1A: pCEP plasmid vector. Size=10,186 bp.

FIG. 1B: Seq. ID No.: 1. Sequence of the pCEP vector.

FIG. 2A: pBacMam Version 1 DEST construct with CMV promoter. Size=7280 bp.

FIG. 2B: Seq. ID No.: 2. Sequence of the pBacMam Version 1 DEST construct with CMV promoter.

FIG. 3A: pBacMam Version 1 DEST construct without a promoter. Size=6671 bp.

FIG. 3B: Seq. ID No.: 3. Sequence of the pBacMam Version 1 DEST construct without a promoter.

FIG. 4A: pEBNA-DEST plasmid. Size=10,641 bp.

FIG. 4 B: Seq. ID No.: 4. Sequence of the pEBNA-DEST plasmid.

FIG. 5A: pEBNA-DEST plasmid with the EFla promoter driven GFP construct. Size=11,563 bp.

FIG. 5 B: Seq. ID No.: 5. Sequence of the pEBNA-DEST plasmid with the EFla promoter driven GFP construct.

FIG. 6A: pEBNA-DEST plasmid with the Oct4 promoter driven GFP construct. Size=13,588 bp.

FIG. 6 B: Seq. ID No.: 6. Sequence of the pEBNA-DEST plasmid with the Oct4 promoter driven GFP construct.

FIG. 7A: pBacMam Version 1 DEST construct with Tet Operon and EBNA/OriP. Size=15,523 bp.

FIG. 7 B: Seq. ID No.: 7. Sequence of the pBacMam Version 1 DEST construct with Tet Operon and EBNA/OriP.

FIG. 8A: pBacMam Version 2 construct. Size=9762 bp.

FIG. 8 B: Seq. ID No.: 8. Sequence of the pBacMam Version 2 construct.

FIG. 9A: pBacMam Version 2 construct with a CMV promoter driven GFP. Size=8830 bp.

FIG. 9B: Seq. ID No.: 9. Sequence of the pBacMam Version 2 construct with a CMV promoter driven GFP.

FIG. 10A: pBacMam Version 2-DEST construct without any promoter. Size=8851 bp.

FIG. 10 B: Seq. ID No.: 10. Sequence of the pBacMam Version 2-DEST construct without any promoter.

FIG. 11A: pBacMam Version 2-DEST construct without any promoter, with EBNA/OriP. Size=13,708 bp.

FIG. 11B: Seq. ID No.: 11. Sequence of the pBacMam Version 2-DEST construct without any promoter, with EBNA/OriP.

FIG. 12A: pBacMam Version 2-DEST construct with Tet Operon. Size=7883 bp.

FIG. 12 B: Seq. ID No.: 12. Sequence of the pBacMam Version 2-DEST construct with Tet Operon.

FIG. 13: Cloning Schematic for making 4 in 1 and 3 in 1 constructs for generating iPSCs.

FIG. 14: Cloning Strategy for generating BacMam vectors.

FIG. 15: Schematic workflow for inducing Oct 4 gene expression by promoter-targeted double stranded RNA.

FIG. 16A: pBacMam Version 1 DEST construct with EBNA/OriP and the hygromycin selection marker. Size=13,488 bp.

FIG. 16B: Seq. ID No.: 49. Sequence of the pBacMam Version 1 DEST construct with EBNA/OriP and the hygromycin selection marker.

### DETAILED DESCRIPTION

### A. Definitions

In the description that follows, a number of terms used in cell biology (e.g., stem cell biology) and recombinant nucleic acid technology are utilized extensively. Unless defined otherwise, all technical and scientific terms used herein have the same meaning as commonly understood by one of ordinary skill in the art to which this invention is related. One skilled in the art will further recognize many methods and materials similar or equivalent to those described herein, which could be used in the practice of the present invention. Indeed, the present invention is in no way limited to the methods and materials described. For a clear and more consistent understanding of the specification and claims of the present invention, including the scope to be given to such terms, the following terms are defined below.

Stem Cell (SC): As used herein, the term “stem cell” may be an unspecialized, ‘self-renewing’ cell capable of developing into a variety of specialized cells and tissues. Self-renewing may mean that the cells have an ability to divide for indefinite periods (i.e., they do not undergo senescence, or can divide beyond twenty population doublings, which may be typical for a non-renewing cell) in appropriate culture conditions, while giving rise to a specialized cell under specified culture conditions. Self-renewal may be under tight control of specific molecular networks.

“Embryonic stem cells” (ESCs) are undifferentiated cells found in early embryos, and typically are derived from a group of cells called the inner cell mass, a part of the blastocyst. Embryonic stem cells are self-renewing and can form all specialized cell types found in the body (they are pluripotent). ESCs include ECSs of human origin (hESCs) and ESCs of non-human or animal origin. ESCs can typically be propagated, under appropriate conditions, without differentiation, due to their self-renewing properties.

“Embryonic germ cells” are pluripotent stem cells that are typically derived from early germ cells (those that would become sperm and eggs). Embryonic germ cells (EG cells) are thought to have properties similar to embryonic stem cells.

“Multipotent” or “pluripotent” stem cells as used herein, have the ability to develop into more than one cell type of the body. However, pluripotent cells generally cannot form so-called “extra-embryonic” tissues such as the amnion, chorion, and other components of the placenta. Pluripotency may be demonstrated by providing evidence of stable developmental potential even after prolonged culture, and can form derivatives of all three embryonic germ layers from the progeny of a single cell, and by showing the ability to generate a teratoma after injection into an immunosuppressed mouse. Pluripotency may be under tight control by specific molecular networks.

“Totipotent stem cells” have the ability to give rise to all the cell types that make up the body, plus all of the cell types that make up the extraembryonic tissues such as the placenta.

A “progenitor cell” may be an early descendant of a stem cell that can differentiate, and have a capacity to differentiate into a specific type of cell. Progenitor cells are more differentiated than stem cells. Sometimes, the terms “stem cell” and “progenitor cell” may be found to be equated in literature.

“Adult stem cells” may be obtained from, among other sources, blood, bone marrow, brain, pancreas, skin and the fat of adult bodies. Adult stem cells can renew themselves and differentiate to give rise to a limited repertoire of specialized cell types, usually of the tissue type from which it originated. In certain cases, some adult stem cells, under certain growth conditions, can give rise to cell types associated with other tissues (multipotent).

“Somatic stem cells” are non-embryonic stem cells that are not derived from gametes (egg or sperm cells). These somatic stem cells may be of fetal, neonatal, juvenile or adult origin.

Directed differentiation: Manipulating stem cell culture conditions to induce differentiation into a particular cell type. The process whereby an undifferentiated embryonic cell acquires the features of a specialized cell such as a heart, liver, or muscle cell.

“Plasticity”: The ability of stem cells, from one type of differentiated tissue, to generate the differentiated cell types of another tissue.

Desired genes expressed in certain aspects of the invention are “reprogramming or reprogrammable genes.” As used herein, the phrase “reprogramming or reprogrammable genes” may be a gene(s), or target nucleic acid segments of developmental genes, or “stem cell marker genes”, which when expressed in a given cell alter the given cell's phenotype to a different phenotype, due to the expression of one or more reprogrammable gene products. Reprogramming may be done for any reason, for example, to achieve a less differentiated status in certain instances, or a more differentiated status, or for directed differentiation. That is, reprogramming could be done to alter the differentiation capacity of a cell. For instance, methods of the invention may achieve a more stem-like status from a more differentiated stage; or a more non-cancerous state from a cancer state, or disease-free state from a diseased cell, etc. As discussed earlier, “reprogramming or reprogrammable genes” may also refer to “stem cell marker genes” like Oct4 (also termed Oct-3 or Oct3/4), Sox2, c-Myc and Klf4; Oct3/4, Nanog, SSEA1 (Stage Specific Embryonic Antigens), TRA1-80, etc. genes, which are useful for reprogramming cells.

“Developmental genes” or “stem cell markers”: Expression of a given gene, or the activity of its promoter, may be limited to a specific stage of development, cell lineage or cell type, differentiation state. The promoters of such genes may collectively be referred to as developmental promoters. The genes which are normally associated with these promoters are developmentally regulated genes. A number of stem cell specific developmental genes are discussed in this invention. Stem cell markers include, but are not limited to, genes such as Oct4 (also termed Oct-3 or Oct3/4), Sox2, c-Myc and Klf4; Oct3/4, Nanog, SSEA1 (Stage Specific Embryonic Antigens), TRA1-80, etc. Unique expression markers are also used to characterize various stem cell populations such as CD34, CD133, ABCG2, Sca-1, etc. for hematopoietic stem cells; STRO-1, etc. for mesenchymal/stromal stem cells; nestin, PSA-NCAM, p75 neurotrophin R(NTR), etc. for neural stem cells.

Differentiated germ layers also have unique markers for neurons (bIII tubulin, Nestin), mesoderm (SMA, smooth muscle actin), and endoderm (alpha fetal protein).

“Induced pluripotent stem cells” (iPSCs) may be partially or completely differentiated cells that can be reprogrammed to a more embryonic stem cell-like state by being forced to express genes or factors important for maintaining their ‘sternness,’ like ESCs.

An “embryonic stem cell line” may be generated when embryonic stem cells are cultured under in vitro conditions that allow for proliferation without differentiation for months to years; that is, they do not undergo senescence, or can divide beyond twenty population doublings, which may be typical for a non-renewing cell.

A “teratoma” may be established by injecting putative stem cells into mice with a dysfunctional immune system. Since the injected cells cannot be destroyed by the mouse's immune system, these cells survive and form a multi-layered benign tumor called a teratoma. Even though tumors are not usually a desirable outcome, in this test, the teratomas serve to establish the ability of any stem cell to give rise to all cell types in the body. This may be because the teratomas contain cells derived from each of the three embryonic germ layers.

“Primary cells” may be a cell obtained from any given tissue, (e.g., skin giving rise to keratinocyte or melanocyte primary cultures) that can be propagated in vitro under appropriate cell cultures for a limited number of generations, (i.e., they quickly undergo senescence), because primary cells are not modified (or immortalized) for unlimited cell proliferation. Since they are not immortalized, their genomic and/or cell function, data derived thereof are generally considered to be closer to in vivo conditions than data obtained from, say, an immortalized cell line.

As used herein, a “promoter” may be a transcriptional regulatory sequence, or may be a nucleic acid generally located in the 5′-region of a gene, or proximal to either a start codon, or a nucleic acid that encodes for an untranslated RNA. Transcription of an adjacent nucleic acid segment would typically initiated at or near the promoter.

Promoters may be, furthermore, either constitutive or regulatable (e.g., inducible and/or repressible).

“Inducible promoter” may be one where gene expression is controlled by an external stimulus called an “inducer” or “inducing agent”. Inducible elements are DNA sequence elements which act in conjunction with promoters and bind either repressors (e.g. Tet repressor system in E. coli) or inducers (e.g. gall/GAL4 inducer system in yeast). Examples of inducible promoters or expression systems thereof include tetracycline or lactose operons, heat shock proteins (hsp70) operons, metal-inducible promoters, steroid hormone-inducible promoters, etc. Inducible promoters can be said to be regulatable.

A “constitutive promoter” may be a promoter where gene expression under this promoter is generally on, or expressed without any external stimulus and may not be subject to inhibition by a repressor. Generally, for the purposes of this invention, strong promoters like viral promoters are used to achieve high efficiency expression of genes. Efficiency of constitutive promoters can vary and can be influenced, for instance, by metabolic conditions.

A “repressible” promoter's rate of transcription decreases in response to a repressing agent. The “repressors” that inhibit the promoter may be small molecules or proteins. The repressor may be added to the cell or can be co-expressed, for example, through an “operon”. Examples of such an operon useful in the invention include the Tet repressor operon. Here, transcription may be virtually “shut off” until the promoter is derepressed or induced, at which point transcription may be “turned-on.” Repressible promoters can be said to be regulatable.

An “operon” may be a functioning unit of nucleic acid segments, which includes an operator, a common promoter, and one or more structural genes, which are controlled as a unit to produce messenger RNA (mRNA), in the process of transcription.

“Tissue specific promoters” control gene expression in a tissue-dependent manner and according to the developmental stage. Transgenes driven by tissue-specific promoters will only be predominantly expressed in tissues where the transgene product may be desired, mostly leaving the rest of the tissues in an animal/plant unmodified by the transgene expression. Tissue-specific promoters may be induced by endogenous or exogenous factors, so they can be sometimes be classified as inducible promoters or repressible promoters. While it may be preferable to use promoters from homologous or closely related species to achieve efficient and reliable expression of transgenes in particular tissues, promoters from unrelated species with reliable and efficient expression may be used in certain instances.

“Isolated” when used in reference to a nucleic acid molecule or other biological molecule (e.g., a protein) means that the molecule is in high concentration with respect to other molecules of the same type. In other words, a nucleic acid molecule (e.g., a DNA molecule) is said to be “isolated” when the nucleic acid molecule makes up greater than at least 50% (e.g., greater than 50%, greater than 60%, greater than 70%, greater than 80%, greater than 90%, greater than 95%, greater than 97%, or greater than 99%) of the total nucleic acid present, either by total weight or number of molecules present. The same applies for other biological molecules as well.

“Nucleic acid segment” or “DNA segment” (used interchangeably herein as appropriate) may be either all of or a region of a nucleic acid molecule. In many instances, nucleic acid segments may contain, comprise or encode a gene product or a gene, a restriction site, a recombination site, an origin of replication, a regulatory sequence, a promoter sequence, an enhancer sequence, a polyadenylation (poly A) sequence, or any other regulatory or recognition sequence.

A “vector” is a replicable nucleic acid molecule which may be transferred between cells. Examples of vectors include, but may not be limited to, plasmids, bacteriophages (such as phage λ), bacterial artificial chromosomes (BACs), yeast artificial chromosomes (YACs), or viral vectors, such as those based upon lentiviruses, adenoviruses, baculoviruses, etc. Vectors may be designed so that nucleic acid segments may be introduced into them. One aspect of the invention refers to “plasmid vectors” which are replicable nucleic acid molecules that do not comprise viral backbone sequences, or predominantly do not comprise large portions of viral sequences. As will

“Viral vectors”, which form a part of the invention, may be used to efficiently deliver large amounts of genetic material into cells. Delivery of genes by a virus or viral vector may be termed transduction and the infected cells are described as transduced. The reconstruction of viral vectors typically involves the removal of portions of the viral genome, that is, parts that encode for or regulate undesired or dispensable viral functions, for e.g., those involved in viral replication or infection, etc., in a mammalian cell. The minimal “viral genome DNA backbone” may be designed for efficient delivery of large amounts of genetic material. In addition, viral vectors of the invention typically comprise suitable sites to enable cloning of multiple reprogrammable genes, for e.g., any suitable recombinational cloning system like the MultiSite Gateway® cloning system, the EBNA1-OriP system for the episomal maintenance, etc. A typical viral genome (adapted for generation of the vector) may be an insect virus genome, although other viral genomes (for e.g., adenovirus, retrovirus, lentivirus, etc.) can also be adapted. A typical insect virus used here may be a baculovirus, although other non-mammalian viruses are also useful.

Methods of the invention can use viruses of the family Baculoviridae (commonly referred to as baculoviruses) to express exogenous genes in insect cells. In addition to the Baculoviridae family, other viruses which naturally multiply only in invertebrates (for example, MNPV, SNPV virus, and other viruses listed in Table 1 of U.S. Pat. No. 5,731,182, the contents of which are incorporated by reference in their entirety herein) are useful for gene delivery in this invention.

Novel gene delivery viral vectors were developed in this invention that do not stably integrate into the cell's genome, but instead, are either (i) maintained stably episomally due to constitutive expression of the EBNA1 gene, or (ii) that can be induced to sustain reprogramming gene expression during the period of reprogramming due to inducible expression of the EBNA1 gene, and later, can be turned off once cells have been reprogrammed, or the desirable level of reprogramming has been achieved. These gene delivery viral vectors can introduce one or more reprogramming genes at a given time into a given mammalian cell. Viral vector systems generally use an insect virus as a gene delivery system (for example, baculovirus); in this invention BacMam Ver 1 and BacMam Ver 2 family of vectors described in Table 1 were used. The vectors carry one or more genes, or a set of reprogramming genes, into mammalian cells. The backbone of the baculovirus is used to generate BacMam viral vectors. The Ver 2 family of BacMam vectors described in Table 1, namely [SEQ ID NOs: 8, 9, 10, 11, 12] additionally comprise the WPRE (WoodChuck Hepatitis Posttranscriptional Regulatory Element) and the VSV-G expression cassette (Vesicular Stomatitis Virus G protein), which mediates viral entry into a variety of mammalian cells. The viral vectors of the invention are defined in Table 1 (see Examples).

One main purpose for expression vectors is controlled expression of a desired gene inside a host cell or organism. Control of expression may be often desirable to insert the target DNA into a site that is under the control of a particular promoter. In general, an expression vector may have one or more of the following features: a promoter, promoter-enhancer sequences, at least one selection marker, at least one origin of replication, inducible element sequences, repressible element sequences, epitope-tag sequences, and the like.

Recombinational cloning systems (for e.g., Gateway or MultiSite Gateway®), etc.), may be used to generate “expression cassettes” of one of more genes to be expressed in the invention. An “expression cassette” comprises the desired gene to be expressed driven by a promoter (e.g., a native promoter, or any other desired promoter selected to achieve a certain level of expression, or to achieve appropriate temporal expression, or to achieve expression in a desired cell or tissue, etc.) A given vector may contain one or more (e.g., two, three, four, five, seven, ten, twelve, fifteen, twenty, thirty, fifty, etc.) genes, or sets of genes, or one or more portions of genes.

The vectors of the invention may utilize genes that encode for a “selectable marker”. As used herein, the phrase “selectable marker” may be any marker gene that, upon introduction into the host cell, permits the separation of that cell because of the expression of the marker within the cell from cells which do not express the marker. In certain embodiments, the marker gene integrates into the host genome. In other embodiments, the marker does not integrate into the host genome, and instead remains in an episomal vector. A “selectable marker” may be expressed constitutively, inducibly, or its expression may be repressed due to the co-expression of repressor agents or proteins that inhibit their expression.

Suitable selectable markers include, but are not limited to, antibiotic resistance genes like the tetracycline, neomycin, blasticidin, hygromycin, ampicillin, puromycin, etc. and other suitable antibiotics known in the art. Selectable markers may also include, but not be limited to, fluorescent protein genes including but not limited to green fluorescent proteins and its modified mutants, red fluorescent proteins and its modified mutants, etc. Selectable markers may also include but not be limited to genes like the chloramphenicol transferase gene (CAT), hypoxanthine phosphribosyl transferase gene, dihydrooratase gene, glutamine synthetase gene, histidine D gene, carbamyl phosphate synthase gene, dihydrofolate reductase gene, multidrug resistance 1 gene, aspartate transcarbamylase gene, xanthine-guanine phosphoribosyl transferase gene, adenosine deaminase gene, thymidine kinase gene, etc.

“Regulatory sequences” include promoters, enhances, repressors, introns, poly A sequences, 3′ UTRs, etc. known and used by skilled people in the art.

Nucleic acid molecules which may be introduced into host cells include those, but are not limited to, that contain (1) a gene or a set of genes that can reprogram a cell's developmental stage, (2) one or more transcriptional regulatory sequence (such as a promoter, enhancer, repressor, etc.) that can manipulate the expression of a gene or genes placed downstream, (3) an origin of replication (ORI), (4) one or more selectable markers which include antibiotic resistance genes, (5) one or more cloning entry sites, (6) one or more restriction sites, as well as other components. In some embodiments of the invention, the host cell may be a “stem cell.”

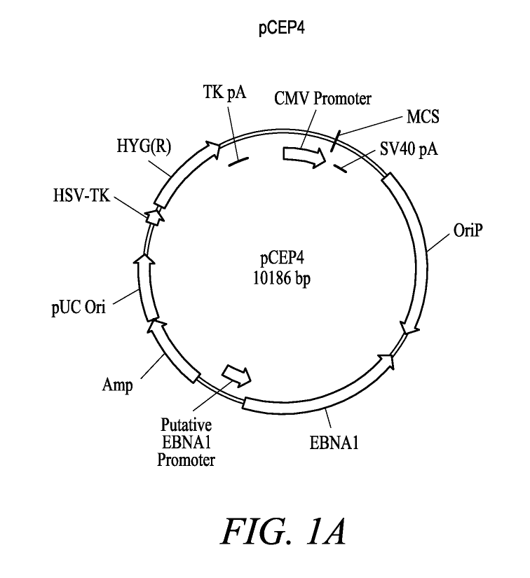

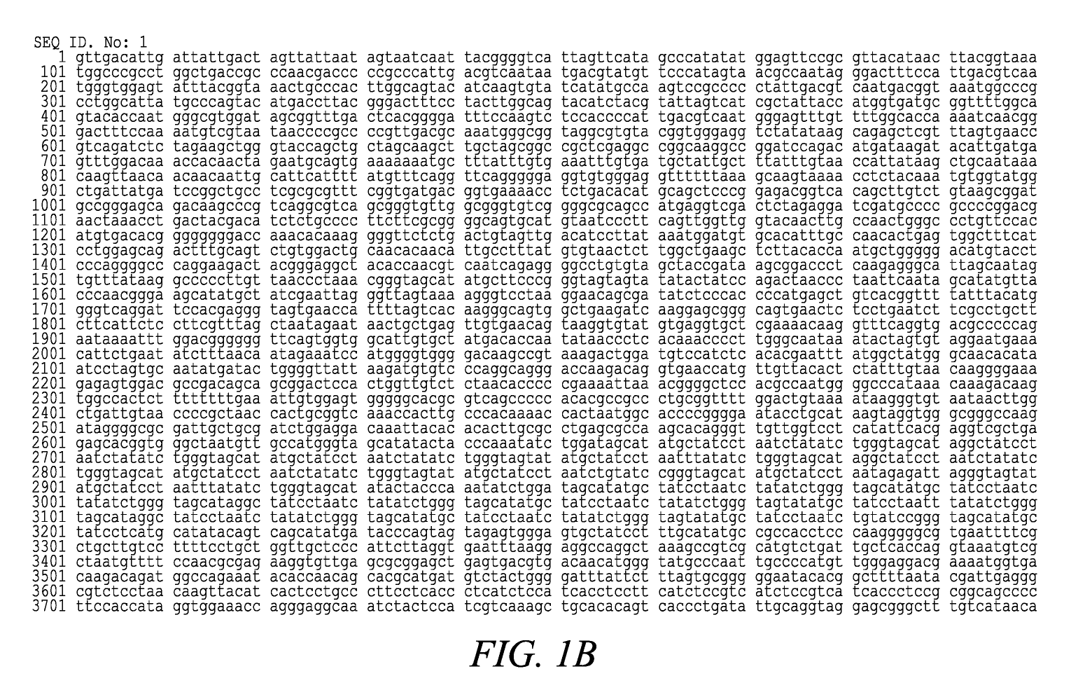

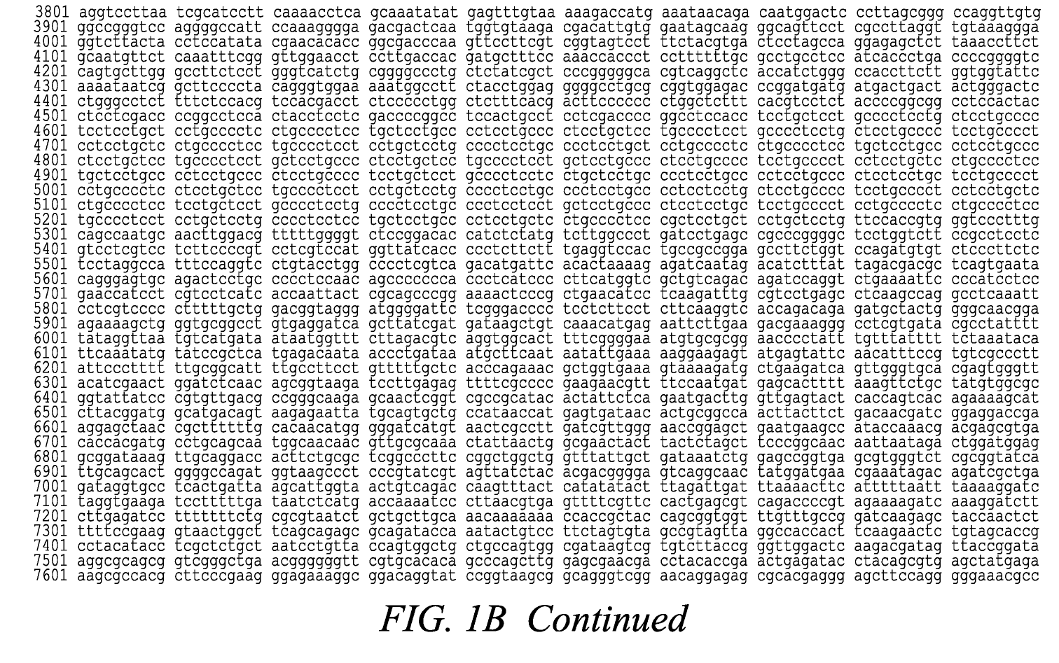

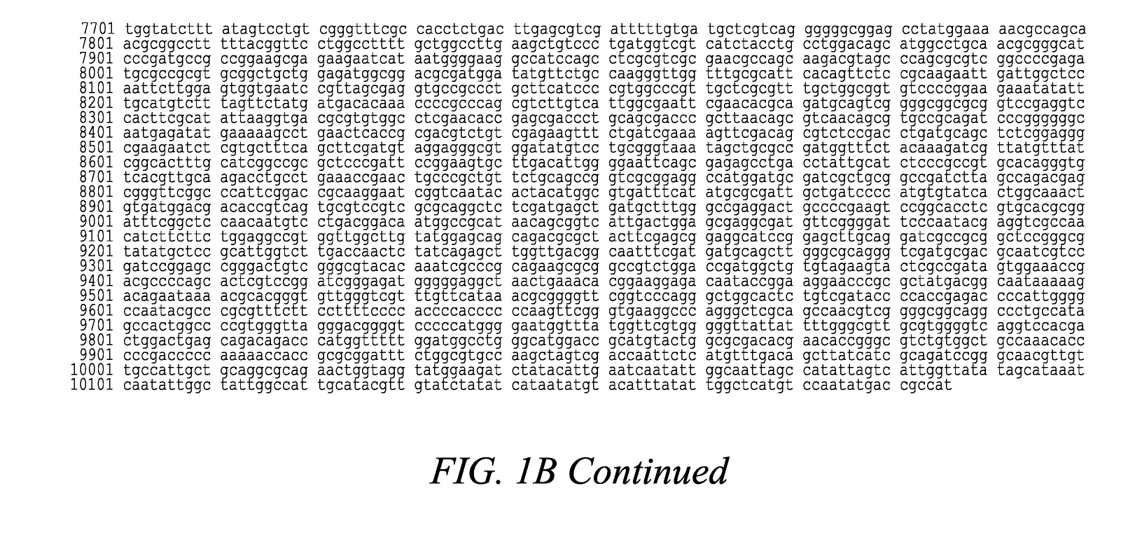

As used herein, the phrase “recombination site” may be a recognition sequence on a nucleic acid molecule which participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences described herein, and mutants, fragments, variants and derivatives thereof, which are recognized by the recombination protein Int and by the auxiliary proteins integration host factor (IHF), FIS and excisionase (Xis). (See Landy, Curr. Opin. Biotech. 3:699-707 (1993).)

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site which flanked by recombination sites.

Examples of recombination sites which may be used in the practice of the invention include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

As used herein, the phrase “recombinational cloning” may be a method, such as that described in U.S. Pat. Nos. 5,888,732 and 6,143,557 (the contents of which are fully incorporated herein by reference), whereby segments of nucleic acid molecules or populations of such molecules are exchanged, inserted, replaced, substituted or modified, in vitro or in vivo.

Recombinational cloning includes methods which involve use of the Gateway® system (Invitrogen Corp., Carlsbad, Calif.).

“MultiSite Gateway®” is a recombinational cloning systems in which more than two nucleic acid molecules are combined to form a single nucliec acid molecule. In one example, a vector may contain four recombination sites designated S1, S2, S3, and S4, none of which will recombine with each other. One nucleic acid segment inserts into the vector by recombination with sites S1 and S2 and another nucleic acid segment inserts into the vector by recombination with sites S3 and S4. Thus, a new recombined vectors is produced which contains both nucleic acid segments. “MultiSite Gateway®” embodiments are described in U.S. Patent Publication No. 2004/0229229 A1, the entire disclosure of which is incorporated herein by reference. As one skilled in the art would understand, recombination systems other than the Gateway® system may be used in the practice of the invention.

As used herein, the term “short RNA” encompasses RNA molecules described in the literature as “tiny RNA” (Storz, Science 296:1260-3, 2002; Illangasekare et al., RNA 5:1482-1489, 1999); prokaryotic “small RNA” (sRNA) (Wassarman et al., Trends Microbiol. 7:37-45, 1999); eukaryotic “noncoding RNA (ncRNA)”; “micro-RNA (microRNA)”; “small non-mRNA (smRNA)”; “functional RNA (fRNA)”; “catalytic RNA” [e.g., ribozymes, including self-acylating ribozymes (Illangaskare et al., RNA 5:1482-1489, 1999]; “small nucleolar RNAs (snoRNAs)”; “tmRNA” (a.k.a. “10S RNA”, Muto et al., Trends Biochem. Sci. 23:25-29, 1998; and Gillet et al., Mol. Microbiol. 42:879-885, 2001); RNAi molecules including without limitation “small interfering RNA (siRNA)”, double stranded RNA (dsRNA), “endoribonuclease-prepared siRNA (e-siRNA)”, “short hairpin RNA (shRNA)”, and “small temporally regulated RNA (stRNA)”; “diced siRNA (d-siRNA)”, and aptamers, oligonucleotides and other synthetic nucleic acids that comprise at least one uracil base, and maybe used interchangeably. dsRNA used in the invention may be used to silence or suppress the expression of genes (transcriptional gene silencing: TGS), or to activate the expression of genes (transcriptional gene activation: TGA).

Other terms used in the fields of recombinant nucleic acid technology, molecular and cell biology, particularly stem cell biology, as used herein will be generally understood by one of ordinary skill in the applicable arts.

### B. Detailed Description

The present invention relates, in part, to nucleic acid molecules (e.g., vectors such as plasmids, viral vectors, small RNA molecules), as well as compositions that contain such nucleic acid molecule, that may be used for manipulating or reprogramming cell development. The present invention also relates, in part, to nucleic acid molecules (e.g., vectors such as plasmids, viral vectors, small RNA molecules), that are expressed in a regulatable manner (e.g., either in a constitutive or inducible manner). One example of an application for nucleic acid molecules of the invention is in the conversion of any differentiated stem cell (e.g., adult stem cell) to a more pluripotent ES-like state. The present invention also provides, in part, methods for reprogramming cells (e.g., stem cells), or altering the differentiation capacity of a cell to a more plastic (e.g., less differentiated) state, by either activating, silencing or restoring to normal levels, expression of reprogrammable genes in a regulatable manner (e.g., either in a constitutive or inducible manner).

Reprogramming of any cell, including stem cells, somatic cells, cancer cells, diseased cells, or normal cells, may be achieved using the molecules, compositions and methods described herein. Reprogramming may be done for any reason, for example, to achieve a less differentiated status in certain instances, or a more differentiated status, or for directed differentiation. That is, reprogramming could be done to alter the differentiation capacity of a cell. For instance, methods of the invention may achieve a more stem-like status from a more differentiated stage; or a more non-cancerous state from a cancer state, or disease-free state from a diseased cell, etc. Methods and compositions of the invention used in cell reprogramming may be applicable in a variety of fields which include cancer treatment, tissue remodeling, aging, tissue repair, etc. Whether a particular cell has been reprogrammed may be determined by identifying the expression of specific cell-markers associated with that state, for instance, embryonic or fetal cell markers, reduction in expression of a cancer marker, stem cell marker genes, etc.

Methods of the invention are directed, in part, to gene delivery systems. In many instances, these methods do not result in the stable integration of nucleic acid segments into the cell's genome (e.g., are episomal), and/or result in the expression of reprogramming genes from a vector. Since gene delivery systems of the invention such as this do not integrate into the cell's genome, gene expression may be only sustained while the episomal vector (e.g., an ectopic vector) is maintained within the cells. In certain embodiments, episomal vectors of the invention will segregate along with the chromosome, provided an episome maintaining protein, (e.g., EBNA1) is expressed. In some instances, the episome maintaining protein, (e.g., EBNA1), may be expressed constitutively, where its expression would be driven by, either, its own native promoter, any constitutive promoter known in the art (e.g., CMV promoter), or a cell-type-specific (e.g., liver specific), stage-specific (e.g., ESC), or tissue-specific promoter, etc. In other instances, the episome maintaining protein, (e.g., EBNA1), may be expressed inducibily, where its expression would be driven by any inducible promoter known in the art (e.g., the Tet operon, etc.). Here, the episomal vector would only be maintained as long as the inducer is present. In a broad sense, episomal vectors may be eliminated from cells by methods which involve removal of an inducer.

Methods of the invention are also directed, in part, to small RNA molecule (e.g., dsRNA, RNAi) systems for reprogramming cell (for e.g., stem cell) differentiation.

In certain aspects, the invention relates to compositions and methods for maintaining episomal vectors in cells. Such maintenance may occur in the absence of direct selective pressure (e.g., by the presence of an antibiotic resistance gene and an antibiotic). For example, the episomal vector may contain a nucleic acid segment which allows for the vector to segregate with cellular nucleic acid materials (e.g., cellular chromosomes). An example of such a nucleic acid segment is the Epstein-Ban Virus origin, OriP. In many instances, maintenance of the vector will be dependent upon the presence of the EBNA1 protein which interacts with the OriP nucleic acid segment located in the episomal vector. In other instances, the EBNA1 protein maintains any OriP containing system, which include OriP containing vectors, genomes, nucleic acid segments, etc.

The EBNA1 protein which interacts with the nucleic acid segment located in the episomal vector may be expressed by the same vector, or from a different nucleic acid molecule (e.g., another vector, the cell's chromosome, etc.). Further, the protein may be expressed in a constitutive or regulatable (e.g., inducibly or repressible) manner. Elimination of the protein from the cell may be used to remove the episomal vector from the cell (e.g., by “curing”). As an example, if the protein is expressed on a vector separate from the episomal vector, then the protein may be eliminated from the cell by removal of that expression vector from the cell. As an example, the episomal vector may contain an OriP site and a second vector may contain both an EBNA1 coding region operably linked to a constitutive promoter and an antibiotic resistance marker. In most instances, when selective pressure is removed from the culture medium by omitting the antibiotic to which the marker confers resistance, the vector which encodes the EBNA1 protein will eventually be lost from the culture cells. When this vector is lost from the cultured cells, the EBNA1 protein will no longer be expressed, resulting in the loss of episomal vectors containing OriP sites. Thus, in many instances, no footprint of the vector system is left behind once the inducing agent is removed. This may be desirable when cells need to be reprogrammed only for a short time to achieve a desired differentiation or dedifferentiation level as needed, and after which, remnants of the vectors systems that modify the cell's differentiation status are not desirable. This method would be highly desirable in a clinical medicine setting where patient-specific pluripotent cells, for instance, may be required for disease research, or for cell replacement therapies.

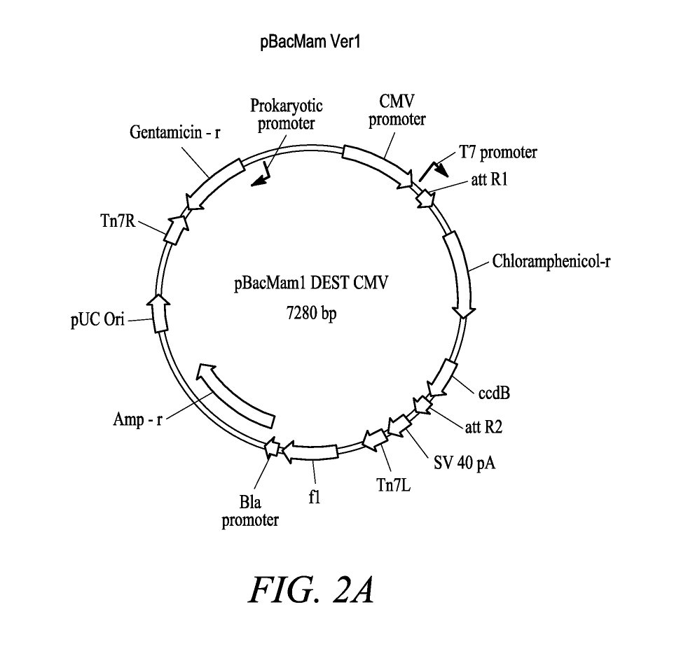

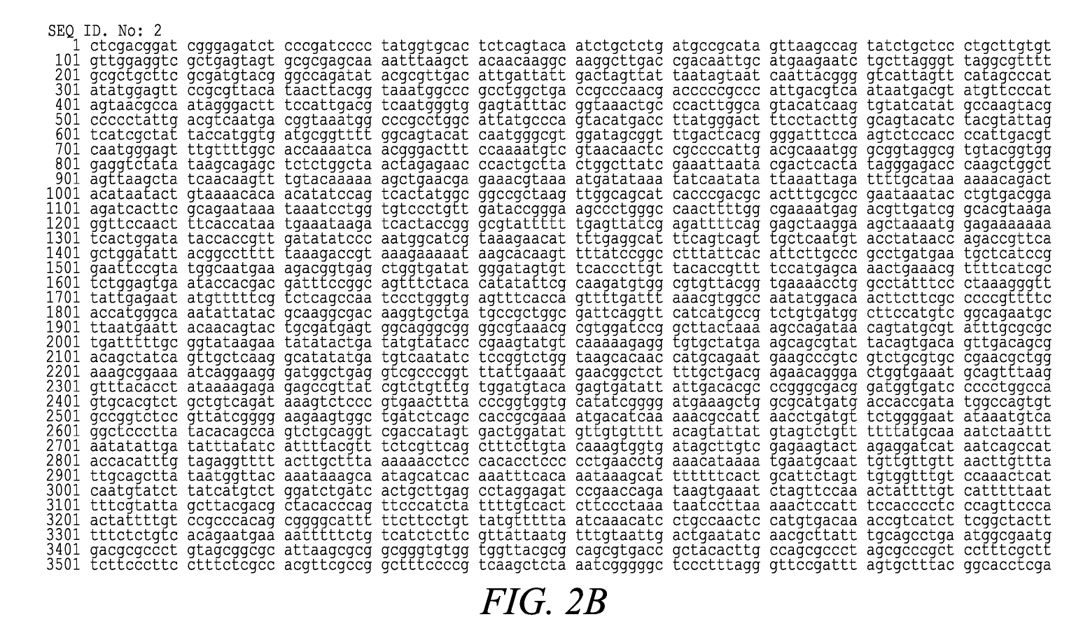

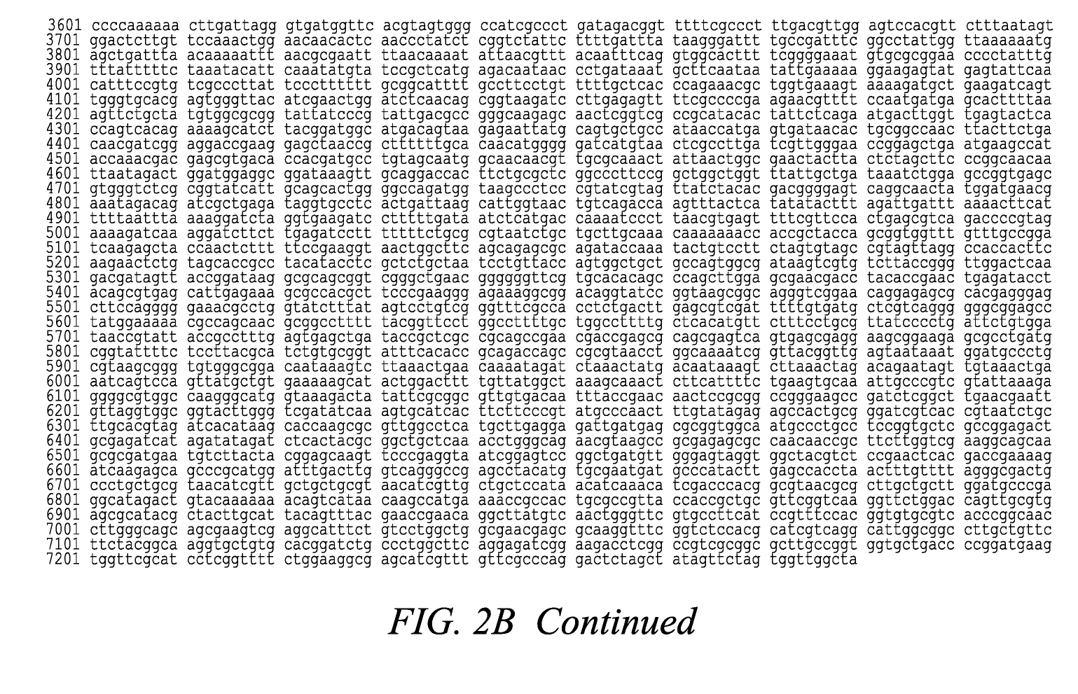

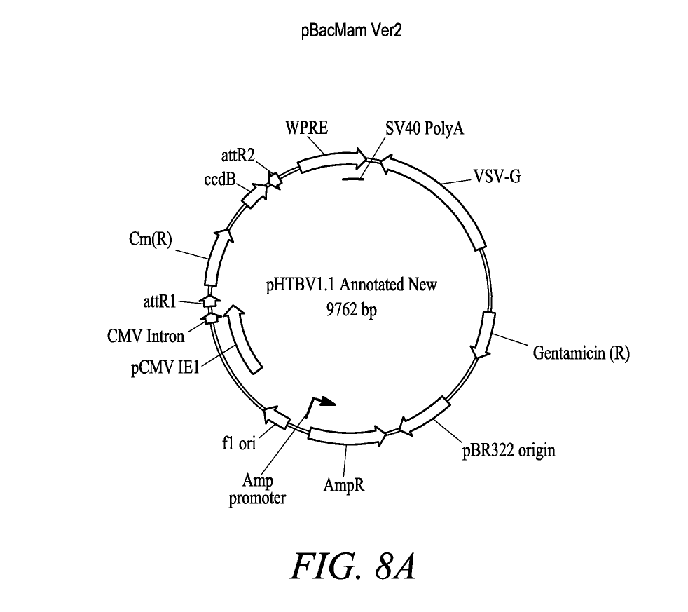

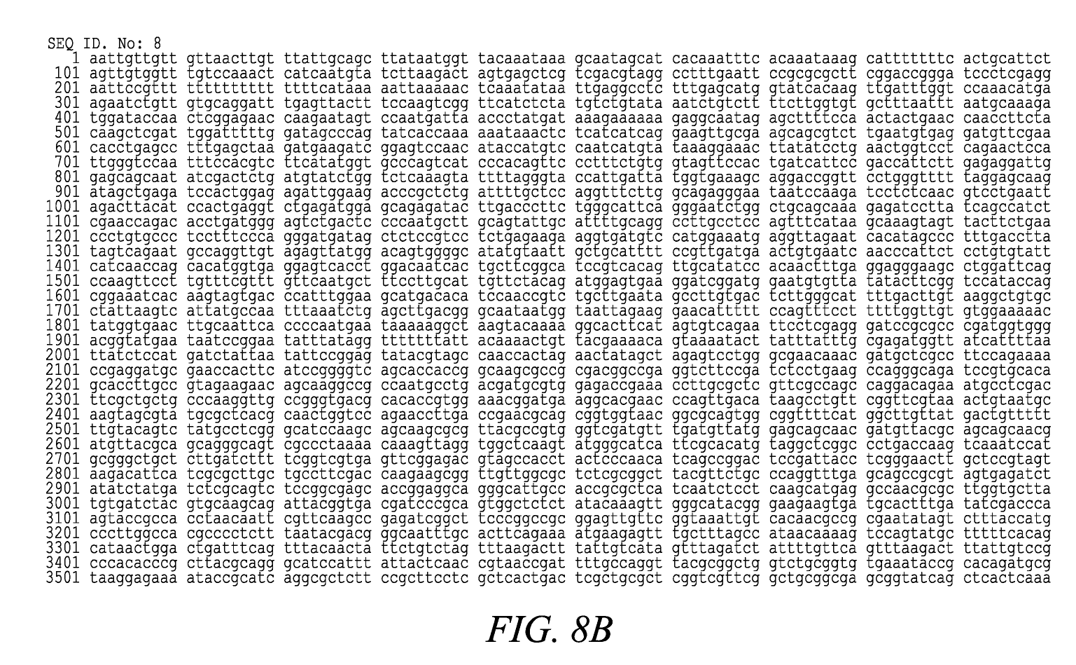

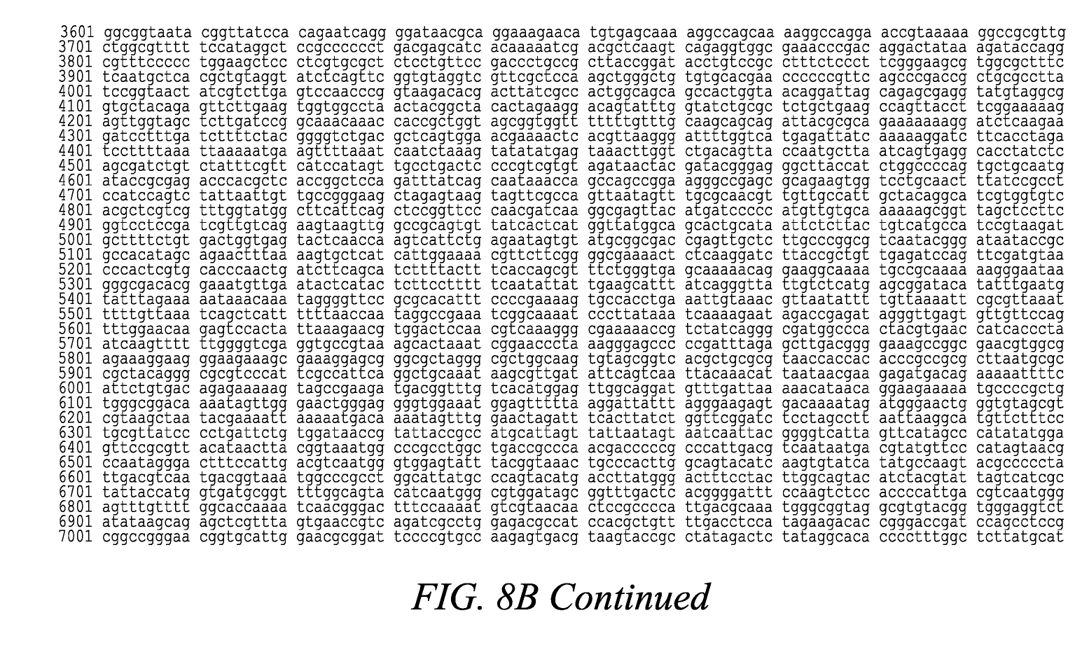

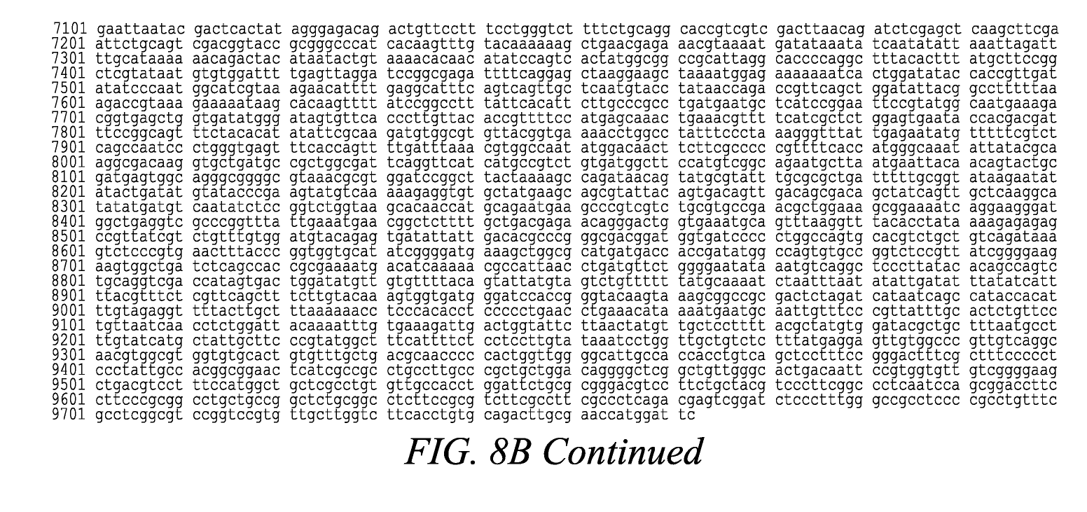

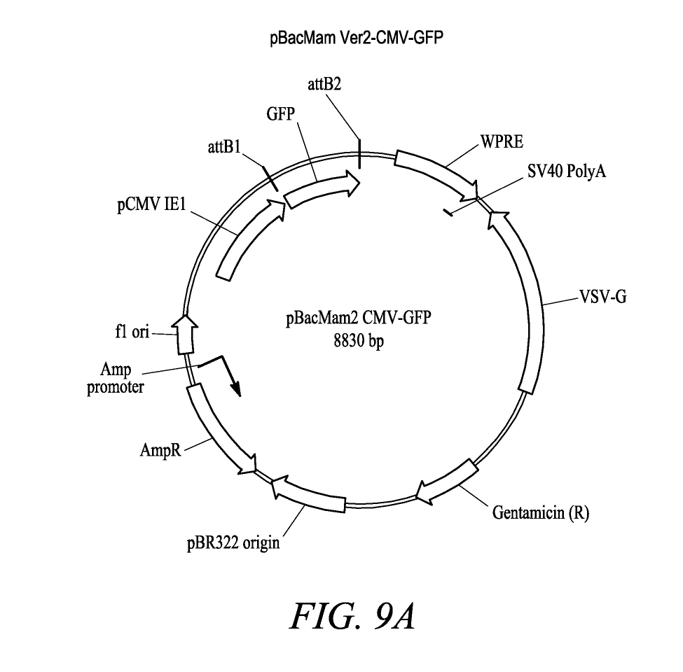

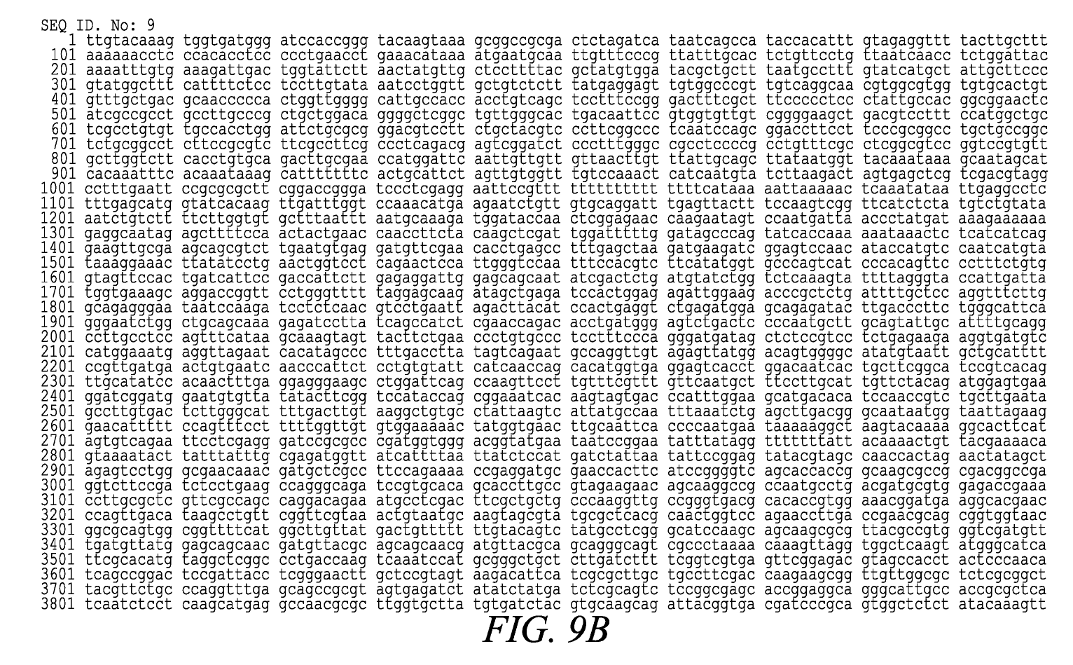

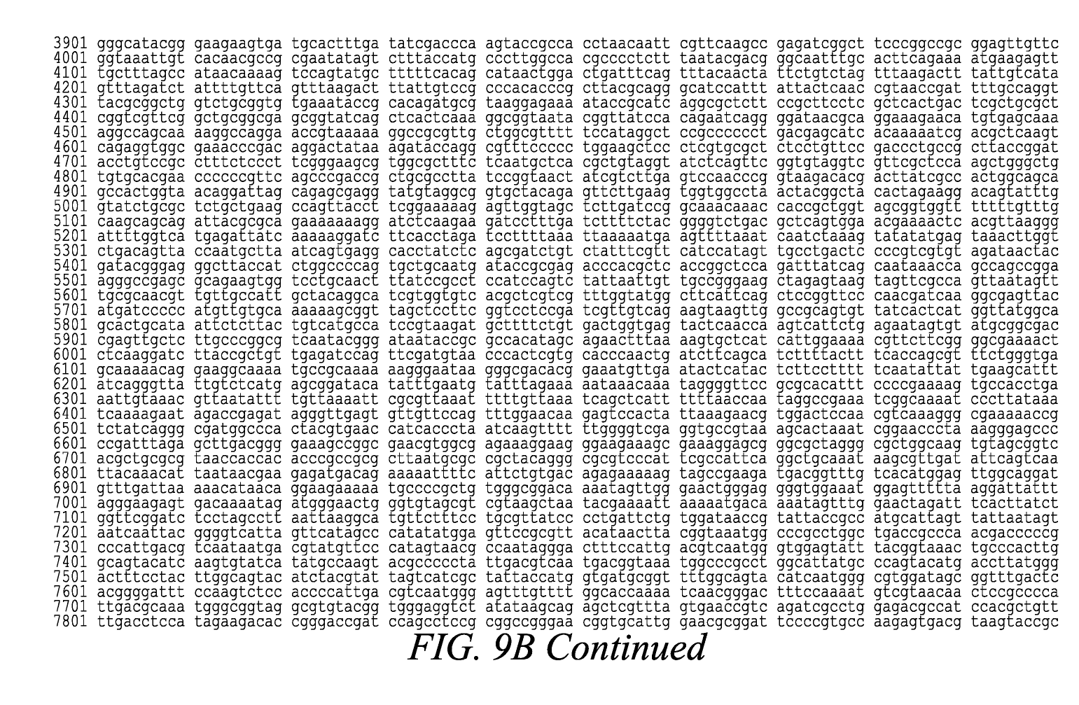

The methods of the invention also use viral vectors without the EBNA/On P system, like the pBacMam Ver 1 {FIG. 2; SEQ ID NO: 2} or the pBacMam Ver 2 that comprises the WPRE and VSV-G elements {FIG. 8; SEQ ID NO: 8}, to reprogram cells. Here, expression of the reprogramming genes expressed by the viral vector occurs only for a short while and requires reprogramming particles to be transduced at intervals of 72 hours, with 2× and 4× treatments, resulting in the formation of colonies with stem cell-like characteristics.

Host Cells

Host cells used in the invention include prokaryotic and eukaryotic cells. In certain aspects, host cells such as bacterial cells, like Eschericia coli, may be used to propagate recombinational molecules like vectors, etc. used in the invention. In other aspects of the invention, the host cell may be an insect cell that may be used to generate and propagate a vector, e.g., an insect vector that may be used in the invention, or for example, to generate viral particles as part of a viral delivery system. In most aspects, host cells which may be employed in the practice of the invention are cells, like stem cells, that may be reprogrammed using reprogrammable genes, e.g., stem cell marker genes. In many instances, host cells may be reprogrammed into a pluripotent embryonic stem cell-like state. Further, the stem cells may be “multipotent” stems cells, or “pluripotent” stem cells.

Typically, host cells used in the invention are mammalian host cells. Mammalian host cells, such as mouse, rat, dog, cat, pig, rabbit, human, non-human primates, etc., non-human animals, in particular from a non-human mammal, may also be used. Host cells may be those of a domestic animal or an agriculturally important animal. An animal may, for example, be a sheep, pig, cow, horse, bull, or poultry bird or other commercially-farmed animal. An animal may be a dog, cat, or bird and in particular from a domesticated animal. An animal may be a non-human primate such as a monkey. For example, a primate may be a chimpanzee, gorilla, or orangutan. Host cells may be rodent cells. However, in some aspects, avian cells, annelid cells, amphibian cells, reptilian cells, fish cells, plant cells, or fungal (particularly yeast) cells may be used as hosts.

An embryonic or adult stem cell may be an unspecialized cell capable of developing into a variety of specialized cells and tissues. Embryonic stem cells may be found in very early embryos or may be derived from a group of cells called the inner cell mass, a part of a blastocyst. Embryonic stem cells may be self-renewing and may form all cell types found in the body (pluripotent). Adult stem cells may be obtained from, among other sources, blood, bone marrow, brain, pancreas, amniotic fluid and fat of adult bodies. Adult stem cells may renew themselves and may give rise to all the specialized cell types of the tissue from which it originated, or in certain instances, potentially, cell types associated with other tissues (multipotent).

Adult cells may be reprogrammed to an embryonic stem cell-like state by the expression of factors important for maintaining the “stemness” of embryonic stem cells (ESCs). For instance, mouse iPSCs may demonstrate important characteristics of pluripotent stem cells, including expression of stem cell markers, forming tumors containing cells from all three germ layers, and/or being able to contribute to many different tissues when injected into a mouse embryos at a very early stage in development. Human iPSCs may further express stem cell markers and/or may be capable of generating cells characteristic of all three germ layers.

Stem cells may be derived from any stage or sub-stage of development, in particular they may be derived from the inner cell mass of a blastocyst (e.g. embryonic stem cells). Host cell types include embryonic stem (ES) cells, which are typically obtained from pre-implantation embryos cultured in vitro. (see, e.g., Evans, M. J., et al., 1981, Nature 292:154 156; Bradley, M. O., et al., 1984, Nature 309:255 258; Gossler et al., 1986, Proc. Natl. Acad. Sci. USA 83:9065 9069; and Robertson, et al., 1986, Nature 322:445 448). ES cells may be cultured and prepared for introduction of the targeting construct using methods well known to the skilled artisan. (see, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357 371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorin et al., 1992, Transgenic Res. 1:101; and Veis et al., 1993, Cell 75:229).

In some cases, cells (e.g., stem cells) may be obtained from, or derived from, extra-embryonic tissues. By way of example, cells (e.g., stem cells) may also be of varied tissue origins including, but not limited to, myeloid, lymphoid, hematopoietic, pancreatic, cardiac, neural, skin, bone, or other tissues. These tissues may be obtained from fetal, neonatal, juvenile or adults.

ES cells may be derived from an embryo or blastocyst of the same species as the developing embryo into which they are to be introduced. ES cells are typically selected for their ability to integrate into the inner cell mass and contribute to the germ line of an individual when introduced into the mammal embryo at the blastocyst stage of development. Thus, any ES cell line having this capability is suitable for use in the practice of the present invention.

It may be possible to produce a transgenic animal from embryonic stem cells in which all of the animals' stem cells contain the engineered gene or genes provided the regulatable (e.g., inducible) selection pressure is maintained for maintenance of the episomal vector. The ability to create such genetically engineered animals allows for the study of effects of reprogramming genes on animal development, protein-protein interactions, and the activity of specific cell signaling pathways in cell development. Whole animal models that may be generated with this platform technology may enable therapeutic studies, drug toxicity testing, and cell (e.g., stem cell) transplant tracking using fluorescent proteins and MRI contrasting reporters. In some embodiments, the use of the invention allows for the creation of adult stem and progenitor ready-engineered populations for genomic manipulation at very early passage numbers. Such ready-engineering stem cells may permit genetic manipulation in non immortal adult stem cells which has been impossible so far. In cases where adult stem cells are used, expression vectors may contain genes that correct genetic errors so that modified stem cells may be returned to the animal as a form of treatment for a particular medical condition.

Any of the cells (e.g., stem cells) described above can be reprogrammed or manipulated using the compositions and methods described herein.

Vectors compositions described herein may be designed to introduce one or multiple reprogrammable genes such as developmental genes or stem cell markers efficiently into cells (e.g., stem cells) by non-integration methods. A variety of viral and non-viral methods, and genome integration methods for introduction of desired nucleic acids (e.g., DNA) into cells (e.g., stem cells) exist. However, these methods involve multi-steps, are laborious, have low efficiency, and in the case of genome integration methods, require characterization and may cause gene disruption and other uncertainties. Episomal vectors offer an appealing alternative since they are relatively free from chromosomal effects associated with genomic integration methods.

In some aspects, the invention is directed to gene delivery vectors comprising components derived from any virus that maintains its genome episomally (for e.g., Epstein-Barr virus (EBV), SV40 virus, adeno-associated virus (AAV), HPV16 virus, etc). Although in most instances, the invention refers to the EBNA1 protein of the EBV virus, also encompassed in the invention is any other equivalent episome maintaining protein or proteins derived from other episomal viruses such as adeno-associated virus (AAV), SV40, BSOLV, HIV-1, etc., and the genes encoding these episomal proteins and/or their OriP elements may also be used to generate vectors of the invention.

In one aspect of the invention, gene delivery vectors, which are either plasmid or viral vectors, may be prepared from components derived from the Epstein-Ban virus, which contains the EBNA-1 gene that encodes the nuclear antigen, EBNA1, and the Epstein-Barr virus origin of replication, OriP.

In one aspect, the invention describes episomal plasmid vectors. The pCEP4 (Invitrogen) vector contains, both, the EBNA1 gene and the origin of replication, OriP. Compositions and methods of the invention are directed to the generation of the pEBNA-DEST vector by removing portions of the pCEP4 vector and replacing it with a ccdB/Cm cassette flanked by attR1 and attR2 recombination sites (see FIG. 1 of Attachment A). In an aspect of the invention, further vectors can be generated by replacing portions of any plasmid vector that harbors episomal viral genome components similar to EBNA1 and OriP, for e.g., the pCEP4 vector, with any other cassette that is flanked by any known recombination cloning sites which have been discussed herein. For instance, one skilled in the art would understand that any recombinational cloning systems (for e.g., Gateway or MultiSite Gateway®, etc.) may be used in the practice of the invention. Typically, a vector may be adapted to the MultiSite Gateway® technology to allow for ease and custom creation of expression cassettes, which may include multi-fragments into one expression construct. MultiSite Gateway® further allows for the choice of any promoter-gene pairing, transcription/translation element pairing, or any regulatable element pairing. The invention thus relates to methods of using episomal EBNA-recombinational gene delivery vectors, as described herein, for reprogramming cells (for e.g., stem cells).

In one aspect, the viral vectors of the invention are used to efficiently deliver large amounts of genetic material into cells (e.g., stem cells). Delivery of genes by a virus is termed transduction and the infected cells are described as transduced. The construction of viral vectors commonly used in gene expression may be based on the principle of removing unwanted functions from a virus that are involved in infection, and/or replication in a mammalian cell. Viral vectors of the invention typically comprise, amongst other elements, the minimal viral DNA backbone for efficient viral delivery and generation of viral particles, recombination based cloning elements (e.g., MultiSite Gateway® cloning cassettes), one or more components of the EBNA1-OriP system (e.g., an OriP segment and, optionally, nucleic acid which encodes the EBNA1 protein) for the episomal maintenance of the vector during mammalian cell division, etc. Recombination based cloning elements enable the cloning of one or multiple reprogrammable genes into the cell. A typical viral vector used in the invention is a baculoviral vector.

Viral vectors may be prepared using one or more of the following: (a) components derived from the Epstein-Barr virus containing the EBNA-1 expression cassette and the OriP origin of replication, (b) a viral DNA backbone, like a baculovirus DNA backbone, to allow for delivery of large amounts of genetic material into cells (e.g., stem cells) using a viral delivery system (e.g., BacMam).

In one embodiment, components may be delivered as two or more modified episomal viral vectors, one vector carrying the EBNA1-OriP and other necessary components, while the second vector carries the MultiSite Gateway® expression cassette(s) and OriP for episomal maintenance. In a second embodiment, the components may be delivered as one modified episomal viral vectors, where one vector carries the EBNAI-OriP, recombinational cloning (e.g., MultiSite Gateway®) expression cassette(s) and other necessary components. In the event that it is desirable to express additional genes, these genes may be introduced in additional recombinational cloning (e.g., MultiSite Gateway®) expression cassette(s) with an OriP site. The invention also provides methods for reprogramming stem cells using the episomal EBNA-viral vectors thus generated and described.

In one aspect, the invention describes constitutive viral (e.g., baculoviral) gene delivery vectors. In another aspect, the invention describes inducible viral (e.g., baculoviral) gene delivery vectors. In constitutive viral vectors, e.g., pEP-FB-DEST1 (Attachment Q), regulation of the episomal protein, e.g., EBNA1, may be under either the native EBNA1 promoter, any constitutive promoter known in the art, or a lineage-specific or tissue-specific promoter. A constitutive promoter may be a strong viral promoter like the CMV promoter. In inducible viral vectors, e.g., pFBbg1-DEST1, (Attachment N), regulation of the episomal protein, e.g., EBNA1, may be under an inducible operon, (e.g., the Tet operon like the CMV/Tet Operon promoter) which drives the expression of the EBNA1 gene. DNA segments expressing each of these elements are found on the vector (Example 2).

Non-mammalian viruses are especially useful for expressing and delivery exogenous genes into mammalian cells. Methods of the invention can use any type of virus to generate viral particles. In many instances, “insect” DNA viruses are used to deliver the genetic material into cells (e.g., stem cells). By “insect” DNA virus is meant a virus, whose DNA genome is naturally capable of replicating in an insect cell (e.g., Baculoviridae, Iridoviridae, Poxyiridae, Polydnaviridae, Densoviridae, Caulimoviridae, and Phycodnaviridae).

In particular, viruses of the family Baculoviridae (commonly referred to as baculoviruses) are useful in this invention. In addition to the Baculoviridae family, other families of viruses which naturally multiply only in invertebrates (for example, MNPV, SNPV virus, and other viruses listed in Table 1 of U.S. Pat. No. 5,731,182, the contents of which are incorporated by reference in their entirety herein) are useful for gene delivery in this invention.

Baculovirus comprising the viral vectors embodied in the invention (e.g. constitutive or inducible BacMam EBNA vectors) may be used to package and deliver desired large DNA constructs to cells, (e.g. ESC, germ cells) to achieve entire gene knockouts and/or delivery of genes, as for instance, in gene therapy. The vectors of the invention may be useful for many purposes, for generating transgenic knockout or overexpressing animals, in gene therapy, for protein production of large proteins, etc. The overall size of these large constructs may be about 15-20 kb, although slightly higher or lower sizes (e.g., 5-10 kb, 10-15 kb, etc.) can also be used, making the overall engineered baculoviral genome to be about 170-180 kb, although slightly higher or lower sizes (e.g., 100-120 kb, 120-140 kb, 140-160 kb, 160-180 kb, 180-200 kb, 200-220 kb, 220-240 kb, 240-260 kb, 260-280 kb, 280-300 kb, etc.) may also be achieved. In some instances, these constructs may contain one or more of the following: 5′ and 3′ homology arms, positive selectable markers, a cassette to express a rare sequence homing endonuclease, (e.g. Isce-I (from a class II mammalian promoter)), etc, to linearize the construct once it is inserted into the cell. Methods and compositions of the invention may be used to package and deliver to cells large constructs, may be entire BACs which could be significantly larger up to 150 kb, to achieve engineered baculoviral genomes of about 300 kb.

Small RNAs

In one aspect, the invention provides compositions and methods for the delivery of small noncoding RNAs, which include micro RNAs siRNAs, dsRNA (double stranded RNA), interfering RNA (RNAi), etc. into cells. Small noncoding RNAs may regulate gene expression at multiple levels like modifying chromatin architecture, transcription, RNA editing, RNA stability, translation, etc. While small RNA or interfering RNA (RNAi) is generally associated with silencing of homologous gene sequences (also termed Transcriptional Gene Silencing: TGS), some small RNAs, like double stranded RNA (dsRNA), may also induce long-lasting sequence specific induction of certain genes (Transcriptional gene activation: TGA).

Interfering RNA molecules may be expressed as “hairpin turn” molecules (e.g., shRNAs), or as two separate RNA strands which are capable of hybridizing to each other (dsRNA). Most molecules which function in RNA interference may contain regions of sequence complementarity of between 18 and 30 nucleotides.

Nucleic acid molecules of the invention may be engineered, for example, to produce dsRNA molecules which when transcribed, folds back upon itself to generate a hairpin molecule containing a double-stranded portion. In certain instances, the double stranded hairpin molecule may be activating or may be inhibitory, depending on its design and the gene it regulates.

In one aspect, dsRNA may be associated with TGA (activation). TGA using dsRNA involves activating expression of those genes associated with differentiation (e.g., developmental genes or stem cell markers such as Oct4, Sox2, c-Myc and Klf4; Oct3/4, Nanog, SSEA1, TRA1-80, etc), or their promoters and/or enhancers sequences, which may result in the reprogramming of the cell away from its original differentiation pathway.

In some instances, dsRNA molecules may be introduced into the cell via transfection (e.g., transient or stable), or via peptide delivery systems (e.g. MPG), or any other suitable delivery system for small RNAs known and used in the art. In other instances, dsRNA molecules may be introduced via any expression cassette in a vector, including the vectors described and provided in this invention. Vectors could be viral, plasmid, bacterial or any other vector that is useful for practicing the invention.

One strand of the double-stranded portion may correspond to all or a portion of the sense strand of the mRNA transcribed from the gene to be silenced, while the other strand of the double-stranded portion may correspond to all or a portion of the antisense strand. Other methods of producing a double-stranded RNA molecule may be used, for example, nucleic acid molecules may be engineered to have a first sequence that, when transcribed, corresponds to all or a portion of the sense strand of the mRNA transcribed from the gene to be silenced and a second sequence that, when transcribed, corresponds to all or portion of an antisense strand (i.e., the reverse complement) of the mRNA transcribed from the gene to be silenced. This may be accomplished by putting the first and the second sequence on the same strand of the viral vector each under the control of its own promoter. Alternatively, two promoters may be positioned on opposite strands of the vector such that expression from each promoter results in transcription of one strand of the double-stranded RNA. In some embodiments, it may be desirable to have the first sequence on one viral vector or nucleic acid molecule and the second sequence on a second vector or nucleic acid molecule and to introduce both molecules into a cell containing the gene to be silenced. In other embodiments, a nucleic acid molecule containing only the antisense strand may be introduced and the mRNA transcribed from the gene to be silenced may serve as the other strand of the double-stranded RNA.

In an example of this embodiment, synthetic RNAi molecules may be designed to silence the expression of genes associated with differentiation, like developmental genes or stem cell markers genes. In other embodiments, a silencing RNA like Stealth™ RNAi may be designed and introduced into EBNA producing cells to suppress the expression of the EBNA1 proteins.

The dsRNA may have one or more regions of homology to the gene. The homology maybe to all or portions of the promoter that drives gene expression of the activating or silencing gene, or, the homology may be to all or portions of the gene itself. Regions of homology may typically be from about 20 bp to about 100 bp in length, from about 20 bp to about 80 bp in length, from about 20 bp to about 60 bp in length, from about 20 bp to about 40 bp in length, from about 20 bp to about 30 bp in length, or from about 20 bp to about 26 bp in length. Typical dsRNA lengths that may be used in the invention are 20 to about 32 bp.

A hairpin containing molecule having a double-stranded region may also be used as RNAi. The length of the double stranded region may be from about 20 bp to about 100 bp in length, from about 20 bp to about 80 bp in length, from about 20 bp to about 60 bp in length, from about 20 bp to about 40 bp in length, from about 20 bp to about 30 bp in length, or from about 20 bp to about 26 bp in length. The non-base-paired portion of the hairpin (i.e., loop) can be of any length that permits the two regions of homology that make up the double-stranded portion of the hairpin to fold back upon one another.

Synthetic RNAi and/or synthetic dsRNA molecules designed and used in the invention may also be used for TGS (silencing genes) of developmental or stem cell genes, their promoters and/or enhancers sequences.

Synthetic RNAi and/or synthetic dsRNA molecules may be designed using the methods described in the invention, and/or, by methods known and practiced in the art. These may include modifications to methods known and practiced in the art.

Another means for cell reprogramming can be by using small molecules that are involved in chromatin modifications. These small molecules include proteins, peptides, small RNA molecules, small chemical molecules, etc. that affect the DNA methylation status of a gene, or the promoter and/or enhancer region of that gene. Methods of reprogramming would include exposing a cell to the small molecule that affects a specific gene of interest. The small molecule may be added to the culture media at an appropriate time, or may be transfected (stably or transiently) into the cell, or may be introduced in an expression vector into the cell and effects reprogramming upon expression of the small molecule.

Molecules that affect chromatin modifications include, broadly, histone deacetylase (HDAC) inhibitors, DNA methyltransferase inhibitors, epigenetic modifiers, molecules affecting cell signaling pathways (for e.g., involved in DNA methylation signaling), etc. Some exemplary small molecules that may be used in the invention include, but are not limited to, 5′-azaC, dexamethasone, valproic acid (VPA), suberoylanilide hydroanic acid (SAHA), sodium butyrate, RG108, BIX01294, PD0325901, CHIR99021, SB431542, BIO, purmorphamine, etc.

In certain aspects, cell culture conditions for reprogramming genes in the cells of the invention may include, for example, the presence of one or more (e.g., one, two, three or four) of the following components: (a) inducing agent (for e.g., an episome maintaining agent for the maintenance of vectors harboring one or more reprogramming genes in cassettes), (b) activating agent (for e.g., dsRNA for activating a different set of reprogramming genes, some of which may, for example, be endogenous within the host cell, or, which may be encoded by a vector), (c) inhibitory agent (for e.g., miRNA, siRNA, antisense molecule, etc., for inhibiting the expression of certain genes), (d) small molecule that affects chromatin methylation status, etc., until the desired level of reprogramming has been achieved, and after which, the presence of these agents can be removed from the media.

Recombinational Cloning

One means by which reprogrammable genes or stem cell markers used to manipulate the stem cell may be assembled into episomal expression vectors is by the use of recombinational cloning. Thus, the invention includes compositions and methods related to recombination cloning and recombination sites, as well as recombination cloning components.

A number of recombinational cloning systems are known. Examples of recombination sites which may be sued in such systems include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

These cloning systems are typically based upon the principle that particular recombination sites will recombine with their cognate counterparts. Nucleci acid molecules of the invention may be designed so as the contain recombination sites of different recombinational cloning systems (e.g., lox sites and att sites). As an example, a nucleic acid molecule of the invention may contain a single lox site and two att sites, wherein the att sites do not recombine with each other.

Recombination sites for use in the invention may be any nucleic acid that can serve as a substrate in a recombination reaction. Such recombination sites may be wild type or naturally occurring recombination sites, or modified, variant, derivative, or mutant recombination sites. Examples of recombination sites for use in the invention include, but are not limited to, phage lambda recombination sites (such as attP, attB, attL, and attR and mutants or derivatives thereof) and recombination sites from other bacteriophage such as phi80, P22, P2, 186, P4 and P1 (including lox sites such as loxP and loxP511). Mutated att sites (e.g., attB 1 10, attP 1 10, attR 1 10 and attL 1 10) are described in U.S. Appl. No. 60/136,744, filed May 28, 1999, and U.S. application Ser. No. 09/517,466, filed Mar. 2, 2000, which are specifically incorporated herein by reference. Other recombination sites having unique specificity (i.e., a first site will recombine with its corresponding site and will not recombine with a second site having a different specificity) are known to those skilled in the art and may be used to practice the present invention. Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g. TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in Escherilica coli. Other suitable recombination sites may be found in U.S. Pat. No. 5,851,808 issued to Elledge and Liu which is specifically incorporated herein by reference. Recombination proteins and mutant, modified, variant, or derivative recombination sites which may be used in the practice of the invention include those described in U.S. Pat. Nos. 5,888,732 and 6,143,557, and in U.S. application Ser. No. 09/438,358 (filed Nov. 12, 1999), based upon U.S. provisional application No. 60/108,324 (filed Nov. 13, 1998), and U.S. application Ser. No. 09/517,466 (filed Mar. 2, 2000), based upon U.S. provisional application No. 60/136,744 (filed May 28, 1999), as well as those associated with the Gateway™ Cloning Technology available from Invitrogen Corp., (Carlsbad, Calif.), the entire disclosures of all of which are specifically incorporated herein by reference in their entireties.

Representative examples of recombination sites which can be used in the practice of the invention include att sites referred to above. Au sites which specifically recombine with other au sites can be constructed by altering nucleotides in and near the 7 base pair overlap region. Thus, recombination sites suitable for use in the methods, compositions, and vectors of the invention include, but are not limited to, those with insertions, deletions or substitutions of one, two, three, four, or more nucleotide bases within the 15 base pair core region (GCTTTTTTATACTAA (SEQ ID NO: 50)), which is identical in all four wild type lambda au sites, attB, attP, attL and attR (see U.S. application Ser. No. 08/663,002, filed Jun. 7, 1996 (now U.S. Pat. No. 5,888,732) and 09/177,387, filed Oct. 23, 1998, which describes the core region in further detail, and the disclosures of which are incorporated herein by reference in their entireties). Recombination sites suitable for use in the methods, compositions, and vectors of the invention also include those with insertions, deletions or substitutions of one, two, three, four, or more nucleotide bases within the 15 base pair core region (GCTTTTTTATACTAA (SEQ ID NO: 50)) which are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

Analogously, the core regions in attB1, attP1, attL1 and attR1 are identical to one another, as are the core regions in attB2, attP2, attL2 and attR2. Nucleic acid molecules suitable for use with the invention also include those which comprising insertions, deletions or substitutions of one, two, three, four, or more nucleotides within the seven base pair overlap region (TTTATAC, which is defined by the cut sites for the integrase protein and is the region where strand exchange takes place) that occurs within this 15 base pair core region (GCTTTTTTATACTAA (SEQ ID NO: 50)).

MultiSite Gateway® technology is described in U.S. Patent Publication No. 2004/0229229 A1, the entire disclosure of which is incorporated herein by reference, and is effective for cloning multiple DNA fragments into one vector without using restriction enzymes. This system can be used to link 1, 2, 3, 4, 5 or more nucleic acid segments, as well as to introduce such segments into vectors (e.g., a single vector). The Gateway® (e.g., MultiSite Gateway®) system allows for combinations of different promoters, DNA elements, and genes to be studied in the same vector or plasmid, for efficient gene delivery and expression. Instead of transfecting multiple plasmids for each gene of interest, a single plasmid carrying different DNA elements, referred to as “an expression cassette” can be studied in the same genomic background.

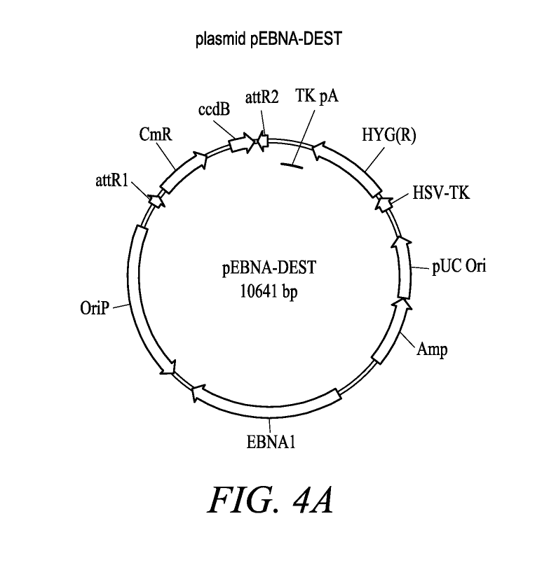

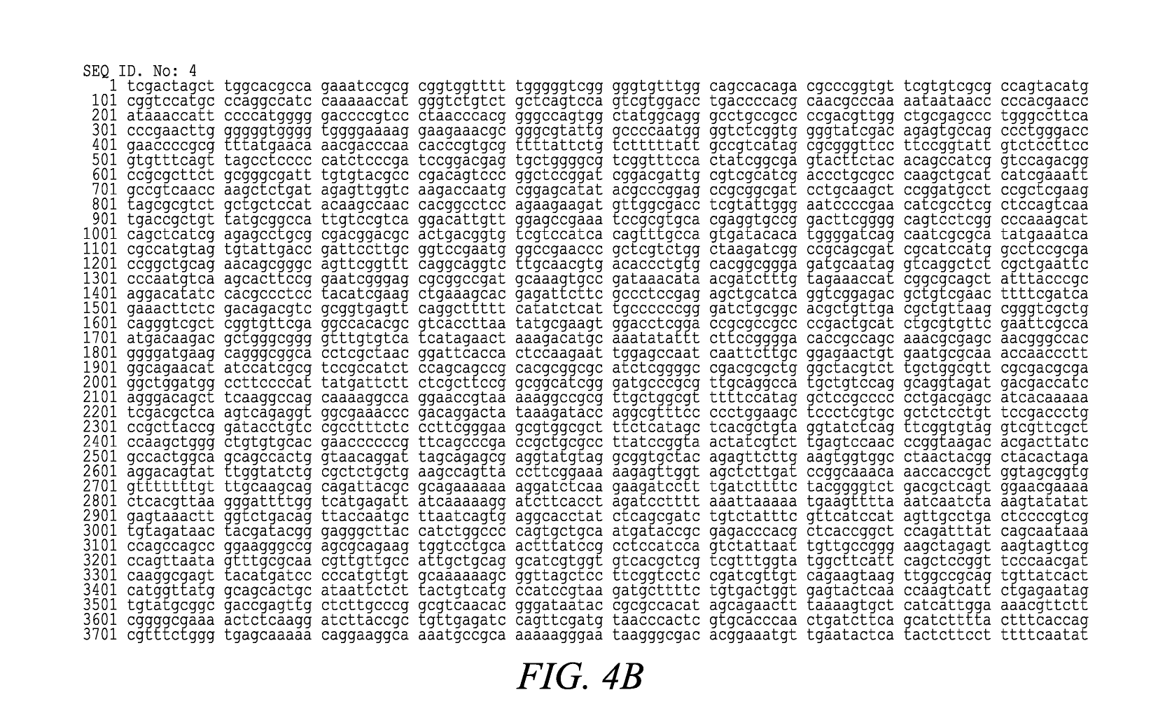

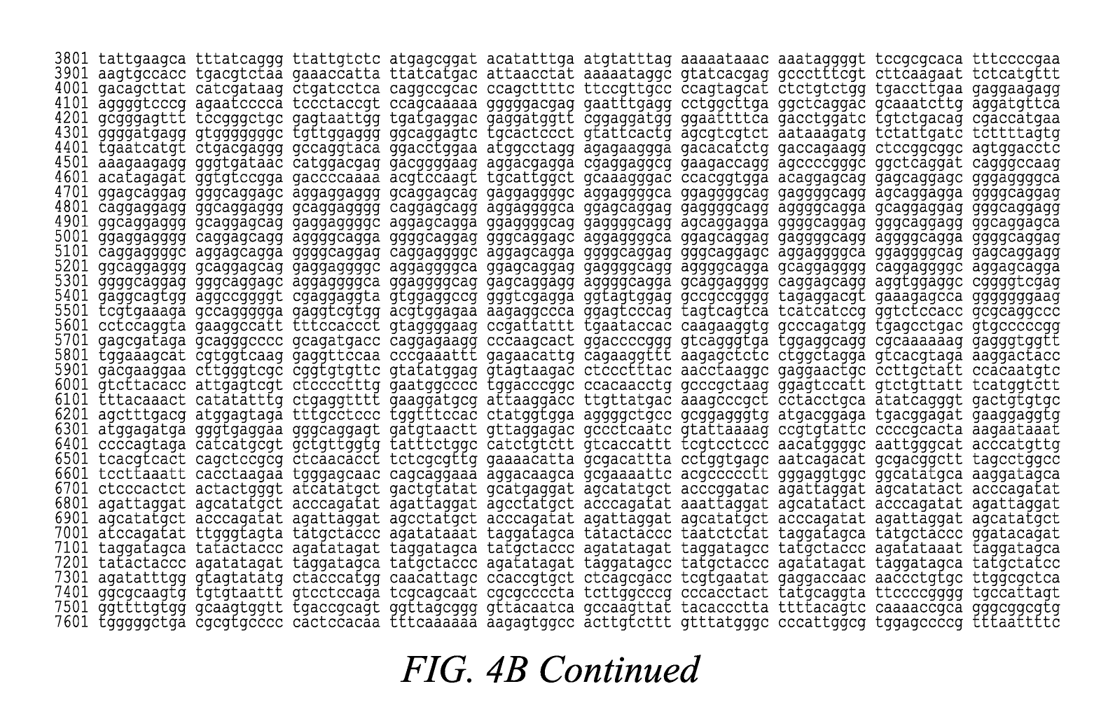

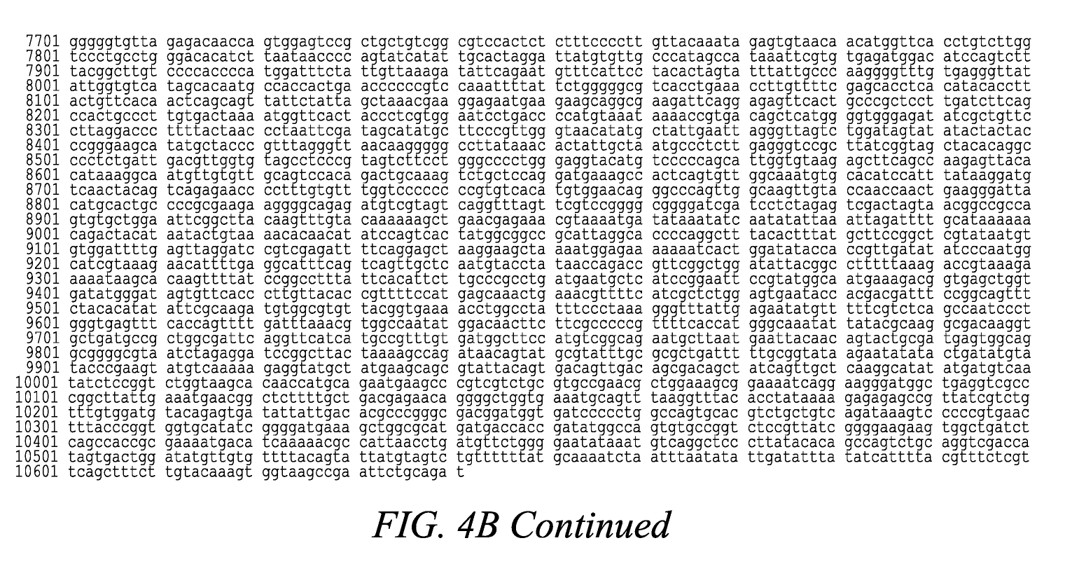

In one embodiment of the invention and by way of example, a plasmid is provided which contains attR1 and attR2 recombination sites. This vector is recombined with a nucleic acid segment which contains a promoter (e.g., an Oct4 promoter) that is flanked by attL1 and attL3 recombination sites and a nucleic acid segment which contains an open reading frame flanked by attR3 and attL2 recombination sites. Upon recombination in the presence of an LR clonase (Invitrogen Corp. Carlsbad, Calif.), the result is the linkage of the promoter to the open reading frame and insertion of the resulting molecule into the plasmid between the attL1 and attL2 recombination sites. As similar example may be found in FIG. 4 of U.S. Patent Publication No. 2004/0229229 A1.

Topoisomerase Mediated Ligation

The present invention also relates to methods of using one or more topoisomerases to generate a recombinant nucleic acid molecules of the invention (e.g., molecules comprising all or a portion of a viral genome such as a viral vector) comprising two or more nucleotide sequences, any one or more of which may comprise, for example, all or a portion of a viral genome. Topoisomerases may be used in combination with recombinational cloning techniques described herein. For example, a topoisomerase-mediated reaction may be used to attach one or more recombination sites to one or more nucleic acid segments. The segments may then be further manipulated and combined using, for example, recombinational cloning techniques.

In one aspect, the present invention provides methods for linking a first and at least a second nucleic acid segment (either or both of which may contain viral sequences and/or sequences of interest) with at least one (e.g., 1, 2, 3, 4, 5, 6, 7, 8, 9, 10, etc.) topoisomerase (e.g., a type IA, type IB, and/or type II topoisomerase) such that either one or both strands of the linked segments are covalently joined at the site where the segments are linked.

A method for generating a double stranded recombinant nucleic acid molecule covalently linked in one strand can be performed by contacting a first nucleic acid molecule which has a site-specific topoisomerase recognition site (e.g., a type IA or a type II topoisomerase recognition site), or a cleavage product thereof, at a 5′ or 3′ terminus, with a second (or other) nucleic acid molecule, and optionally, a topoisomerase (e.g., a type IA, type IB, and/or type II topoisomerase), such that the second nucleotide sequence can be covalently attached to the first nucleotide sequence. As disclosed herein, the methods of the invention can be performed using any number of nucleotide sequences, typically nucleic acid molecules wherein at least one of the nucleotide sequences has a site-specific topoisomerase recognition site (e.g., a type IA, type IB or type II topoisomerase), or cleavage product thereof, at one or both 5′ and/or 3′ termini.

Topoisomerase mediated nucleic acid ligation methods are described in detail in U.S. Patent Publ. No. 2004/0265863 A1, the entire disclosure of which is incorporated herein by reference.

In one aspect of the invention, a detectable or selectable marker may be used. The nucleic acid segment encoding the marker allows one to select for or against a molecule (e.g., a drug resistance marker), or a cell that contains it and/or permits identification of that cell or organism that contains or does not contain the molecule, or the nucleic acid encoding the molecule. Selectable markers can also encode an activity, such as, but not limited to, production of RNA, peptide, or protein, or can provide a binding site for RNA, peptides, proteins, inorganic and organic compounds or compositions and the like. Examples of selectable markers (e.g., negative selectable markers and positive selectable markers) include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as β-lactamase, β-galactosidase, green fluorescent protein (GFP), yellow flourescent protein (YFP), red fluorescent protein (RFP), cyan fluorescent protein (CFP), and cell surface proteins); cameleon chimeras of fluorescent proteins (Miyawaki et al. Nature 1997, vol. 388(6645):882-7 and U.S. Pat. No. 5,998,204 incorporated herein by reference in their entirety); (5) nucleic acid segments that bind products that are otherwise detrimental to cell survival and/or function; (6) nucleic acid segments that otherwise inhibit the activity of any of the nucleic acid segments (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; and/or (11) nucleic acid segments that encode products that either are toxic (e.g., Diphtheria toxin) or convert a relatively non-toxic compound (called “prodrugs”) into a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

In one embodiment, the detectable or selectable marker is a drug resistance (such as antibiotic resistance) gene. The selectable marker may or may not be linked to a differentiation state specific promoter. Drug-resistance may occur at all different levels of drug action and their mechanisms can be classified as being a pre-target event, a drug-target interaction or a post-target event. Common antibiotic resistance selectable markers useful in the invention include, but are not limited to, antibiotics such as ampicillin, tetracycline, kanamycin, bleomycin, streptomycin, blasticidin, hygromycin, neomycin, Zeocin™, and the like.

In some embodiments, the selectable marker may be an auxotrophic genes, which include, for example, hisD, that allows growth in histidine free media in the presence of histidinol. Auxotrophic markers allow cells to synthesize an essential component (usually an amino acid) while grown in media that lacks that essential component.

In one embodiment, selectable markers include fluorescent proteins or membrane tags, which may be used with magnetic beads, cell sorters or other means, to separate cells.

One main purpose of using a fluorescent protein as a selectable marker is to visualize cells, including live visualization of cells. Thus, the selectable marker may enable visual screening of host cells to determine the presence or absence of the marker. For example, a selectable marker may alter the color and/or fluorescence characteristics of a cell containing it. This alteration may occur in the presence of one or more compounds, for example, as a result of an interaction between a polypeptide encoded by the selectable marker and the compound (e.g., an enzymatic reaction using the compound as a substrate). Such alterations in visual characteristics can be used to physically separate the cells containing the selectable marker from those not contain it by, for example, fluorescent activated cell sorting (FACS).

In one aspect of the invention, the invention is applicable to the use of a Lineage Light BacMam system, which allows the identification, enrichment or isolation of any cell type of interest from a mixture of cells. For instance, a lineage-specific promoter may help to identify, label, or separate, specific cell types from a heterogeneous mixture of cells, i.e., differentiated cells that express the lineage-specific driven genes encoded by the vector from other non-expressing cells. In one embodiment where a lineage-specific promoter is used, for instance, a liver specific promoter such as AFP driving the expression of GFP, Lineage Light can be used to identify embryonic stem cells that are differentiating into liver cells. The Lineage light reagent can be directly applied to cells during various stages of differentiation to detect the presence of a cell type of interest.

Constitutive BacMam vectors of the invention may typically be applicable to cases where longer term expression of the Lineage Light is needed, for example to monitor progress of a stem cell to a more mature cell type.

Certain embodiments of the invention include contacting a cell (e.g., stem cell) with a recombinant virus comprising the viral vector that includes (a) an OriP site, (b) optionally, a DNA segment encoding EBNA1; (c) one or more (e.g., one, two, three, etc.) recombination sites (e.g., one or more att sites); and/or (c) at least one selectable marker. Methods of the invention may use, for example, general cell culture and viral infection methods known in the art (e.g., Boyce and Bucher (Baculovirus-mediated gene transfer into mammalian cells): Proc. Natl. Acad. Sci. USA: 93:2348 (1996), incorporated by reference in its entirety). Methods of the invention may also allow cells to live under in vitro conditions such as conventional tissue culture conditions, during which, upon expressing specific genes of interest using the compositions described herein, live cells expressing the specific gene (e.g., a differentiation marker) can be visualized. A purpose of visualizing live cells may be for identification, enrichment or isolation of a particular cell type from a mixture of cells.

To practice methods of the invention for live culture detection, a tissue culture vessel can be inoculated and cells allowed to grow and optionally attach, depending on the cell type. The cell can be allowed to grow, for example for 1 hour to 2 days, 2 hours to 1.5 days, or 4 hours to 1 day. Then medium can be aspirated and a recombinant virus of the invention, for example diluted in a buffer such as PBS, can be applied to the cells for 15 minutes to 72 hours, or in an illustrative embodiment for 2-4 hours, or for 5-60 minutes, or for 15-30 minutes for stem cell or primary cell cultures. After the incubation with virus, the viral infection media can then be replaced with growth media that can include an enhancer, as disclosed herein, for 15 minutes to 8 hours, or from 1-4 hours, or from 1.5-2 hours at 37 C. Cells can then be grown in media and analyzed. In some embodiments, the cell may be allowed to live on a substrate which contains collagen, such as Type I collagen, or rat tail collagen, or on a matrix containing laminin. Implantable versions of such substrates may also be suitable for use in the invention (see, e.g., Hubbell et al., 1995, Bio/Technology 13:565-576 and Langer and Vacanti, 1993, Science 260: 920-925). As an alternative to, or in addition to, allowing cells to live under in vitro conditions, the cells may be allowed to live under in vivo conditions in an animal (e.g., in a human).

Other selection and/or identification may be accomplished by techniques well known in the art. For example, when a selectable marker confers resistance to an otherwise toxic compound, selection may be accomplished by contacting a population of host cells with the toxic compound under conditions in which only those host cells containing the selectable marker are viable. In another example, a selectable marker may confer sensitivity to an otherwise benign compound and selection may be accomplished by contacting a population of host cells with the benign compound under conditions in which only those host cells that do not contain the selectable marker are viable. A selectable marker may make it possible to identify host cells containing or not containing the marker by selection of appropriate conditions.

Multiple selectable markers may be simultaneously used to distinguish various populations of cells. For example, a nucleic acid molecule of the invention may have multiple selectable markers, one or more of which may be removed from the nucleic acid molecule by a suitable reaction. After the reaction, the nucleic acid molecules may be introduced into a host cell population and those host cells comprising nucleic acid molecules having all of the selectable markers may be distinguished from host cells comprising nucleic acid molecules in which one or more selectable markers have been removed. For example, a nucleic acid molecule of the invention may have a blasticidin resistance marker outside a pair of recombination sites and a β-lactamase encoding selectable marker inside the recombination sites. After a recombination reaction and introduction of the reaction mixture into a cell population, cells comprising any nucleic acid molecule can be selected for by contacting the cell population with blasticidin. Optionally, the desired cells can be physically separated from undesirable cells, for example, by FACS.

One use of such a system is to identify or select for cells entering a specific state of differentiation. Many different combinations of developmentally related promoters with reporter genes, selection markers and regulatory genes can be envisaged. In some embodiments, a membrane tag may be operably linked to a promoter to allow selection of differentiated cells from culture using magnetic beads, FACS or other means. The invention also includes methods for using inserted genetic elements to produce cells with particular properties, methods for the regulation of gene expression by the use of RNAi molecules, methods for the regulation of cell differention, methods for selecting cells based on differentiation state, and methods for producing cells with limited differentiation potential.

Preparation of Stem Cells

Methods of the invention include those directed to preparation of cells (e.g., stem cells). Exemplary methods include those related to the introduction into cells (e.g., stem cells) at least one episomal nucleic acid construct described in the invention, comprising at least the EBNA1 expressing DNA fragment, optionally a tet repressor fragment, at least one OriP containing vector, and at least one recombinational cloning (e.g., Gateway®) recombination site into which any gene or genes of interest can be cloned. In some aspects of the invention, cells (e.g., stem cells) can be maintained in a desired state of differentiation, by use of the regulatable (e.g., inducible or repressible) promoters. In the presence of the inducing agent, for example, tetracycline, the cell will express the EBNA1 protein that binds to the OriP to facilitate the retention and replication of all the OriP containing vectors, ensuring expression of genes introduced thereby during the reprogramming period. Once reprogrammed cells or induced pluripotent cells (iPCs) are obtained, the tetracycline is removed resulting in the repression of the EBNA1 protein by the tet repressor, and the episomal plasmids will not be maintained after couple of rounds of cell division. EBNA1 protein expression and subsequently, replication of the episomal plasmids get diluted out since this system does not integrate the vector components into the cell's genome. Therefore, gene expression is only sustained during the period required for reprogramming, allowing for the loss of ectopic genes after removal of the inducer (tetracycline).

Maintenance and expansion of embryonic stem cells is described in U.S. Pat. No. 5,453,357. In some aspects of the invention, stem cells can be maintained in a desired state of differentiation, by the use of differentiation state or cell lineage associated promoters that are operably linked to an antibiotic resistance gene. A differentiation state associated promoter is one in which the function of the promoter is tied to the differentiation state of the cell. When the cell begins to differentiate, the function of the promoter decreases and the expression of linked antibiotic resistance gene is reduced and the cell becomes susceptible to the appropriate antibiotic. A cell lineage associated promoter is one in which the promoter displays differential activity in a specific cell lineage. A cell lineage associated promoter may not be functional or will have different activity in cells of a different lineage. This same principal can be used to select cells (e.g., stem cells) that move down a particular differentiation pathway where an antibiotic resistance gene is operably linked to a promoter which becomes active only when the cell (e.g., stem cell) differentiates along the desired lineage pathway. The appropriate antibiotic can then be used to eliminate cells which have differentiated down the wrong pathway or which belong to the wrong lineage.

In some embodiments, cells (e.g., stem cells) may be engineered to contain multiple differentiation state or lineage associated genes each operably linked to a unique promoter, and further, each gene associated with a unique antibiotic resistance profile. This allows selection of cells (e.g., stem cells) that have a variety of antibiotic resistance profiles depending on the differentiation pathway they follow. In some instances all of the promoters may remain transcriptionally active so that the cells (e.g., stem cells) will remain resistant to all of the antibiotics. In other instances, some promoters may remain or become transcriptionally active in one differentiation pathway, but not in another pathway. This will result in specific patterns of gene expression for specific differentiation pathways and allow for specifically selecting cells (e.g., stem cells) which follow a desired differentiation pathway.

The invention may also be used to induce in vivo cell (e.g., stem cell) or progenitor cell mobilization, migration, integration, proliferation and differentiation.

Stem cells may be pluripotent, that is, they may be capable of giving rise to a plurality of different differentiated cell types. In some cases stem cells may be totipotent, that is, they may be capable of giving rise to all of the different cell types of the organism that they are derived from. The invention is applicable to progenitor, totipotent, pluripotent or multipotent stem cells.

In some embodiments, the invention is used to genetically modify adult cells (e.g., stem cells). Adult stem cells are known to occur in a number of locations in the animal body. Adult stem cells may be those from any of organs and tissues in which stem cells are present. Examples include stem cells from bone marrow, haematopoietic system, neuronal system, brain, muscle stem cells or umbilical cord stem cells. Stem cells may in particular be bone marrow stromal stem cells, neuronal stem cells or haematopoietic stem cells.

In some embodiment, cells (e.g., stem cells) used in the practice of the invention may be human cells (e.g., stem cells). Alternatively, cells (e.g., stem cells) may be from a non-human animal and in particular from a non-human mammal. Cells (e.g., stem cells) may be those of a domestic animal or an agriculturally important animal. An animal may, for example, be a sheep, pig, cow, horse, bull, or poultry bird or other commercially-farmed animal. An animal may be a dog, cat, or bird and in particular from a domesticated animal. An animal may be a non-human primate such as a monkey. For example, a primate may be a chimpanzee, gorilla, or orangutan. Cells (e.g., stem cells) used in the practice of the invention may be rodent stem cells. For example, cells (e.g., stem cells) may be from a mouse, rat, or hamster.

In one embodiment, cells (e.g., stem cells) used in the practice of the invention may be plant cells (e.g., stem cells). Stem cells are known to occur in a number of locations in the seed and developing or adult plant. Stem cells genetically modified or obtained in the present invention may be those from any of the tissues in which stem cells are present. Examples include stem cells from the apical or root meristems. In one embodiment, the stem cells are from an agriculturally important plant. Plants may, for example, be maize, wheat, rice, potato, an edible fruit-bearing plant or other commercially farmed plant.

In some cases, genetically modified cells (e.g., stem cells) may be intended to treat a subject, or in the manufacture of medicaments. In such cases, cells (e.g., stem cells) may be from the intended recipient. In other cases, cells (e.g., stem cells) may originate from a different subject, but be chosen to be immunologically compatible with the intended recipient. In some cases cells (e.g., stem cells) may be from a relation of the intended recipient such as a sibling, half-sibling, cousin, parent or child, and in particular from a sibling. Cells (e.g., stem cells) may be from an unrelated subject who has been tissue typed and found to have a immunological profile which will result in no immune response or only a low immune response from the intended recipient which is not detrimental to the subject. However, in many cases the cells (e.g., stem cells), may be from an unrelated subject as the invention may be used to render the stem cell immunologically compatible with the intended recipient. For example, cells (e.g., stem cells) and the recipient may or may not have a histocompatible haplotypes (e.g., HLA haplotypes).

Cell (e.g., stem cell) lines are generally cell (e.g., stem cell) populations that have been isolated from an organism and maintained in culture. Thus the invention may be applied to cell (e.g., stem cell) lines including adult, fetal, embryonic, neonatal or juvenile stem cell lines. Cell (e.g., stem cell) lines may be clonal i.e., they may have originated from a single cell (e.g., stem cell). In one embodiment, the invention may be applied to existing stem cell lines, particularly to existing embryonic and fetal stem cell lines. In other cases the invention may be applied to a newly established cell (e.g., stem cell) line.

Cells (e.g., stem cells) used in the practice of the invention may be an existing stem cell line. Examples of existing cell (e.g., stem cell) lines which may be used in the invention include the human embryonic stem cell line provided by Geron (Menlo Park, Calif.) and the neural stem cell line provided by ReNeuron (Guildford, United Kingdom). In some embodiments, the cell (e.g., stem cell) line may be one which is a freely available stem cell, access to which is open. Additional sources for cell (e.g., stem cell) lines include but are not limited to BresaGen Inc. of Australia; CyThera Inc.; the Karolinska Institute of Stockholm, Sweden; Monash University of Melbourne, Australia; National Centre for Biological Sciences of Bangalore, India; Reliance Life Sciences of Mumbai, India; Technion-Israel Institute of Technology of Haifa, Israel; the University of California at San Francisco; Goteborg University of Goteborg, Sweden; and the Wisconsin Alumni Research Foundation; and Cellartis (Sweden); and ESI (Singapore).

Reference herein to stem cell generally includes the embodiment mentioned also being applicable to stem cell lines unless, for example, it is evident that target cells are freshly isolated stem cells or stem cells are resident stem cells in vivo. The invention is applicable to freshly isolated stem cells and also to cell populations comprising stem cells. The invention may also be used to control the differentiation of stem cells in vivo.

Methods for isolating particular types of cells (e.g., stem cells) are well known in the art and may be used to obtain cells (e.g., stem cells) suitable for use in the invention. Such methods may, for example, be used to recover cells (e.g., stem cells) from intended recipients of medicaments of the invention. Cell surface markers characteristic of cells (e.g., stem cells) may be used to isolate the stem cells, for example, by cell sorting. Cells (e.g., stem cells) may be obtained from any of the types of subjects mentioned herein and in particular, from those suffering from any of the disorders mentioned herein.

In some embodiments cells (e.g., stem cells) may be obtained by using the methods of the invention to reverse the differentiation of differentiated cells to give stem cells. In particular, differentiated cells may be recovered from a subject, treated in vitro in order to produce stem cells, the cells (e.g., stem cells) obtained may then be manipulated as desired and differentiated before (and/or after) return to the subject. As stem cells typically represent a very small minority of the cells present in an individual such an approach may be preferable. It may also mean that stem cells are more easily derivable from specific individuals and may eliminate the need for embryonic stem cells. In addition, typically such an approach will be less labor intensive and expensive than methods for isolating stem cells themselves. In some cases, stem cells may be isolated from a subject, differentiated in vitro and then returned to the same subject.

In many embodiments stem cells may be any of the types of stem cells mentioned herein and may be in any of the organisms mentioned herein. Target stem cells may be present in any of the organs, tissues or cell populations of the body in which stem cells exist, including any of those mentioned herein. Target stem cells will typically be resident stem cells naturally occurring in the subject, but in some cases stem cells produced using the methods of the invention may be transferred into the subject and then induced to differentiate by transfer of RNA.

Various techniques for isolating, maintaining, expanding, characterizing and manipulating stem cells in culture are known and may be employed. In a preferred embodiment, genetic modifications may be introduced into genomes of stem cells. Stem cells lend themselves to such manipulation as clonal lines can be established and readily screened using techniques such as PCR or Southern blotting.

In some instances cells (e.g., stem cells) may originate from an individual or animal with a genetic defect. Methods described herein may be used to make modifications to correct or ameliorate the defect. For example, a functional copy of a missing or defective gene may be introduced into the genome of the cell. In a particular embodiment, differentiated cells may be obtained from an individual with a genetic defect, stem cells obtained from the differentiated cells using the methods disclosed herein, the genetic defect corrected or ameliorated and then either the stem cells or differentiated cells obtained from them will be used for treating the original subject or in the manufacture of medicaments for treating the original subject.

Expression vectors contemplated by the invention may contain additional nucleic acid fragments such as control sequences, marker sequences, selection sequences and the like as discussed below.

In one aspect of the present invention, at least one recombinational cloning (e.g., MultiSite Gateway®) cloning site for cloning in at least one desired “gene expression cassette” may be identified in a cell (e.g., stem cell) of interest, while the inducing agent is present.

In many embodiments of the present invention, a collection of useful genetic elements or a genetic toolbox is created. Components of the toolbox may comprise transcriptional promoters and reporters. Suitable promoters include, but are not limited to, constitutive viral, human and mouse tissue-specific, regulatable promoters. Suitable reporters include, but are not limited to, green fluorescent protein (GFP) variants, β-lactamase, lumio, magnetic resonance imaging (MRI), and positron emission tomography (PET) contrasting proteins. Additional components of the toolbox could include other elements useful for genomic engineering such as toxin genes, recombination sites, internal ribosomal entry segment (IRES) sequences, etc.

The elements of the toolbox may first be placed into entry clones. The first step of preparing an entry clone may be to amplify the genetic element by polymerase chain reaction (PCR) followed by cloning into a TA or any other cloning vector. General procedures for PCR are taught in MacPherson et al., PCR: A Practical Approach, (IRL Press at Oxford University Press, (1991)). PCR conditions for each application reaction may be empirically determined. A number of parameters influence the success of a reaction. Among these parameters are annealing temperature and time, extension time, Mg²⁺ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

The final expression vector is produced by recombining entry clones containing the desired genetic elements with a destination vector containing appropriate attR sites and a selection marker. Such procedures can be used to produce a simple expression vector with, for example two elements, a promoter and a gene to be expressed, or more complex expression vectors with, three, four, five, seven, ten, twelve, fifteen, twenty, thirty, fifty, seventy-five, one hundred, two hundred, etc. genetic elements. Intermediate destination vectors may be used prepare expression vectors with large numbers of genetic elements as outlined in Attachments A through P.

A variety of expression vectors are suitable for use in the practice of the present invention. In general, an expression vector will have one or more of the following features: a promoter, promoter-enhancer sequences, a selection marker sequence, an origin of replication, an inducible element sequence, a repressible element sequence, an epitope-tag sequence, and the like.

Other exemplary eukaryotic promoters, or combinations of DNA segments from different promoters may also be used in the invention, and include, but are not limited to, the CMV (cytomegalovirus) promoter, the CMV/inducible operon promoter (for example, the CMV/TO promoter, where parts of the CMV promoter and the Tet operon promoter is combined), mouse metallothionein I gene promoter (Hamer et al., J. Mol. Appl. Gen. 1:273-288, (1982)); Herpes virus TK promoter (McKnight, Cell 31:355-365, (1982)); the SV40 early promoter (Benoist et al., Nature (London) 290:304-310, (1981)); the yeast gall gene sequence promoter (Johnston et al., Proc. Natl. Acad. Sci. (USA) 79:6971-6975, (1982)); Silver et al., Proc. Natl. Acad. Sci. (USA) 81:5951-59SS, (1984), the EF-1 promoter, Ecdysone-responsive promoter(s), tetracycline-responsive promoter, and the like. Promoters also include tissue-specific promoters to allow for tissue specific expression.

Exemplary promoters for use in the present invention may be selected such that they are functional in a particular cell or tissue type into which they are introduced.

A further element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, colE1 plasmid origin, 2μ and ARS (both useful in yeast systems), sf1, SV40, EBV OriP (useful in mammalian systems), and the like.

Epitope tags may be necessary in certain cases. These are short peptide sequences that when tagged to a desired gene, is expressed as a fusion protein comprising the desired protein sequence with the epitope tag, and may help to easily identify or purify (using an antibody bound to a chromatography resin) the fusion protein. The presence of the epitope tags on proteins may be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

A further useful element in an expression vector is a multiple cloning site or polylinker. Synthetic DNA encoding a series of restriction endonuclease recognition sites is inserted into a plasmid vector, for example, downstream of the promoter element. These sites are engineered for convenient cloning of DNA into the vector at a specific position.

The foregoing elements can be combined to produce expression vectors suitable for use in the methods of the invention. Those of skill in the art would be able to select and combine the elements suitable for use in their particular system in view of the teachings of the present specification.

Individual elements of the genetic toolbox, including but not limited to, cloned genetic elements, entry clones containing individual genetic elements, destination vectors, accessory products such as selection antibiotics, competent cells, accessory purification tools/kits like plasmid purification kits, transfection reagents, expression clone construction kits, etc. of the present invention can be formulated into kits. Components of such kits can include, but are not limited to, containers, instructions, solutions, buffers, disposables, and hardware.

Cells (e.g., stem cells) modified by the methods of the present invention can be maintained under conditions that, for example, (i) keep them alive but do not promote growth, (ii) promote growth of the cells, and/or (iii) cause the cells to differentiate or dedifferentiate. Cell culture conditions are typically permissive for the action of the reprogramming genes in the cells, that is, in the presence of an inducing (for e.g., episome maintaining agent), activating (for e.g., dsRNA) or inhibitory (for e.g., miRNA, siRNA, antisense molecules, etc.) agent until the desired level of reprogramming has been achieved, upon which, presence of the inducing or activating or inhibitory agent is removed from the media. For a given cell, cell-type, tissue, or organism, culture conditions are known in the art. These conditions include, but are not limited to, the use of defined media, serum-free medium, culture of cells in feeder-free culturing conditions, and matrices for the maintenance of stem cells in culture.

Transgenic Non-Human Animals

In another embodiment, the present invention comprises transgenic nonhuman transgenic animals whose genomes have been modified by employing the methods and compositions of the invention. Transgenic animals may be produced employing the methods of the present invention to serve as a model system for the study of various disorders and for screening of drugs that modulate such disorders.

A “transgenic” animal may be a genetically engineered animal, or offspring of genetically engineered animals. A transgenic animal usually contains material from at least one unrelated organism, such as, from a virus. The term “animal” as used in the context of transgenic organisms means all species except human. It also includes an individual animal in all stages of development, including embryonic and fetal stages. Farm animals (e.g., chickens, pigs, goats, sheep, cows, horses, rabbits and the like), rodents (such as mice), and domestic pets (e.g., cats and dogs) are included within the scope of the present invention. In some embodiments, the animal may be a mouse or a rat.

The term “chimeric” animal may be an animal in which any heterologous gene may be found, or in which, a heterologous gene may be expressed, in some, but not all cells of the animal.

The term transgenic animal also includes a germ cell line transgenic animal. A “germ cell line transgenic animal” may be a transgenic animal in which the genetic information provided by the method of the invention may be taken up and incorporated into a germ line cell, therefore conferring the ability to transfer the information to an offspring. If such offspring, in fact, possess some or all of that information, then they, too, are transgenic animals.

Methods of generating transgenic plants and animals are known in the art and can be used in combination with the teachings of the present application.

In one embodiment, a transgenic animal of the present invention may be produced by introducing into a single cell embryo at least one episomal nucleic acid construct described in this invention, comprising at least the EBNA1 expressing DNA fragment, OriP and recombinational cloning (e.g., MultiSite Gateway®) recombination sites into which any gene or genes of interest can be cloned. The DNA of germ line cells of the mature animal and is inherited in normal Mendelian fashion. In the presence of an inducing agent, for example, tetracycline, it will result in the expression of the EBNA1 protein that binds to the OriP to facilitate the retention and replication of the OriP containing vectors, ensuring its expression during the reprogramming period. Once reprogrammed cells or induced pluripotent cells (iPCs) are obtained, the tetracycline is removed resulting in the repression of the EBNA1 protein by the tet repressor, and the episomal plasmids will not be maintained after couple of rounds of cell division. Since this system does not integrate the vector components into the cell's genome, it only sustains gene expression during the period required for reprogramming, allowing for the loss of ectopic genes after removal of the inducer (tetracycline).

By way of example only, to prepare a transgenic mouse, female mice are induced to superovulate. After being allowed to mate, the females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice. (See Hammer, et al., Cell 63:1099-1112, (1990)). Rodents suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan, et al., supra). Microinjection procedures for fish, amphibian eggs and birds are detailed in Houdebine and Chourrout, Experientia 47:897-905, (1991)). Other procedures for introduction of DNA into tissues of animals are described in U.S. Pat. No. 4,945,050 (Sandford et al., Jul. 30, (1990)).

Pluripotent or multipotent stem cells derived from the inner cell mass of the embryo and stabilized in culture can be manipulated in culture to incorporate nucleic acid sequences employing invention methods. A transgenic animal can be produced from such cells through injection into a blastocyst that is then implanted into a foster mother and allowed to come to term.

Methods for the culturing of stem cells, and the introduction of DNA into stem cells include methods such as transfection (e.g.: transient or stable), peptide delivery, electroporation, calcium phosphate/DNA precipitation, microinjection, liposome fusion, retroviral infection, and the like are also are well known to those of ordinary skill in the art. The subsequent production of transgenic animals from these stem cells is well known in the art. See, for example, Teratocarcinomas and Embryonic Stem Cells, A Practical Approach, E. J. Robertson, ed., IRL Press, 1987). Reviews of standard laboratory procedures for microinjection of heterologous DNAs into mammalian (mouse, pig, rabbit, sheep, goat, cow) fertilized ova include: Hogan et al., Manipulating the Mouse Embryo (Cold Spring Harbor Press 1986); Krimpenfort et al., (1991), Bio/Technology 9:86; Palmiter et al., (1985), Cell 41:343; Kraemer et al., Genetic Manipulation of the Early Mammalian Embryo (Cold Spring Harbor Laboratory Press 1985); Hammer et al., (1985), Nature, 315:680; Purcel et al., (1986), Science, 244:1281; Wagner et al., U.S. Pat. No. 5,175,385; Krimpenfort et al., U.S. Pat. No. 5,175,384, the respective contents of which are incorporated by reference.

One embodiment of the procedure is to inject targeted embryonic stem cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (see Houdebine and Chourrout, supra; Purcel, et al., Science 244:1281-1288, (1989); and Simms, et al., Bio/Technology 6:179-183, (1988)). Animals carrying the transgene can be identified by methods well known in the art, e.g., by dot blotting or Southern blotting.

The term transgenic as used herein additionally includes any organism whose genome has been altered by in vitro manipulation of the early embryo or fertilized egg or by any transgenic technology to induce a specific gene knockout. The term “gene knockout” as used herein, may be the targeted disruption of a gene in vivo with loss of function that has been achieved by use of the invention vector. In one embodiment, transgenic animals having gene knockouts are those in which the target gene has been rendered nonfunctional by an insertion targeted to the gene to be rendered non-functional by targeting a pseudo-recombination site located within the gene sequence.

Treatment of Disease and Disorders

Reprogramming may be done for any reason, for example, to achieve a more differentiated status of a cell, or to achieve a more stem-like state from a somatic stage, or to achieve a more embryonic-, fetal-, or neonatal-stem cell like state, or to achieve a more non-cancerous state, or a more disease-free state, etc. The ability to reprogram somatic cells, including adult stem cells into an ESC-like state is an emerging field which is opening a new area for creating patient-specific pluripotent cells useful in disease research and cell replacement therapies.

Whether a particular cell has been reprogrammed may be determined by identifying expression of specific cell-markers associated with the reprogrammed state, for instance, identification of embryonic or fetal cell markers, reduction in expression of a cancer marker, reduction in expression of a disease marker, reduction in expression of a damaged cell marker (for e.g.; damaged lung epithelial cell in lung cancer), etc. For instance, unique expression markers may be used to characterize various stem cell populations such as CD34, CD133, ABCG2, Sca-1, etc. for hematopoietic stem cells; STRO-1, etc. for mesenchymal/stromal stem cells; nestin, PSA-NCAM, p75 neurotrophin R (NTR), etc. for neural stem cells. Markers may include the expression (or upregulation) of new peptides or proteins not expressed in the previous state, like a new receptor, a new growth factor, a new hormone (e.g., steroid or peptide), a new structural protein, etc. some or all of which may be associated with a more rejuvenated, repaired or better functional state than the previous injured, diseased or cancerous state. In cancer, markers that may be associated may be expression or upregulation of some tumor suppressor markers such as p10, p53, p16, p63, etc.

Reprogramming of any cell, including stem cells, somatic cells, cancer cells, diseased cells, or normal cells, may be achieved using the compositions and methods described herein.

An embodiment of the invention comprises a method of treating a disorder in a subject in need of such treatment. In one embodiment of the method, a stem cell of the subject has a regulatable (e.g., inducible) episomal vector and reprogrammable genes that are expressed until the inducible agent is present. An episomal expression vector containing one or more genes related to treatment of the condition is then introduced into the cell and maintained with the inducing agent so that expression of the genes occur and reprogramming of the stem cell occurs. After reprogramming, the inducing agent is no longer needed, expression of the gene may no longer be needed, and the reprogrammed stem cell is then reintroduced into the subject. Subjects treated using the methods of the invention include both humans and non-human animals. Such methods utilize the constructs, compositions and methods of the present invention.

Expression vectors useful in such embodiments will often comprise one or more nucleic acid fragments of interest which may contain genes or portions of genes of interest, and/or regulatory nucleic acid molecules like small RNAs, e.g.: dsRNA, RNAᵢ etc. Among the nucleic acid fragments of interest for use in this embodiment, include, therapeutic genes and/or small RNAs to control regions such as promoters and/or enhancers or portions of the gene itself. The choice of nucleic acid sequence will depend on the nature of the disorder to be treated. For example, a nucleic acid construct intended to treat hemophilia B, which is caused by a deficiency of coagulation factor IX, may comprise a nucleic acid fragment encoding functional factor IX. A nucleic acid construct intended to treat obstructive peripheral artery disease may comprise nucleic acid fragments encoding proteins that stimulate the growth of new blood vessels, such as, for example, vascular endothelial growth factor, platelet-derived growth factor, and the like. Those of skill in the art would readily recognize which nucleic acid fragments of interest would be useful in the treatment of a particular disorder.

The invention thus includes compositions and methods for cell reprogramming, including stem cells, somatic cells, damaged cells, etc. and such reprogrammed and/or rejuvenated cells may be used to treat or alleviate the respective disorder or condition. Diseases/conditions include, but are not limited to, cancer treatment, infectious diseases, tissue remodeling, aging, tissue repair, sports injury or other physical injuries (e.g., bone healing and use of chondrocyte stem cultures), burn injury (e.g., for regeneration of skin), chemical injury, allergic injuries, light damage (e.g., retinal damage of eye), hypoxic injuries (e.g., ischemic damage of heart cells), pollution damage (e.g., smoke (cigarette or toxic fumes) damage of lung tissue), monogenic disorders, acquired disorders, and the like. Exemplary monogenic disorders include ADA deficiency, cystic fibrosis, familial-hypercholesterolemia, hemophilia, chronic granulomatous disease, Duchenne muscular dystrophy, Fanconi anemia, sickle-cell anemia, Gaucher's disease, Hunter syndrome, X-linked SCID, and the like.

Infectious diseases treatable by employing the methods of the invention include infection with various types of virus including human T-cell lymphotropic virus, influenza virus, papilloma virus, hepatitis virus, herpes virus, Epstein-Bar virus, immunodeficiency viruses (HIV, and the like), cytomegalovirus, and the like. Also included are infections with other pathogenic organisms such as Mycobacterium Tuberculosis, Mycoplasma pneumoniae, and the like or parasites such as Plasmodium falciparum, and the like.

The term “acquired disorder” as used herein may be a non-congenital disorder. Such disorders are generally considered more complex than monogenic disorders and may result from inappropriate or unwanted activity of one or more genes. Examples of such disorders include peripheral artery disease, rheumatoid arthritis, coronary artery disease, and the like.

A particular group of acquired disorders treatable by employing the methods of the invention include various cancers, including both solid tumors and hematopoietic cancers such as leukemias and lymphomas. Solid tumors that are treatable utilizing the invention method include carcinomas, sarcomas, osteomas, fibrosarcomas, chondrosarcomas, and the like. Specific cancers include breast cancer, brain cancer, lung cancer (non-small cell and small cell), colon cancer, pancreatic cancer, prostate cancer, gastric cancer, bladder cancer, kidney cancer, head and neck cancer, and the like.

Kits

In another aspect, the invention provides kits that may be used in conjunction with methods the invention. Kits according to this aspect of the invention may comprise one or more containers, which may contain one or more components selected from the group consisting of one or more nucleic acid molecules (e.g., one or more nucleic acid molecules comprising one or more recombination sites) of the invention, one or more primers, the molecules and/or compounds of the invention, one or more polymerases, one or more reverse transcriptases, one or more recombination proteins (or other enzymes for carrying out the methods of the invention), one or more cell (e.g., host cell), one or more buffers, one or more detergents, one or more restriction endonucleases, one or more nucleotides, one or more terminating agents (e.g., ddNTPs), one or more transfection reagents, pyrophosphatase, and the like.

A wide variety of nucleic acid molecules can be used with the invention. Further, due to the modularity of the invention, these nucleic acid molecules can be combined in wide range of ways. Examples of nucleic acid molecules that can be supplied in kits of the invention include those that contain promoters, signal peptides, enhancers, repressors, selection markers, transcription signals, translation signals, primer hybridization sites (e.g., for sequencing or PCR), recombination sites, restriction sites and polylinkers, sites that suppress the termination of translation in the presence of a suppressor tRNA, suppressor tRNA coding sequences, sequences that encode domains and/or regions (e.g., 6 His tag) for the preparation of fusion proteins, origins of replication, telomeres, centromeres, and the like.

Similarly, libraries (e.g., libraries derived from stem cells, such as stem cell cDNA libraries) can be supplied in kits of the invention. These libraries may be in the form of replicable nucleic acid molecules or they may comprise nucleic acid molecules that are not associated with an origin of replication. As one skilled in the art would recognize, the nucleic acid molecules of libraries, as well as other nucleic acid molecules that are not associated with an origin of replication, either could be inserted into other nucleic acid molecules that have an origin of replication or would be an expendable kit component.

Further, in some embodiments, libraries supplied in kits of the invention may comprise at least two components: (1) the nucleic acid molecules of these libraries and (2) 5′ and/or 3′ recombination sites. In some embodiments, when the nucleic acid molecules of a library are supplied with 5′ and/or 3′ recombination sites, it will be possible to insert these molecules into a vector, which also may be supplied as a kit component, using recombination reactions. In other embodiments, recombination sites can be attached to the nucleic acid molecules of the libraries before use (e.g., by the use of a ligase, which may also be supplied with the kit). In such cases, nucleic acid molecules that contain recombination sites or primers that can be used to generate recombination sites may be supplied with the kits.

Kits of the invention may contain a nucleic acid molecule as described herein. One example of such a molecule is a plasmid vector described in Attachment B. Further, a kit of the invention may contain only a single nucleic acid molecule in a container, wherein the container (e.g., a box) is designed for shipment via the mail of other suitable carrier. Product literature (see, e.g., Attachment B) may also be included in kits of the invention. Thus, while kits of the invention may contain many components, many kits will be composed of just three items: (1) a nucleic acid molecule, (2) product literature, and (3) a container which holds (1) and (2). Of course, the nucleic acid molecule (i.e., kit component (1)), will generally be in separate container that fits into container (3).

Kits of the invention may also comprise one or more topoisomerase proteins and/or one or more nucleic acids comprising one or more topoisomerase recognition sequence. In many instances, topoisomerase proteins, when present, will be bound to nucleic acids.

Suitable topoisomerases include Type IA topoisomerases, Type IB topoisomerases and/or Type II topoisomerases. Suitable topoisomerases include, but are not limited to, poxvirus topoisomerases, including vaccinia virus DNA topoisomerase I, E. coli topoisomerase III, E. coli topoisomerase I, topoisomerase III, eukaryotic topoisomerase II, archeal reverse gyrase, yeast topoisomerase III, Drosophila topoisomerase III, human topoisomerase III, Streptococcus pneumoniae topoisomerase III, bacterial gyrase, bacterial DNA topoisomerase IV, eukaryotic DNA topoisomerase II, and T-even phage encoded DNA topoisomerases, and the like. Suitable recognition sequences have been described above.

One or more buffers (e.g., one, two, three, four, five, eight, ten, fifteen) may be supplied in kits of the invention. These buffers may be supplied at a working concentrations or may be supplied in concentrated form and then diluted to the working concentrations. These buffers will often contain salt, metal ions, co-factors, metal ion chelating agents, etc. for the enhancement of activities of the stabilization of either the buffer itself or molecules in the buffer. Further, these buffers may be supplied in dried or aqueous forms. When buffers are supplied in a dried form, they will generally be dissolved in water prior to use.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

Kits of the invention may comprise one or more pages of written instructions for carrying out the methods of the invention. For example, instructions may comprise methods steps necessary to carryout recombinational cloning of an ORF provided with recombination sites and a vector also comprising recombination sites and optionally further comprising one or more functional sequences.

The following examples are intended to illustrate, but not limit, certain embodiments of the invention. One skilled in the art will understand that various modifications are readily available and can be performed without substantial change in the way the invention works. All such modifications are specifically intended to be within the scope of the invention claimed herein.

### EXAMPLES

### Example 1

### MultiSite Gateway® Episomal Plasmid Vector Delivery Systems

Epstein Ban virus based episomal plasmid vectors have been successfully used to stably express genes of interest in multiple types of cells both in vitro and in vivo {Belt et al., Gene, 84: 407-417, (1989); James et al., Mutant Res., 220: 169-185, (1989); Mazda et al., Curr. Gene Ther, 2: 379-392, (2002); Stoll et al., Mol. Ther, 4: 122-129, (2001); Van Craenenbroeck et al., Eur J Biochem, 267: 5665-5678, (2000); Wade-Martins et al., Nuc. Acid Res., 27: 1674-1682, (1999). Maintenance of these vectors in primate cells requires two important factors, the Epstein-Ban virus nuclear antigen (EBNA1) and the latent origin of replication OriP. The ability of these vector systems to support episomal maintenance of large genomic fragments makes them appealing for expression of transgenes in cells Van Craenenbroeck et al., Eur J Biochem, 267: 5665-5678, (2000).

Novel episomal plasmid gene delivery vectors were built from components derived from the Epstein-Ban virus and detailed methods for such vector construction are described in Thyagarajan, B. et al., Regenerative Medicine, 4 (2): 239-250, (2009), disclosure of which is hereby incorporated by reference in its entirety. Briefly, the pCEP4 (Invitrogen) vector shown in FIG. 1 (SEQ ID NO.: 1), which contains the EBNA1 expression cassette and the OriP element (origin of replication) on a single plasmid, was adapted to enable MultiSite Gateway® assembly (Invitrogen). This further enables rapid cloning of multiple expression cassettes of interest, each of which can contain different promoters and/or reporters in one step. Thus, expression genes can be stably maintained and expressed as episomes in cells, for e.g., human embryonic stem cells, using a single vector system. This method is also useful in generating stable cell lines expressing the genes introduced via these episomes. If the EBNA gene is expressed via a constitutive promoter, EBNA expression is stable, and expression of genes from the expression cassette is constitutive. On the other hand, if the EBNA gene is expressed via an inducible promoter, then EBNA expression is inducible with the inducible agent, and correspondingly, expression of genes from the expression cassette takes place only in the presence of the inducible agent. In addition, gene expression can be targeted to specific cell types, or cell lineages using cell-specific or lineage-specific promoters, respectively. Accordingly, gene expression using the novel episomal plasmid gene delivery vectors described in this example can be regulated for length of time (constitutive or inducible) and temporally (cell or lineage-type).

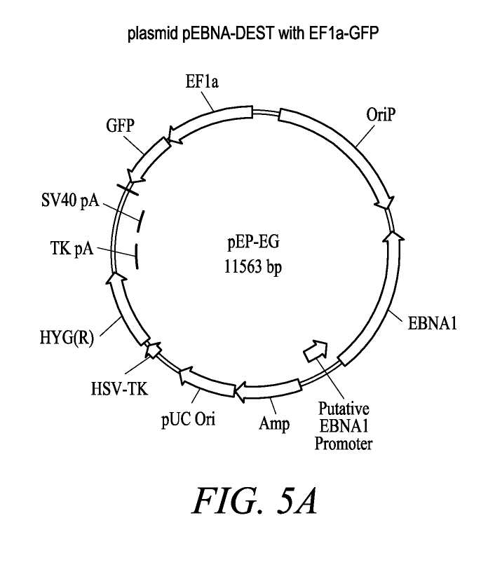

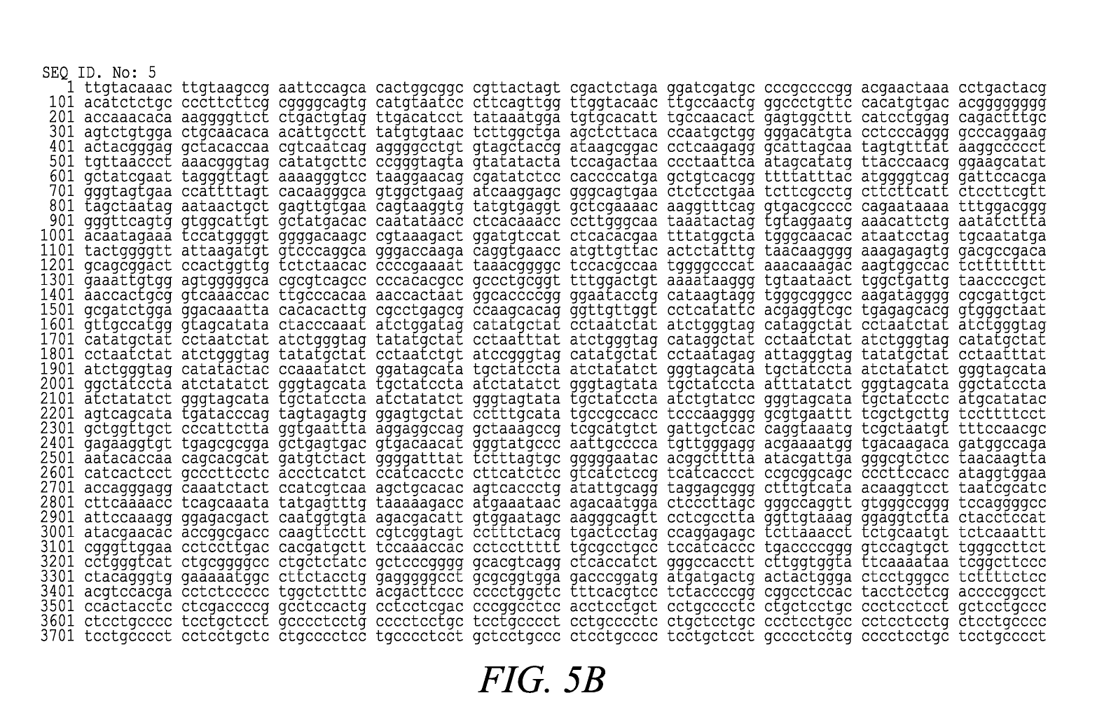

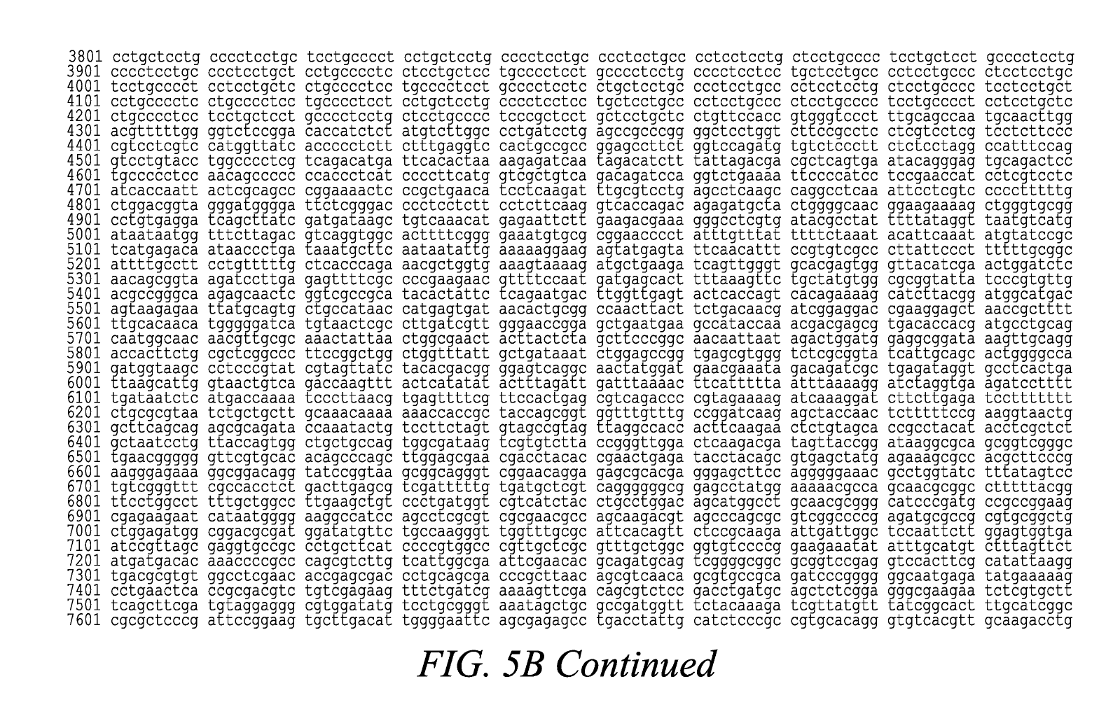

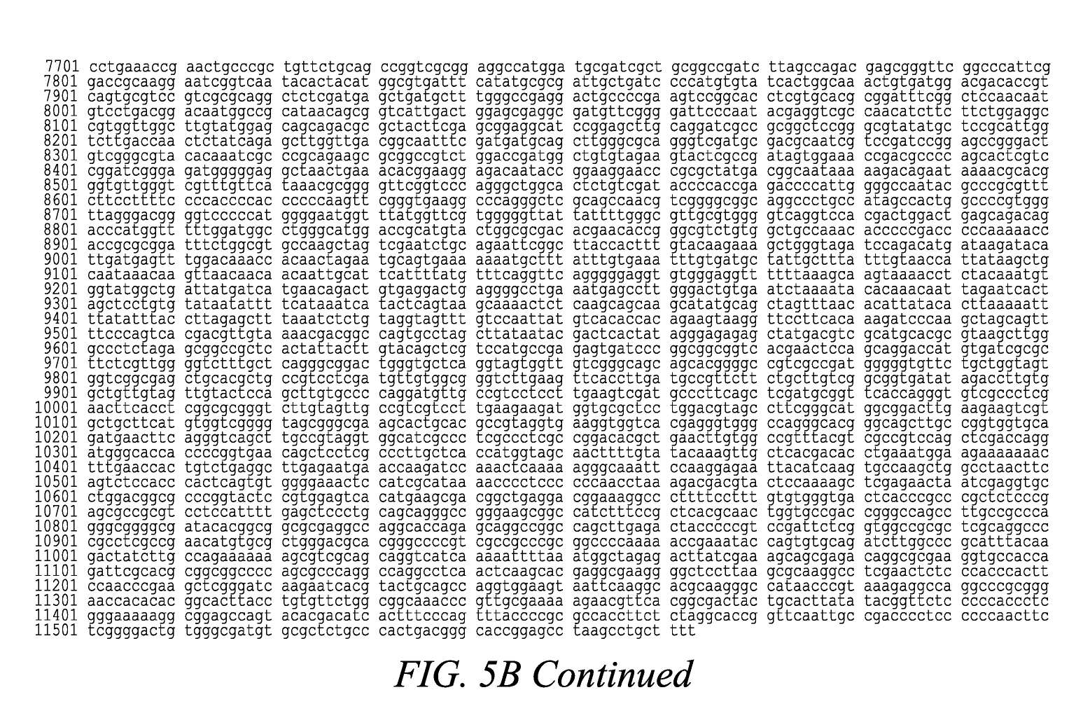

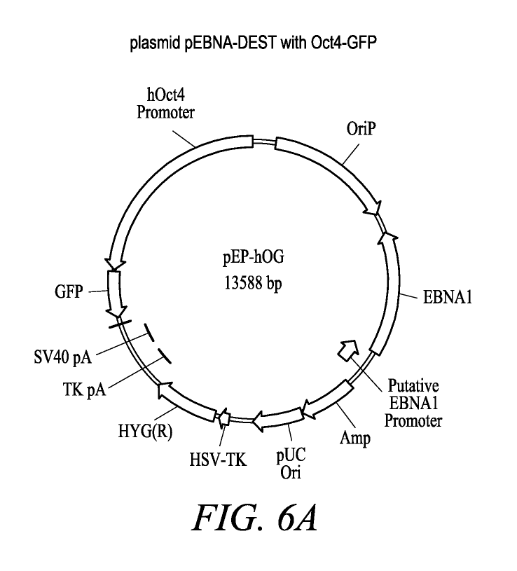

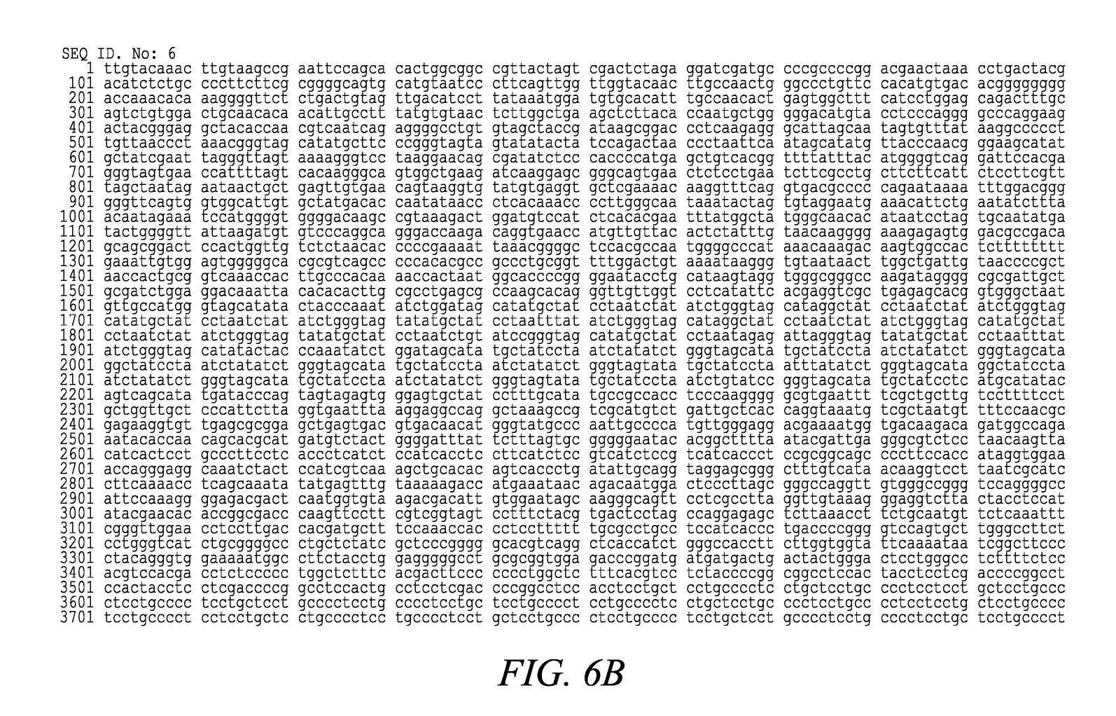

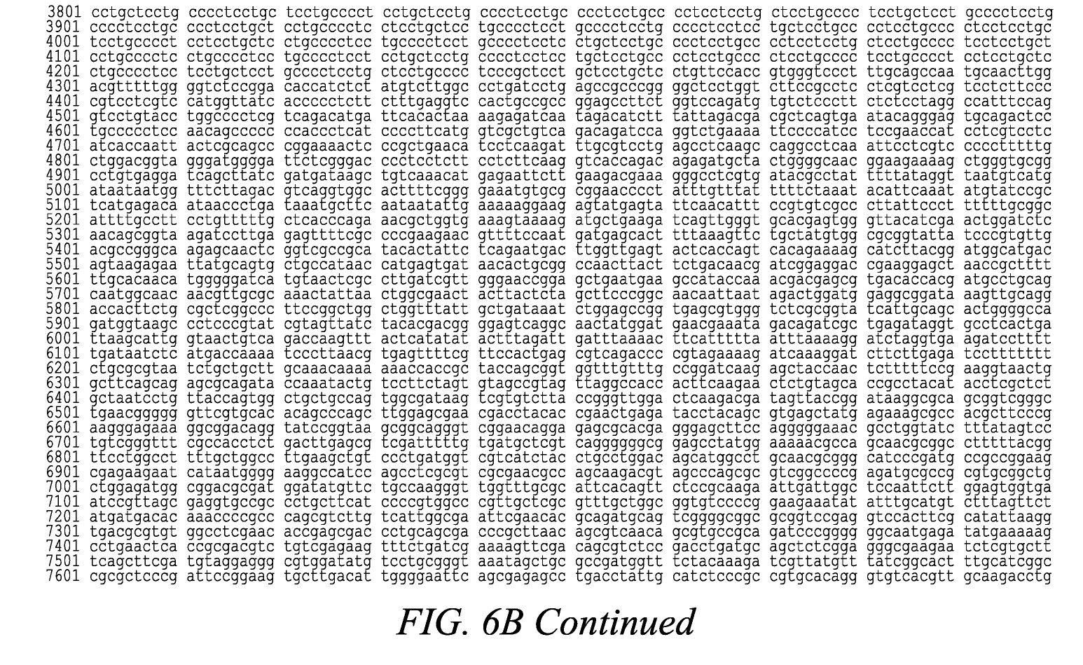

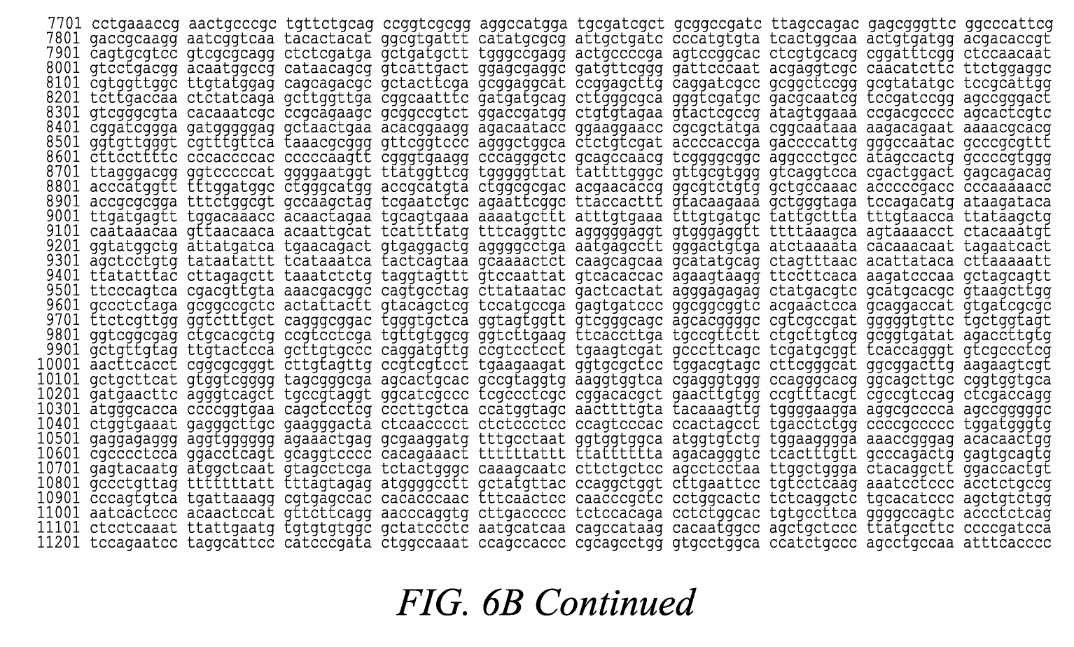

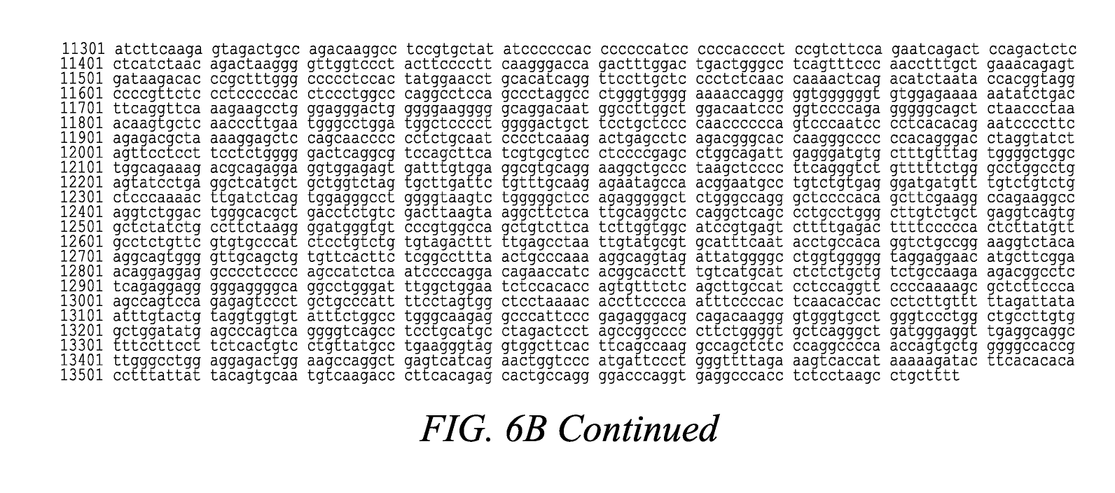

Using the methods described in Thyagarajan et al., (2009), a novel episomal gene delivery vector system, pEBNA-DEST FIG. 4 (SEQ ID NO.: 4), with MultiSite Gateway® assembly. Exemplary episomal expression vectors are also described, for example, the expression plasmid with the EF1a promoter-GFP expression cassette [FIG. 5; SEQ ID NO.: 5] or the Oct4 promoter-GFP expression cassette [FIG. 6; SEQ ID NO.: 6], both of which were are maintained episomally in human embryonic stem cells (hESC). A variant hESC line, BG01V was transfected, using the Microporator, with the vectors described in SEQ ID NO.: 5 and 6. The hESC cell lines thus derived were pEPEG-BG01V, which constitutively expresses GFP under EFla promotion, and pEPOG-BG01V, where the Oct 3/4 promoter drives GFP expression. GFP positive cells were analyzed by FACS analysis and by fluorescence microscopy. These vectors also expressed a drug-resistance marker that allowed for selection and long-term maintenance of cells harboring this vector. In a study for stability of expression of the episomal vector in hESC, they were found maintained in hESC for over 4 months in culture and sustained freeze/thaw.

Sustained expression of GFP in undifferentiated hESC and in their differentiating embryoid bodies was detected. Cultures showed ˜50 to 96.41% GFP positive cells even after 4 weeks (between passages 8 to 12) even without antibiotic (hygromycin) selection.

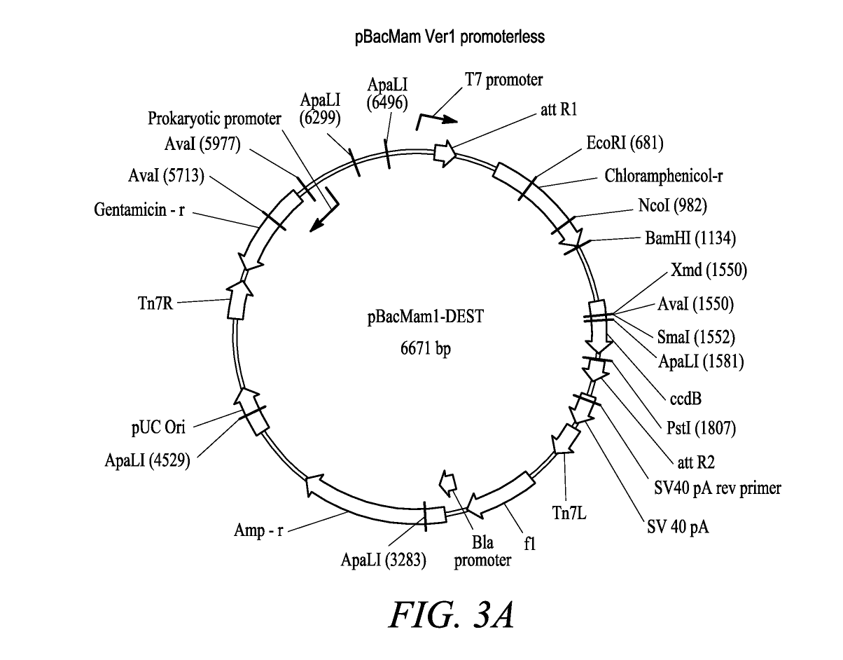

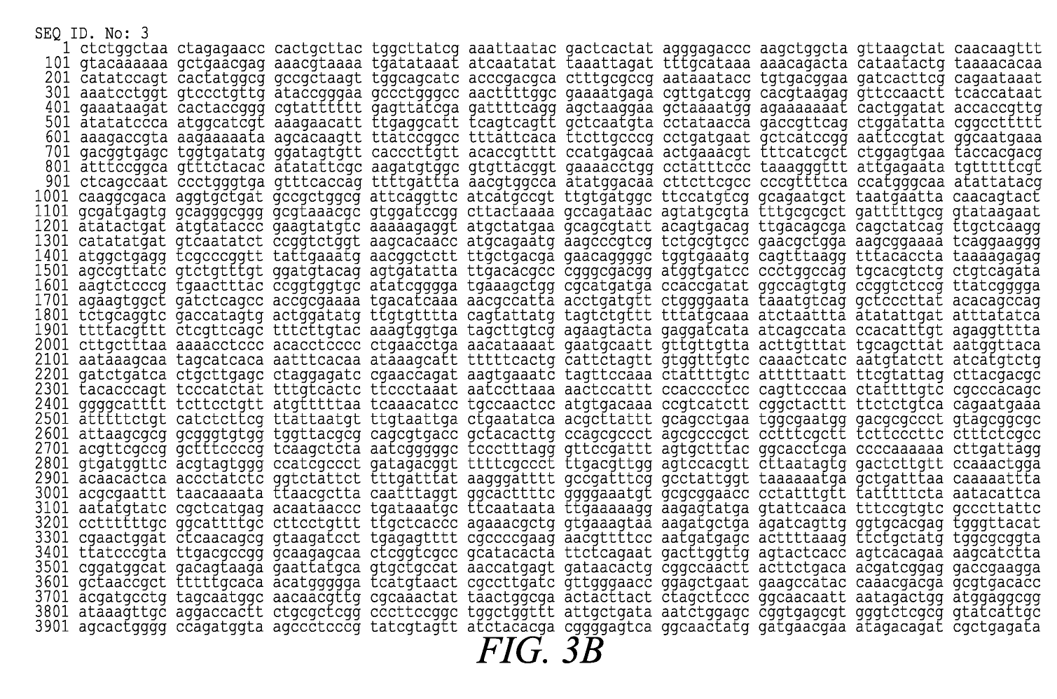

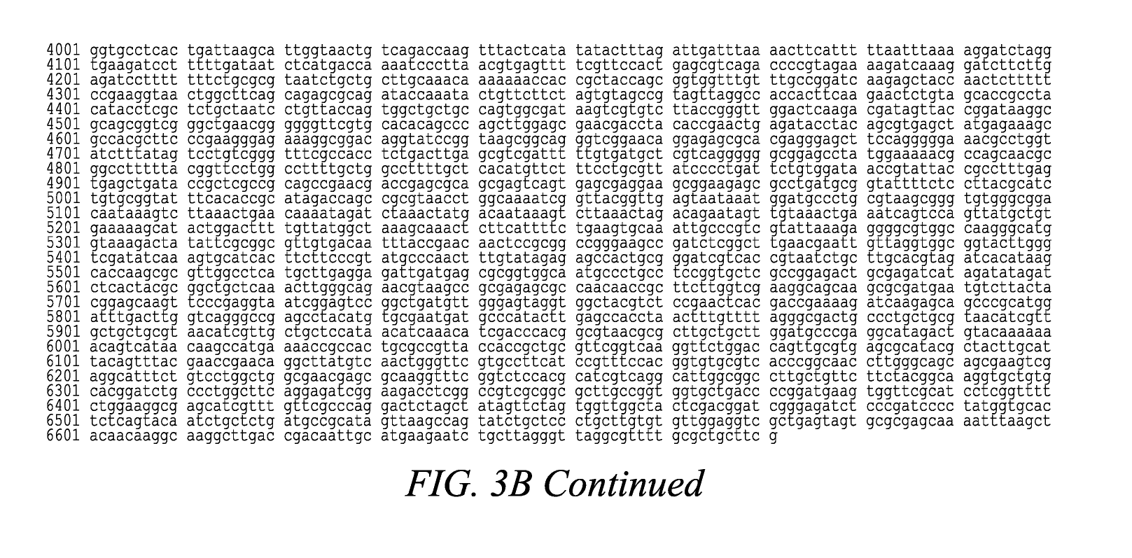

These hESC cell lines were also studied for stability of GFP expression during the process of differentiation. pEPEG-BG01V cells were differentiated using standard differentiation protocol and then, expression of GFP was analyzed using analysis and by fluorescence microscopy. Stable episomal clones continued to express pluripotent markers and differentiation markers. Episomal expression of GFP was seen in bulk adipose tissue-derived mesenchymal stem cells through differentiation of the hESCs into adipocytes, osteoblasts and chondroblasts. This showed that gene expression from the episomal vector was stable during differentiation of the cell. Furthermore, the stable hESC clones showed comparable expression with and without drug selection (see FIGS. 3A to 3C of Thyagarajan et al., Regenerative Medicine (2009)). Therefore, these single episomal vectors offer an easy and rapid method to modify stem cells, to generate either stable pools or as heterogeneous bulk cells, which can then be used for various downstream applications. The product literature for the pEBNA-DEST vector kit [Invitrogen Cat. No. A10898], which describes how one skilled in the art can construct multiple-fragment expression vectors, disclosure of which is hereby incorporated by reference in its entirety. Cloning of any genetic element of interest can be accomplished using the MultiSite Gateway® Technology in the pEBNA-DEST vector, which allows for rapid and efficient cloning of multiple genetic elements of interest (such as promoter-reporter pairs) in a defined order and orientation.

### Example 2

### MultiSite Gateway® Episomal BacMam Viral Vectors: Constitutive and Inducible Viral Gene Delivery Systems

Novel gene delivery viral vectors were developed that do not stably integrate into the cell's genome, but instead, are either (i) maintained stably episomally due to constitutive expression of the EBNA1 gene, and thereby stably sustaining reprogramming gene expression during the period of reprogramming; or (ii) can be induced to sustain reprogramming gene expression during the period of reprogramming due to inducible expression of the EBNA1 gene, and later, can be turned off once cells have been reprogrammed, or the desirable level of reprogramming has been achieved. These gene delivery viral vectors can introduce one or more reprogramming genes at a given time into a given mammalian cell. Viral vector systems generally use an insect virus as a gene delivery system (for example, baculovirus); in this invention BacMam Ver 1 and BacMam Ver 2 family of vectors described in Table 1 were used. The vectors carry one or more genes, or a set of reprogramming genes, into mammalian cells. The backbone of the baculovirus is used to generate BacMam viral vectors. The Ver 2 family of BacMam vectors described in Table 1, namely [SEQ ID NOs: 8, 9, 10, 11, 12] additionally comprise the WPRE (WoodChuck Hepatitis Posttranscriptional Regulatory Element) and the VSV-G expression cassette (Vesicular Stomatitis Virus G protein), which mediates viral entry into a variety of mammalian cells. The viral vectors of the invention are defined herein in Table 1.

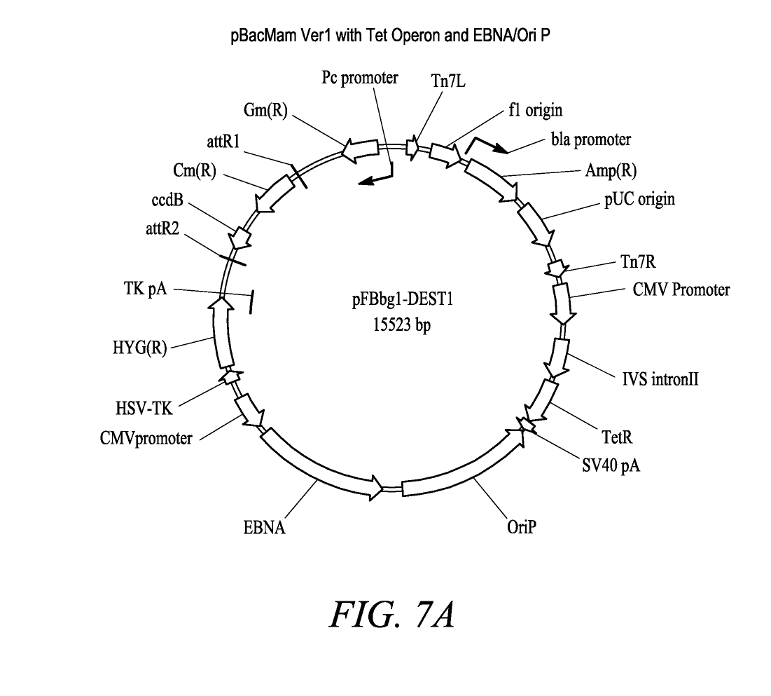

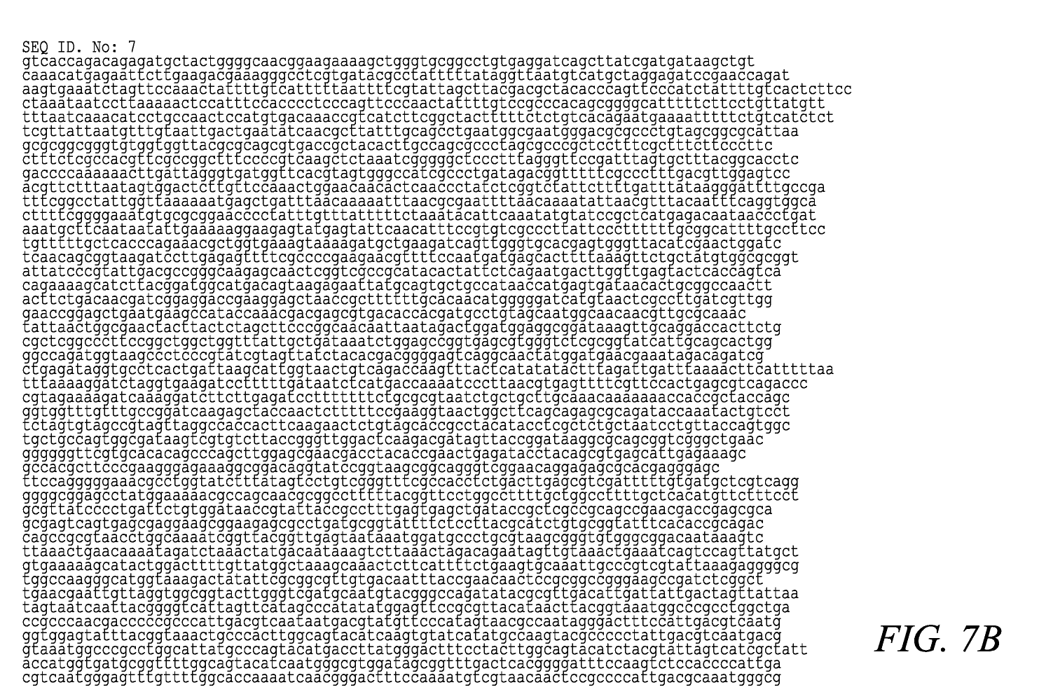

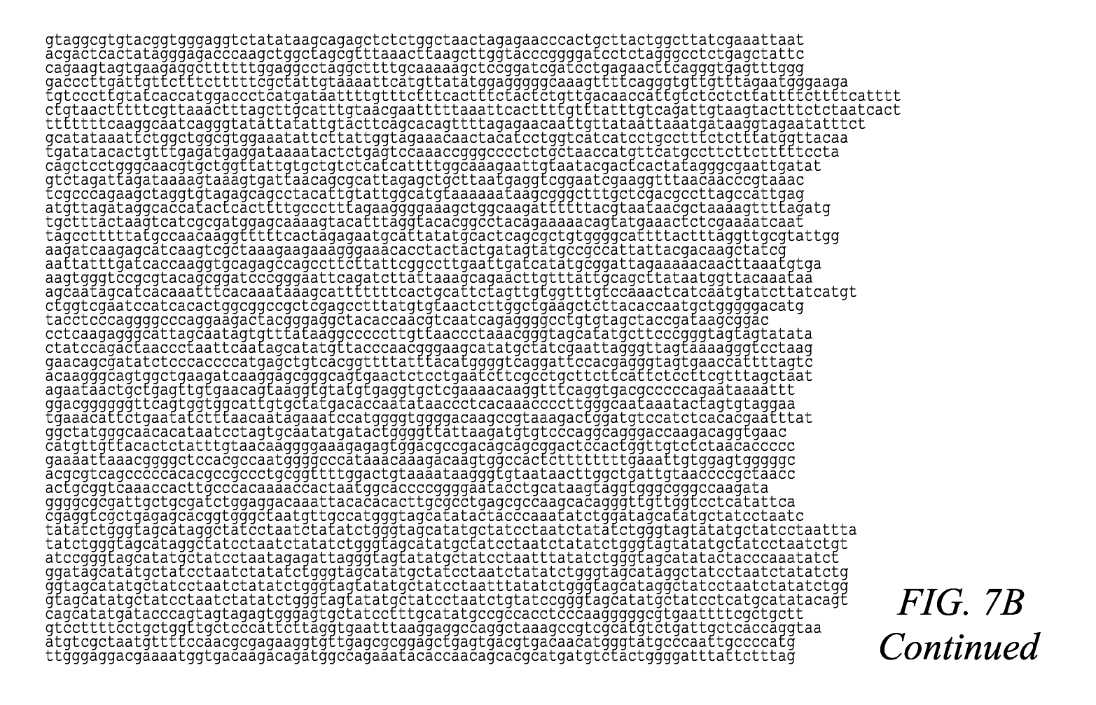

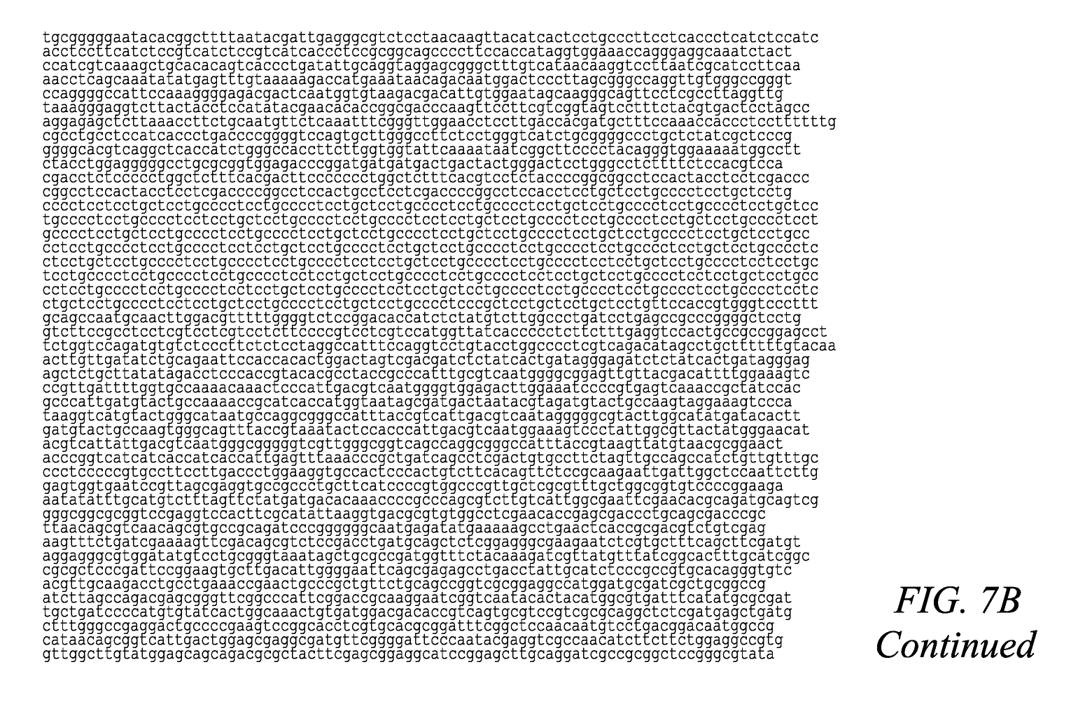

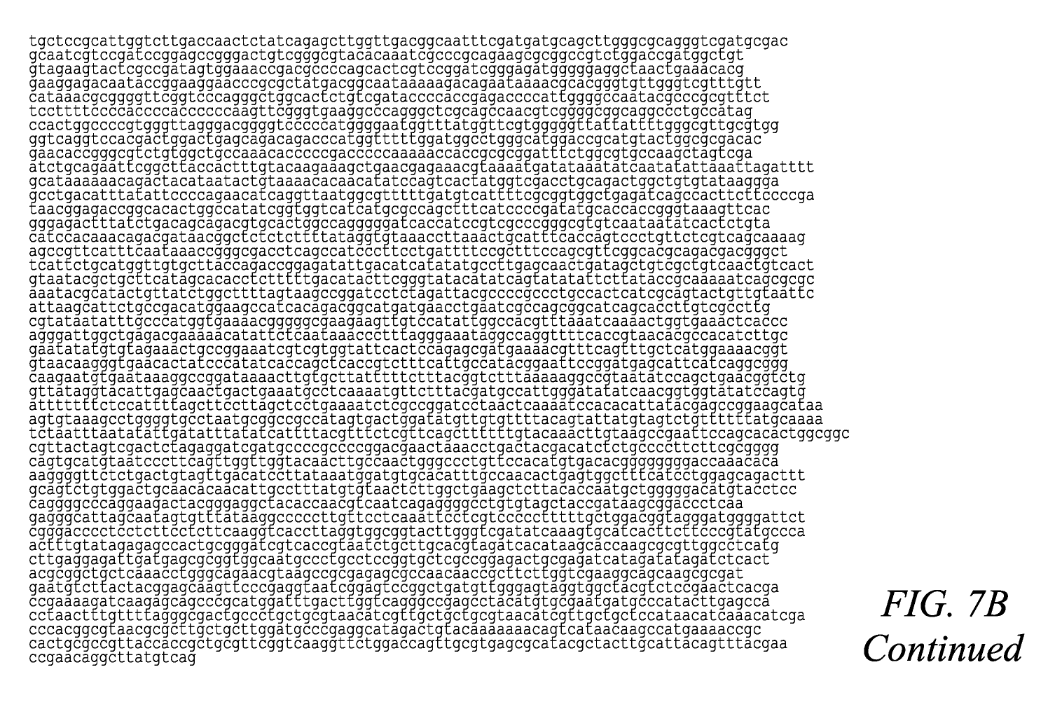

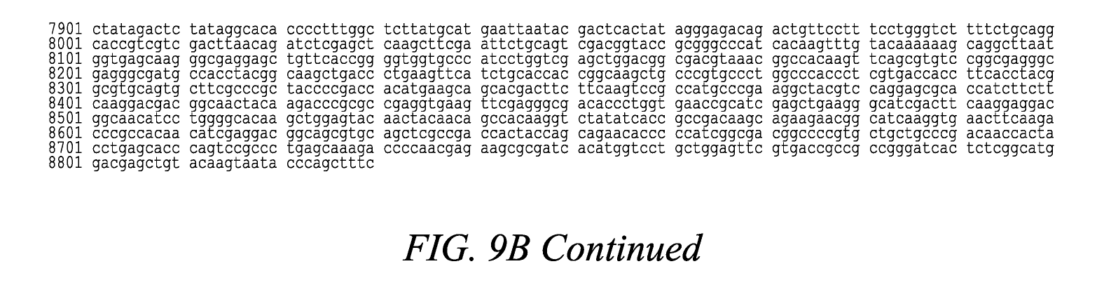

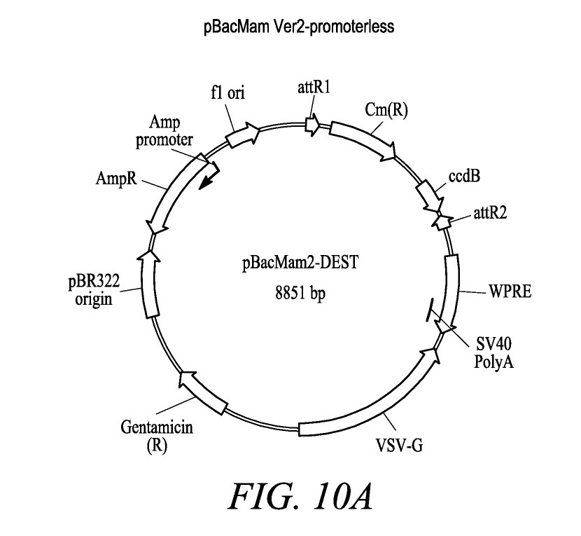

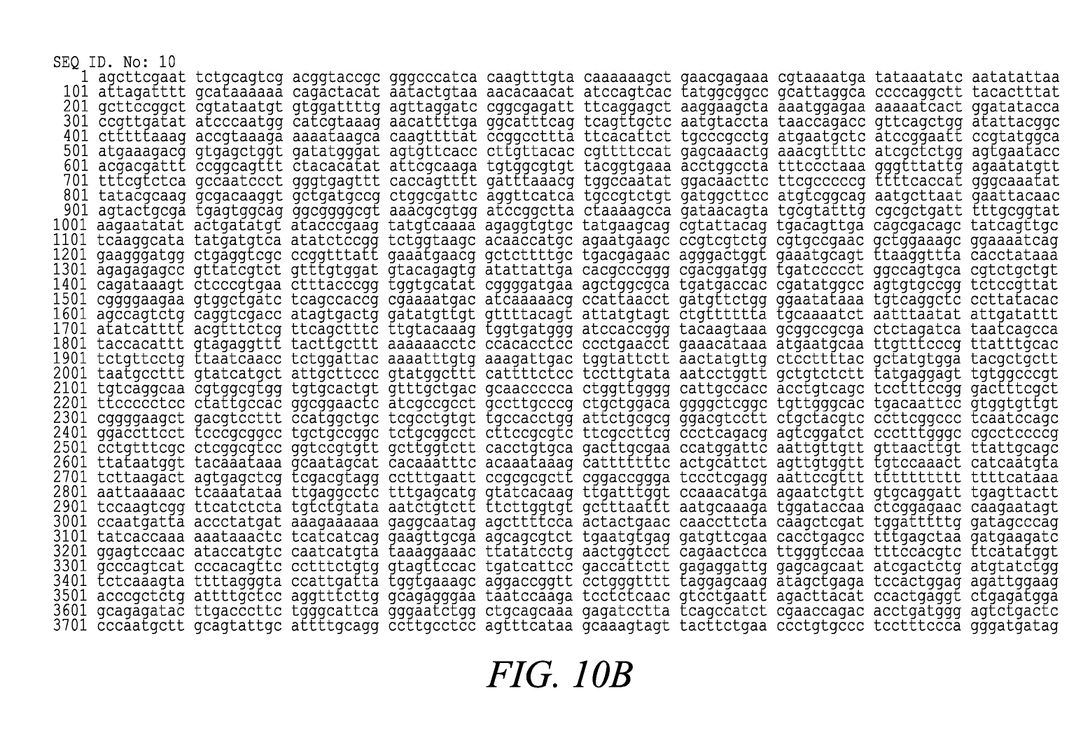

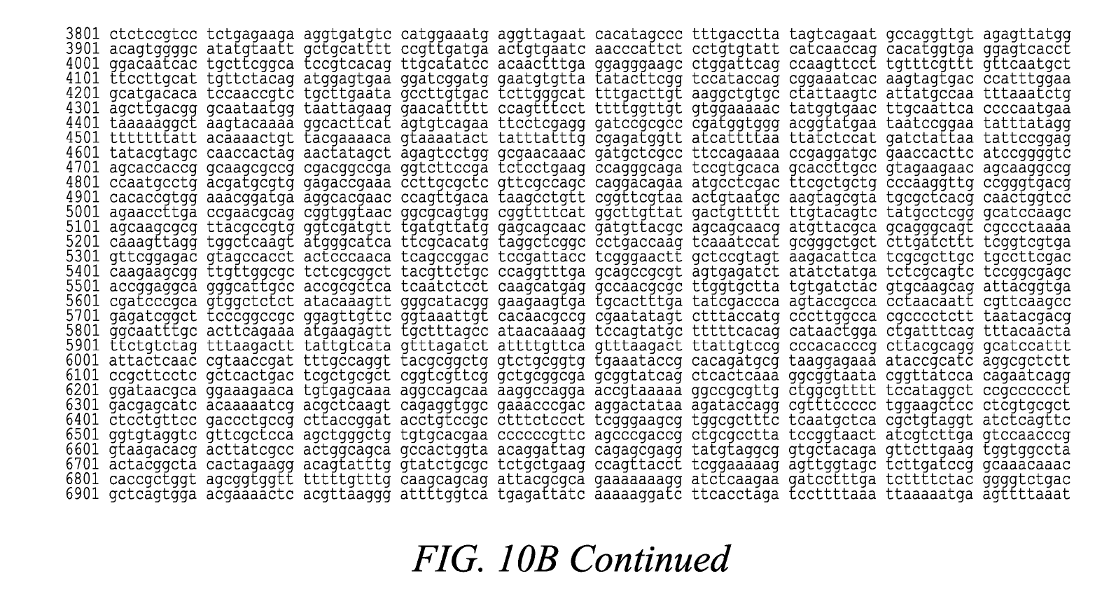

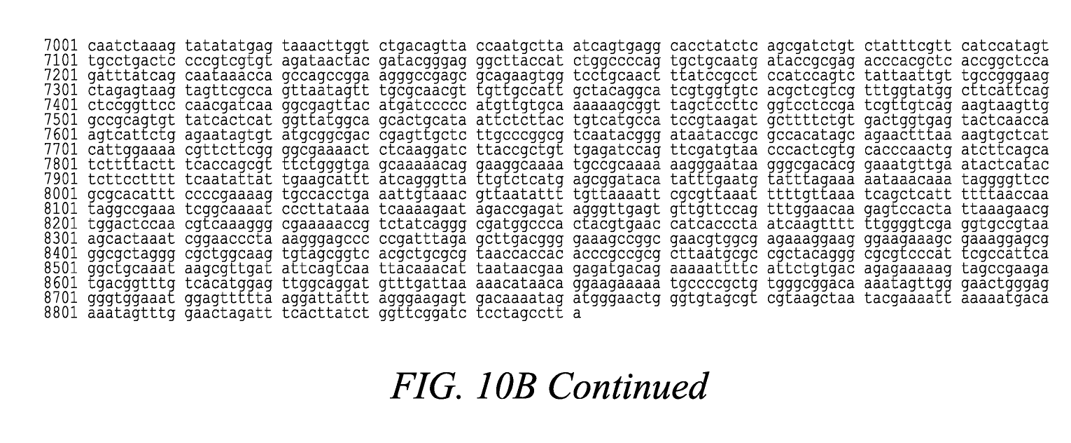

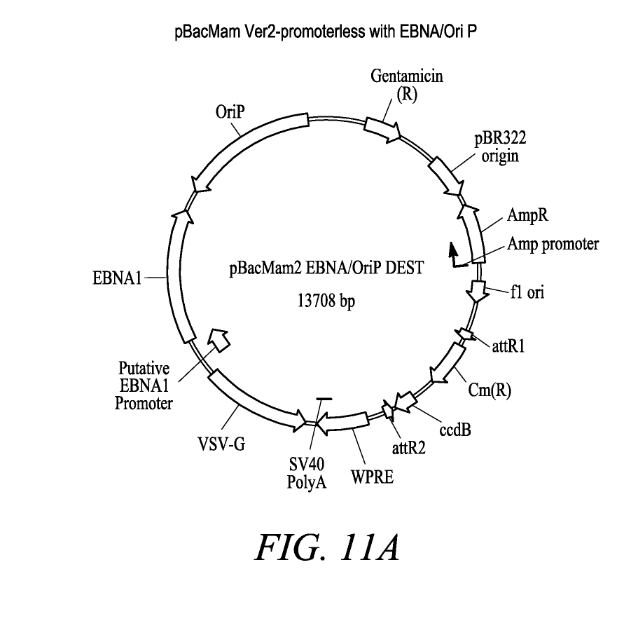

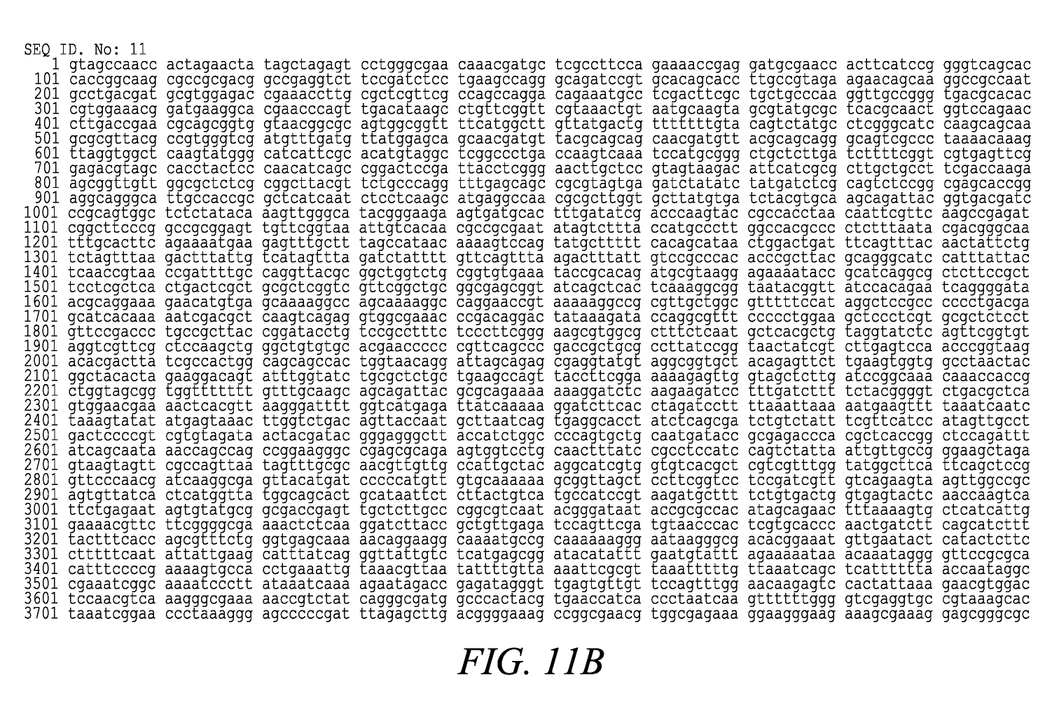

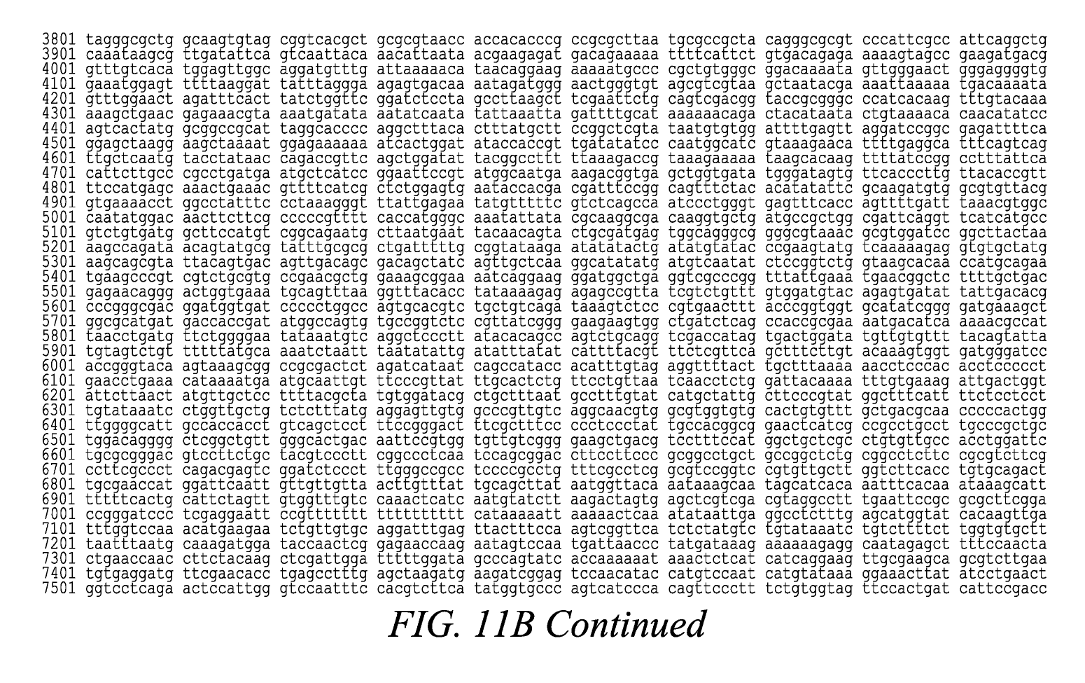

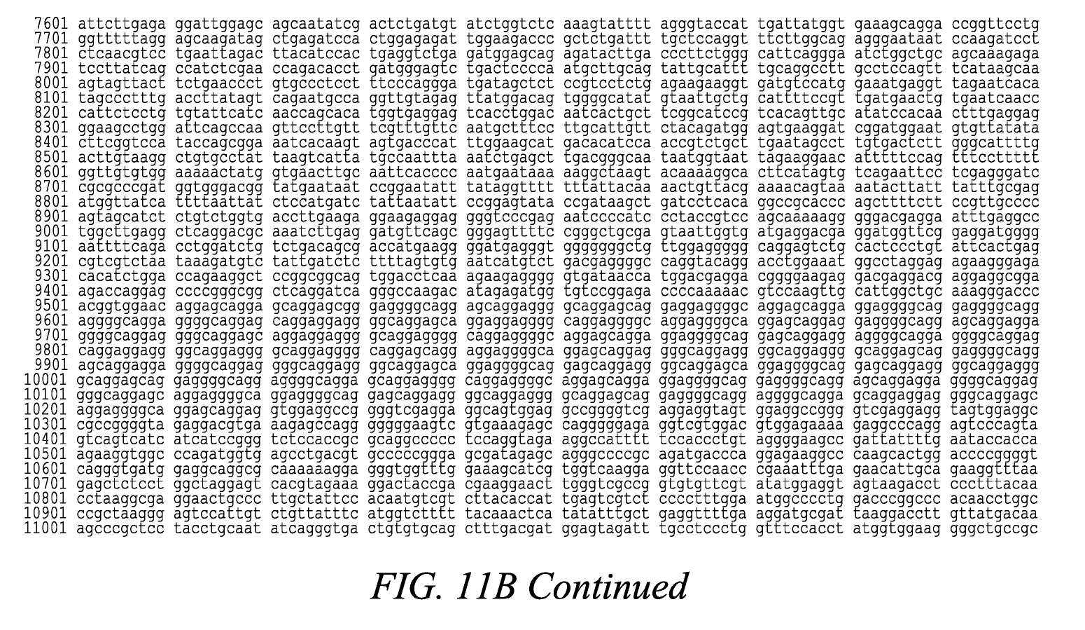

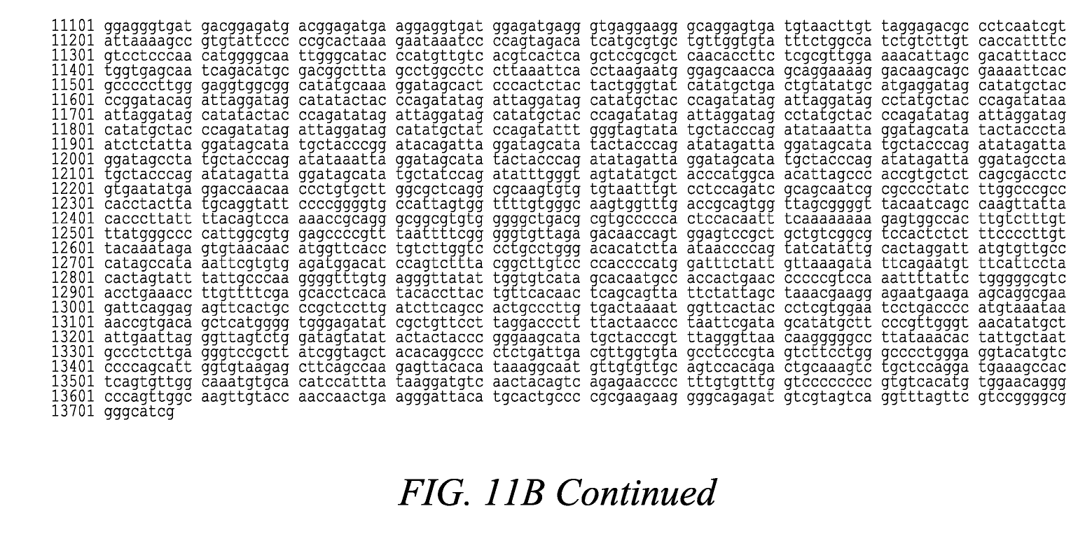

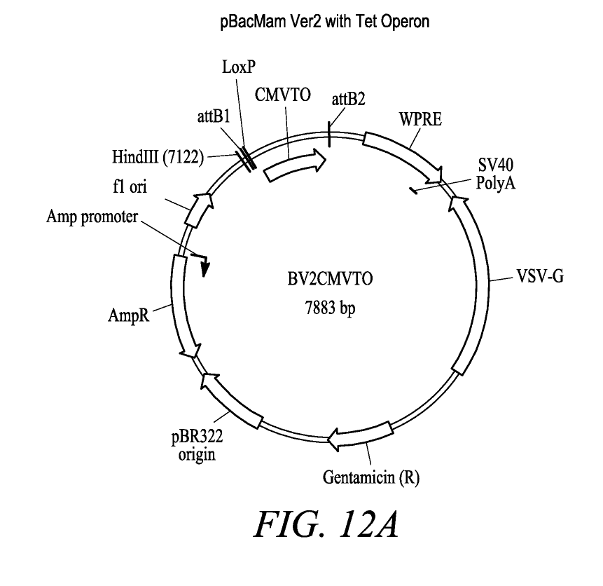

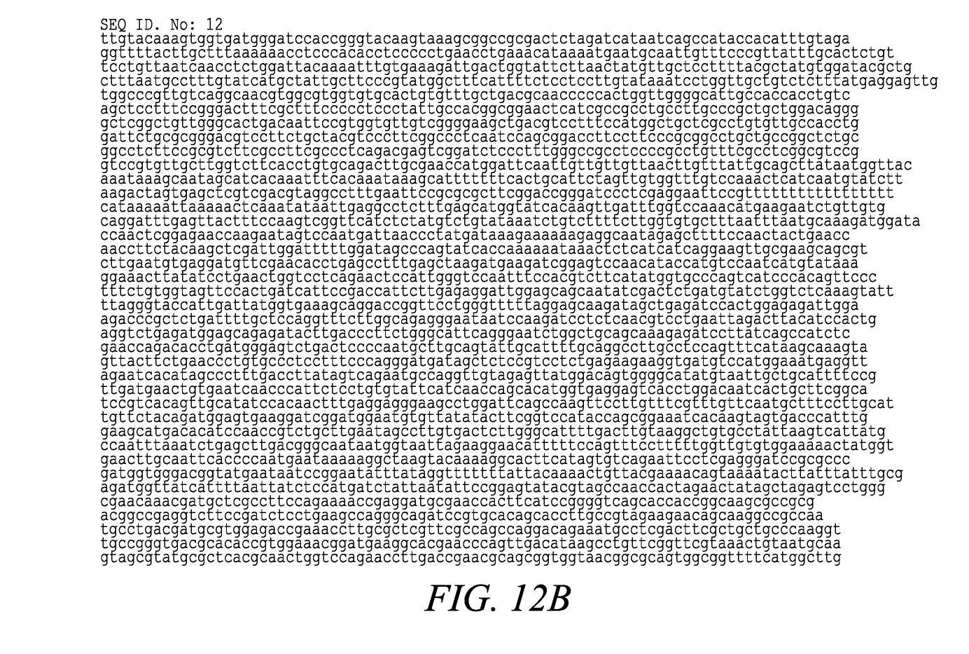

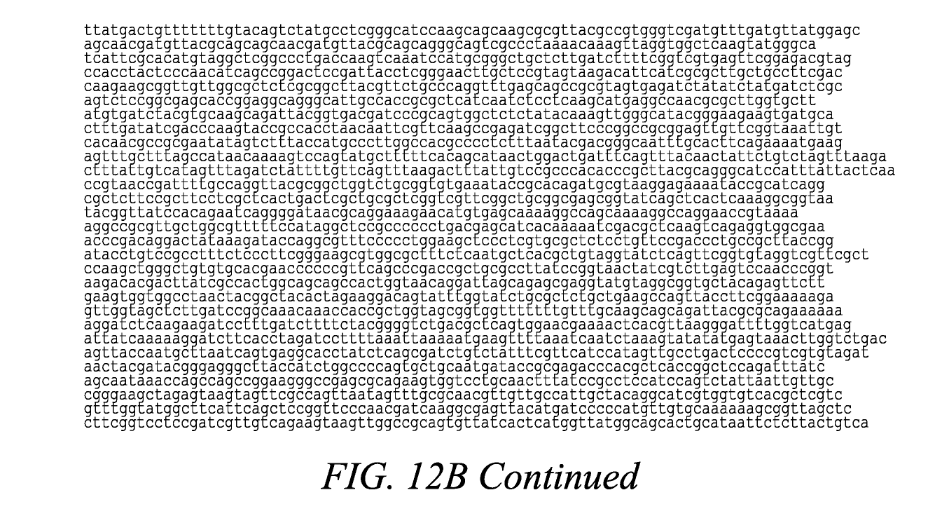

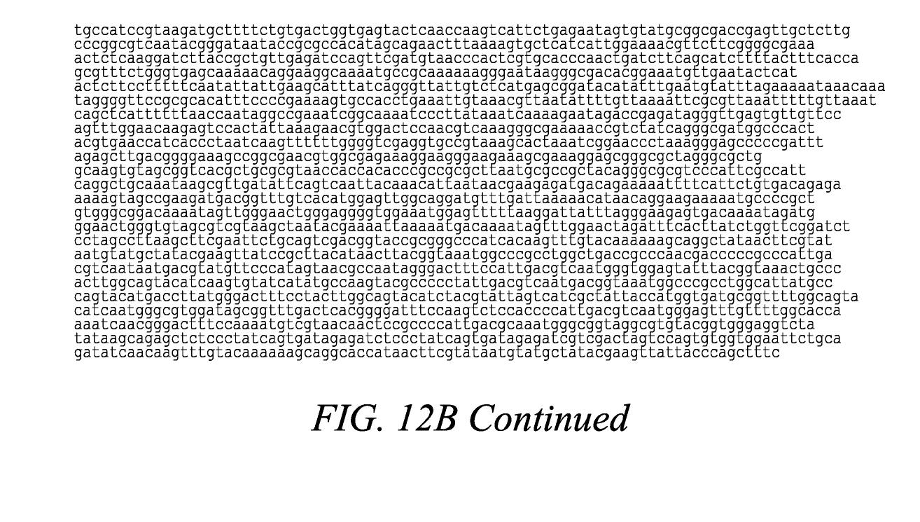

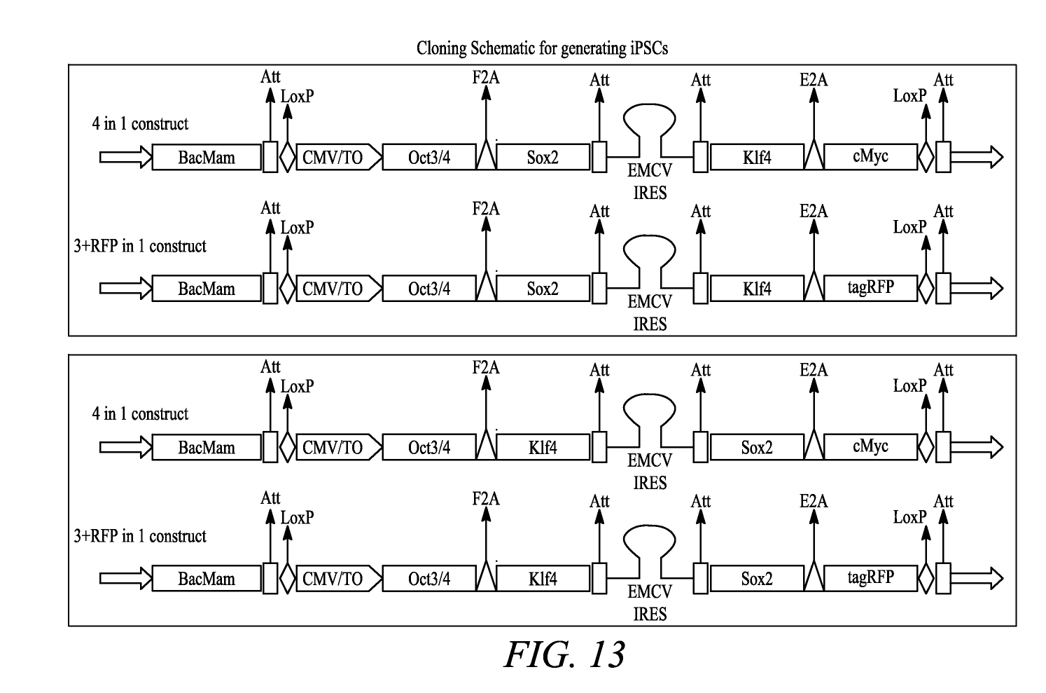

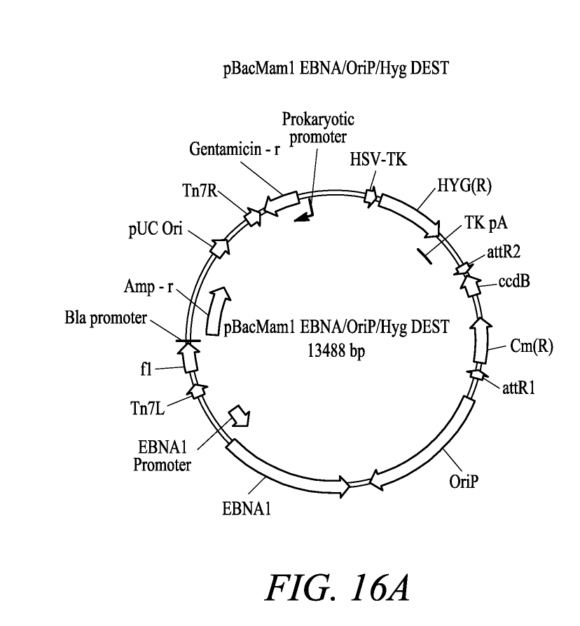

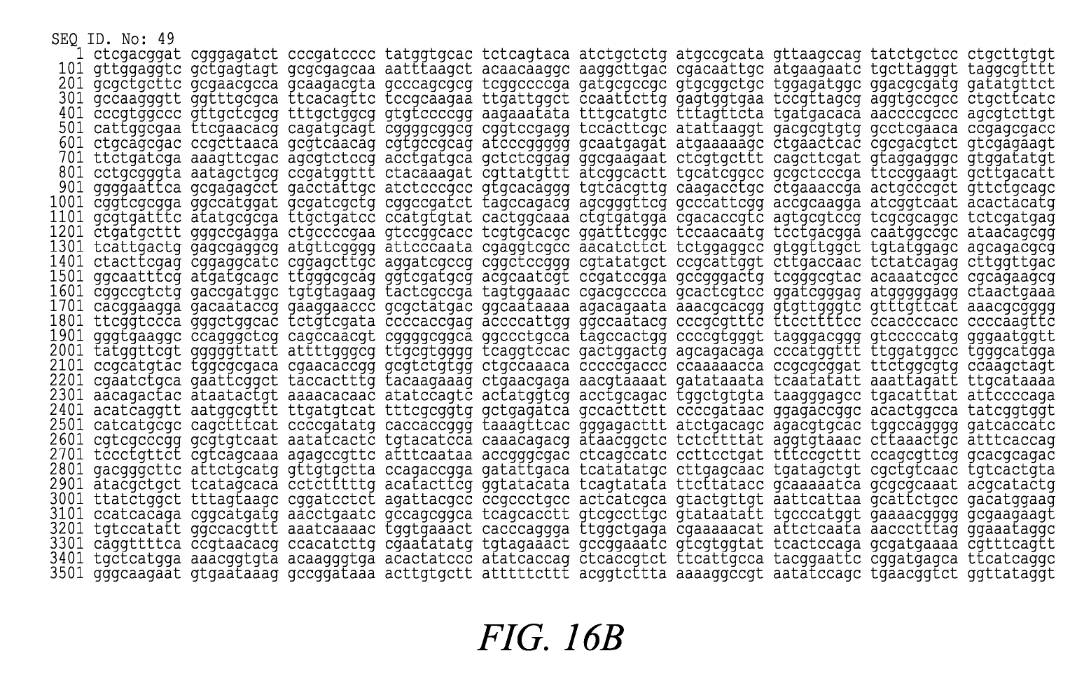

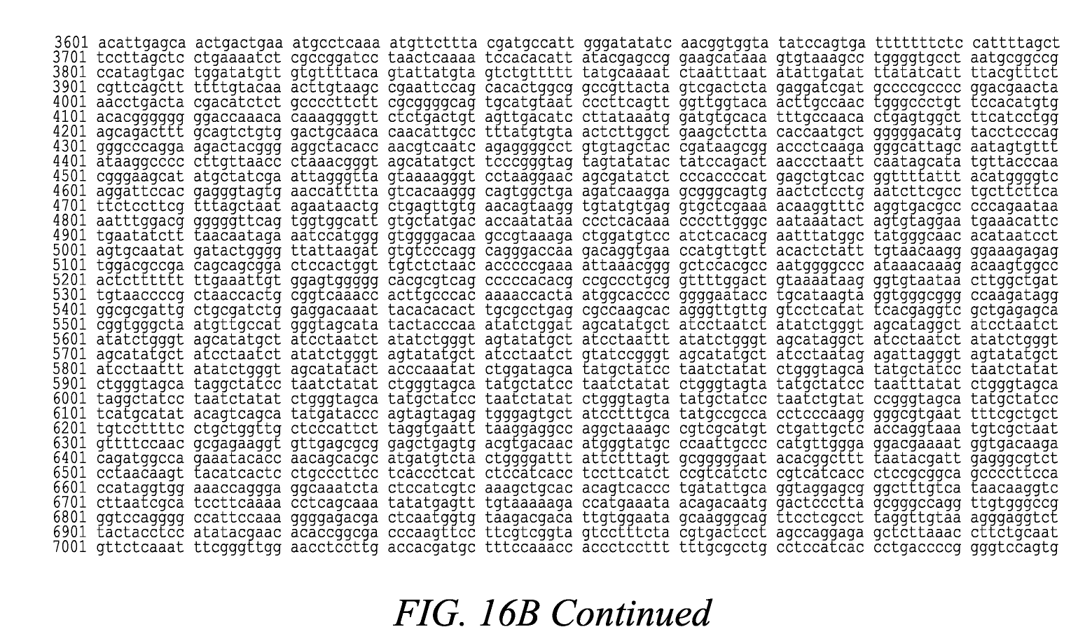

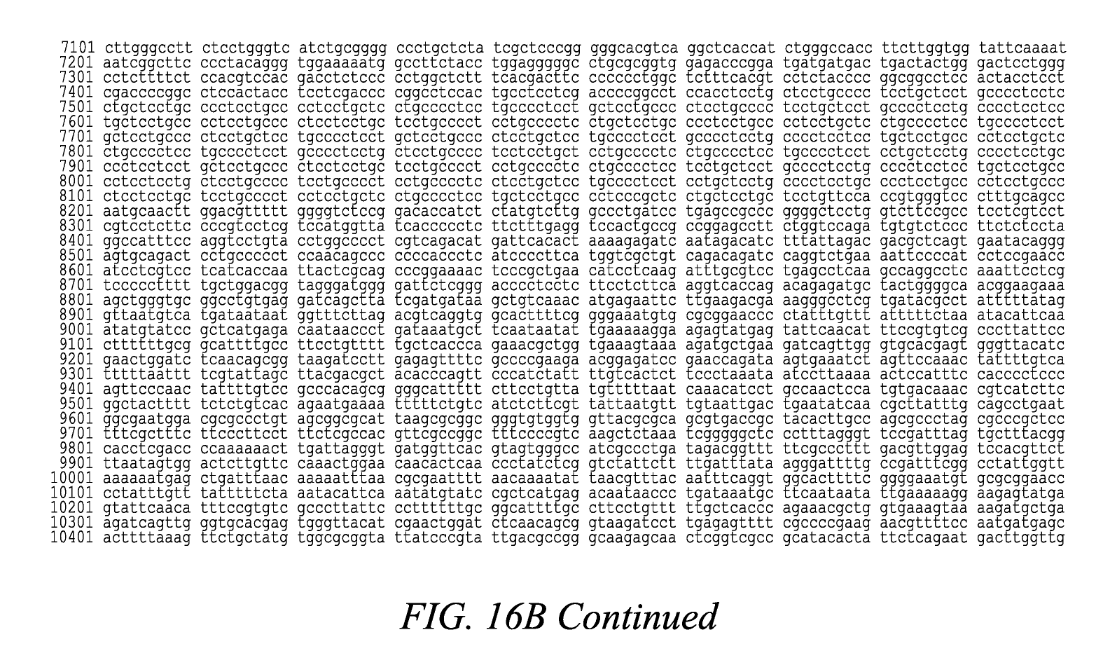

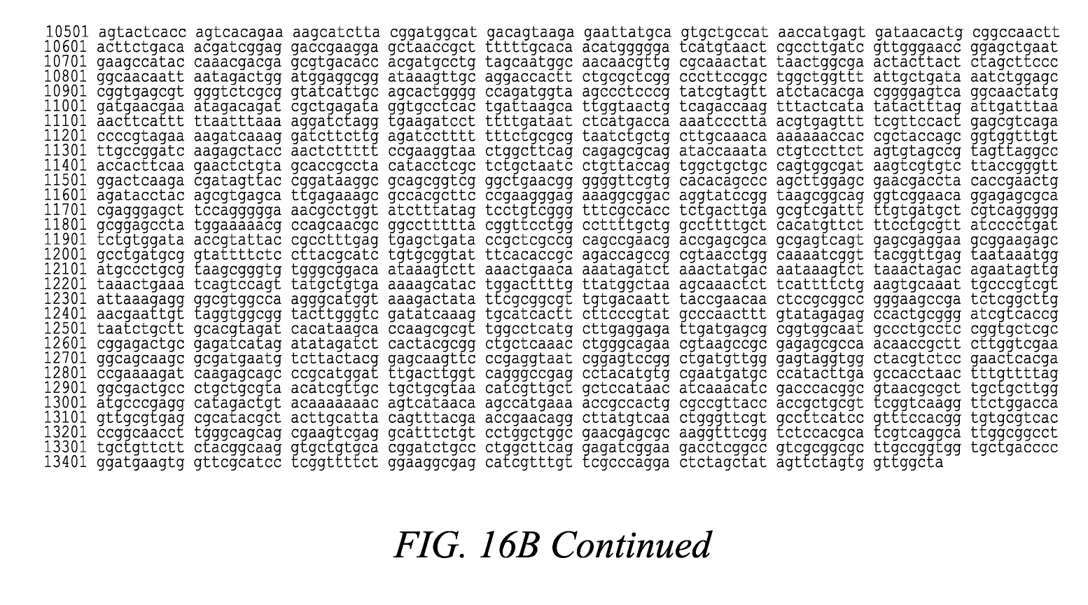

TABLE 1
Viral (BacMam) Vectors for Gene Delivery

| | | | | Expression | |
|---|---|---|---|---|---|
| FIG. No.; | | | | Cassette | Vector |
| SEQ. No. | Name | Other names | Description | Promoter | Type |
| FIG. 2; | pBacMam ™ | pBacMam1 | Original Gateway | CMV | DEST |
| SEQ ID NO. 2 | Ver1 DEST | DEST CMV; | adapted BacMam | | vector |
| | CMV | pDEST8-CMV | Ver 1 | | |
| FIG. 8; | pBacMam ™ | pBacMam2 | BacMam Ver 2 | CMV | DEST |
| SEQ ID NO. 8 | Ver2 DEST | DEST CMV; | from MGH | | vector |
| | CMV | pHTBV1.1 | | | |
| FIG. 3; | pBacMam ™ | pBacMam1 | promoterless | None | DEST |
| SEQ ID NO. 3 | Ver1 DEST | DEST; | pBacMam Ver1 | | vector |
| | | pDEST8 | DEST | | |
| FIG. 10; | pBacMam ™ | pBacMam2 | promoterless | None | DEST |
| SEQ ID NO. | Ver2-DEST | promoterless | pBacMamVer2 | | vector |
| 10 | | | DEST | | |
| FIG. 7; | pBacMam ™ | pFBbg1- | pBacMam Ver 1 | None | DEST |
| SEQ ID NO. 7 | Ver1/TO/ | DEST1; | with Tet Operon | | vector |
| | EBNA/OriP | pDEST8- | driven EBNA | | |
| | DEST | Hygro-EBNA | | | |
| FIG. 16; | pBacMam ™ | pBacMam1 | pBacMam Ver 1 | None | DEST |
| SEQ ID NO. | Ver1 | EBNA/OriP/ | with Constitutive | | vector |
| 49 | EBNA/OriP | Hyg DEST | EBNA | | |
| | DEST | | | | |
| FIG. 11; | pBacMam ™ | pBacMam2 | pBacMamVer2 | None | DEST |
| SEQ ID NO. | Ver2 | EBNA/OriP | with Constitutive | | vector |
| 11 | EBNA/OriP | DEST; pEP- | | | |
| | DEST | BV2-DEST | EBNA | | |
| FIG. 12; | pBacMam ™ | pEP-BV2- | pBacMam ™ 2 | None | DEST |
| SEQ ID NO. | Ver2 | DEST | with Tet Operon | | vector |
| 12 | EBNA/OriP | | driven EBNA | | |
| | DEST | | | | |

The BacMam episomal vectors of the present invention also expressed the EBNA1 gene/OriP elements. Transduction of BacMam-EBNA1 episomal vectors where EBNA1 expression is under a constitutive promoter, into a mammalian cell, results in the stable expression of the EBNA1 protein that binds to the OriP to facilitate the retention and replication of the OriP containing vectors, ensuring its expression during the reprogramming period. Therefore expression of reprogramming genes in the expression cassette of the vector will result in stable expression of reprogramming genes. This may be desirable in certain systems where sustained expression of reprogramming genes is necessary to maintain a reprogrammed phenotype. On the other hand, transduction of episomal viral vectors that inducibly express the EBNA1 protein (due to say an inducible promoter like the Tet operon), and growth of the cell in the presence of tetracycline will result in the transient expression of the EBNA1 protein, ensuring its expression only in the presence of tetracycline (which can be regulated for the desired reprogramming period). Once reprogrammed cells or induced pluripotent cells (iPCs) are obtained, the tetracycline is removed resulting in the repression of the EBNA1 protein by the tet repressor, and the episomal viral vector will not be maintained. After a couple of rounds of cell division, the viral vector is lost (since this delivery system does not integrate into the cell's genome) and no footprint of the viral vector is left. This may be desirable in some applications, for e.g., for therapeutic purposes with no viral vector remanants.

Inducible viral episomal vectors defined by SEQ ID NOs: 7 and 12 comprise DNA segments that express the Tet repressor, an inducible CMV/TetOperon promoter driving the EBNA1 gene, a cis OriP (for the maintenance of the vector during cell division), and a hygromycin selectable marker, and further, components that express the MultiSite Gateway® cloning cassette to enable cloning of multiple reprogrammable genes.

A single baculoviral vector, pFBbg1-DEST1, as exemplified in FIG. 7, was generated to further reduce the total number of viral vectors required for transduction. Primary fibroblasts were transduced with the novel vector compositions described above, and screened with antibodies to stem cell marker genes such as Oct3/4, Nanog, SSEA1, and TRA1-80. iPS cells so identified were propagated and allowed to form embryoid bodies to allow spontaneous differentiation into three primary germ layers. Differentiated germ layers were stained with markers for neurons (e.g., bIII tubulin, Nestin), mesoderm (SMA, smooth muscle actin), and endoderm (alpha fetal protein).

Subcutaneous injection of the iPS cells (generated by the methods of the invention) into severe combined immunodeficiency (SCID) mice were tested for teratoma formation, and iPS cells capable of undergoing a stable transition to the pluripotent state were also identified.

The second part of this study was to identify molecules that enhance reprogramming efficiency. Since overall, the efficiency of reprogramming is between 0.1%-5%, screening was done for DNA methyltransferase inhibitors, a set of miRNAs that are highly differentially expressed in hESCs. Factors involved in the maintenance of pluripotency were screened for their ability to enhance reprogramming efficiency. In addition, the reprogrammed cells were also cultured in serum free medium containing enhancing molecules identified in this study, to generate iPS cell lines suitable for clinical studies.

Constitutive viral vector systems based on components derived from the Epstein-Ban virus were maintained episomally in primate and canine cells over long periods of culture. This vector system was adapted to Multisite Gateway cloning, which enables rapid assembly of expression constructs that can be used to engineer human embryonic stem cells (ESC) and mesenchymal stem cells (MSC). To demonstrate the utility of this system, we created ESC pools with vectors containing GFP driven by either a constitutive promoter (EF1a), an embryonic stem cell-specific promoter (Oct4) or a hepatocyte-specific promoter (AFP). When GFP expression was driven by a constitutive EF1a promoter, expression was seen in undifferentiated as well as differentiated cells. When the Oct4 promoter was used, GFP was expressed only in undifferentiated cells. AFP-GFP containing cells showed GFP expression only in a small subset of differentiated cells that also stained positive for AFP. We have used this vector system to successfully engineer ESC with vectors containing multiple reporters and as large as 20 kb. We have shown that these vector systems are also functional in other stem cells. MSC transfected with episomal vectors showed sustained expression for over 3 weeks in the absence of drug selection. These MSC differentiated into adipocytes, osteoblasts and chondroblasts, and continued to express GFP throughout the process of differentiation.

Methods for tracking cell types of interest during the process of differentiation. The constitutive BacMam vector, e.g., pEP-FB-DEST1 FIG. 16, SEQ ID NO: 49 may be used for the expression of any fluorescence protein or selectable marker described in the invention. Regulation may, for example, be under the native EBNA1 promoter, any constitutive promoter known in the art, or a lineage-specific or tissue-specific promoter. A constitutive promoter may be a strong viral promoter like the CMV promoter.

Reprogramming Cells Using Transient and Constitutive BacMam Particles

Using a platform based on BacMam, a baculovirus-mediated gene delivery method that efficiently transduces hard-to-transfect cells was generated. We modified the BacMam vectors, version 1 and 2 (SEQ ID NOS: 2 and 8) with EBNA1 and OriP, to generate viral vectors (SEQ ID NOS: 49 and 11) to allow for long-term maintenance of these vectors in transduced cells. The BacMam vectors Ver 1 (SEQ ID NO: 2) and Ver 2 (SEQ ID NO: 8) were further modified to create a promoterless version to enable cloning any promoter/reporter gene combination of choice (SEQ ID NOs: 3 and 10 respectively). This further enables use of lineage-specific or cell-specific promoter/genes of choice. We show that BacMam can consistently transduce mesenchymal stem cells and neural stem cells at over 80% efficiency and can be used to generate uniformly labeled cells.

Further, BacMam vectors were also used to uniformly label adipose-derived mesenchymal stem cells (AdSCs). The expression of the transgene persists for 5-7 days in dividing cells and in day 7 differentiated adipocytes. Using this method, C-Jun, a key pathway in differentiating adipocytes was validated in ADSCs utilizing the Lanthascreen® time-resolved FRET based assay. To extend the expression length to longer periods of time and to enable delivery into additional primary and adult stem cells types, a vector system with VSV-g and WPRE was utilized. Using this enhanced BacMam, both mesenchymal stem cells and neural stem cells were transduced at over 80% efficiency. Persistenace in expression lasted for over 10 days with minimal attenuation of GFP signal intensity both in dividing AdSC and differentiated adipocytes. The Multisite Gateway adapted vectors in this platform enable assembly of lineage specific reporters for efficient delivery and transient expression of reporters. To extend the use of BacMam for the creation of stable cells, we have modified the BacMam vector with EBNA1 and OriP. Preliminary data indicates that transgene expression is maintained for over 3 weeks in transduced cells using these hybrid BacMam vectors.

Further, using human AdSC [Adipocyte derived Stem Cells] as the cell model, key pathways were identified using Illumina gene expression pattern in undifferentiated cells and during various stages of adipogenesis which was in turn used to identify active signaling pathways. Given the robust expression of c-jun in AdSC and its clustering with genes involved in cell differentiation, further analysis of this pathway was performed. AdSC were transiently transduced with BacMam GFP-c-jun (1-79) followed by the analysis of TNF or anisomycin inducible GFP-jun (1-79) phosphorylation using Lanthascreen® time-resolved FRET based assay. In addition we showed the inhibition of TNF induced GFP-jun (1-79) phosphorylation by SB60025, a well characterized inhibitor of JNK. In conclusion these results demonstrate that BacMam offered an easy and efficient method for the creation of cell based assays in stem cells. Genetically engineered multipotent mesenchymal stromal cells offers a tool for drug screening, in vivo cell tracking, and gene therapy and in basic cellular characterization studies. AdSCs differentiated into adipocytes with over 80% of the cells showing accumulation of lipid vesicles at the end of 15 days. Transgene expression is maintained for over 10 days in dividing AdSCs and in differentiating AdSCs for 14 days. Global gene expression analysis of AdSC and differentiating adipocytes progressively became distinct with differentiation. Gene oncology analysis of the gene expression data identified several clusters of genes and key pathway genes within these clusters: like STAT1, ERK2, c-Jun, etc. The BacMam Ver 1 and Ver 2 (with WPRE element for prolonged expression and VSV-G cassette for wide range mammalian host cell infectivity) vectors with EBNA/OriP were used successfully to transducer H9 ESCs and HDF6-derived iPSC lines with over 50% transduction at 48 h post transduction. Expression of the transgene is maintained for over 10 days in dividing AdSC These vectors provide a delivery tool for constitutive or lineage-specific promoter driven genes (chemiluminescence, TR-FRTE, fluorescence reporters, toxicity screens) and response elements for high throughput screening imaging and assays.

Rat NSCs (Neural Stem Cells) were also efficiently transduced with BacMam Ver 1 and Ver 2 [SEQ ID NOs: 11 and 49] and transgene expression persists in differentiating astrocytes and oligodendrocytes for 6 days.

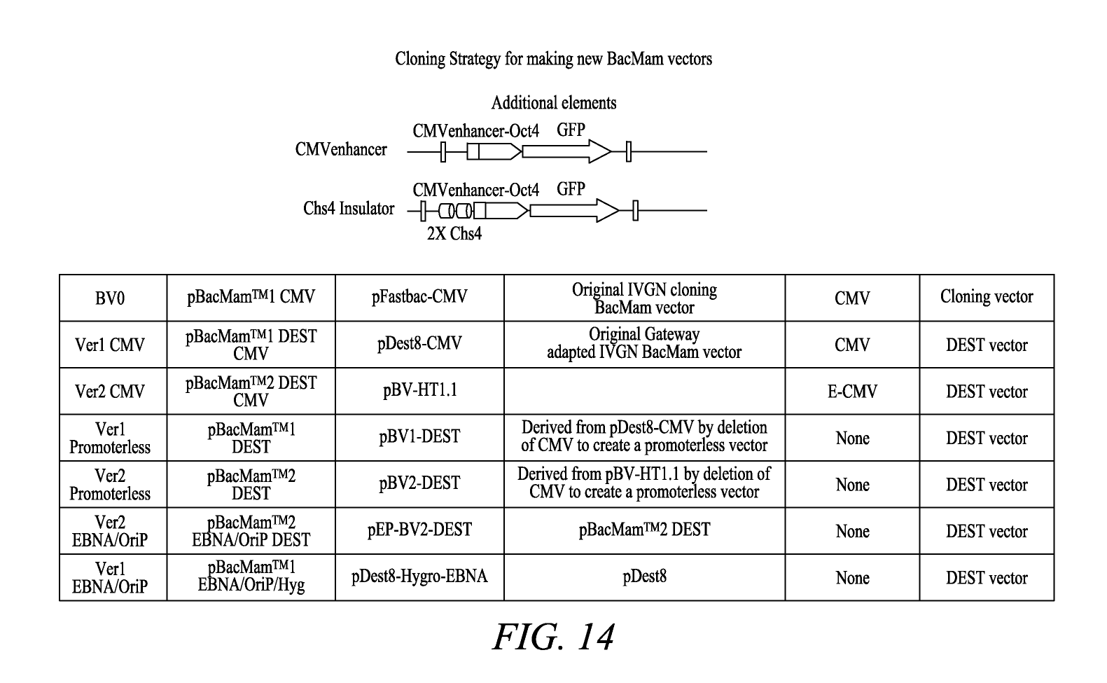

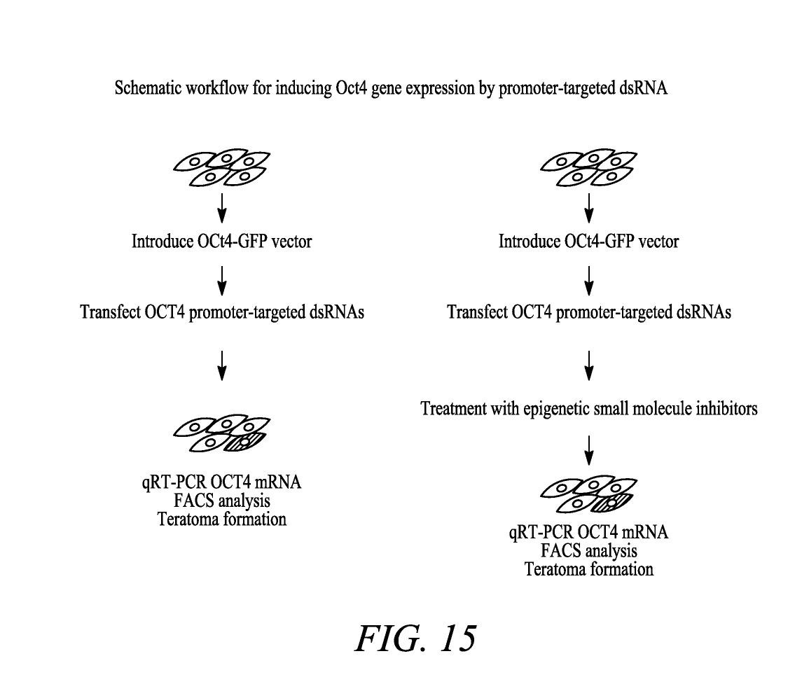

Further novel and hybrid vector systems are being developed (see FIG. 14) which will provide faster, efficient methods to create labeled stem cells for downstream therapeutic and screening applications: for e.g., to study basic cell biology and development pathways, to discover and evaluate drugs for the treatment of disease. BacMam vectors are being developed that use additional enhancer elements, or engineered enhancers, by altering epigenetic modulators for enhanced expression of transgenes (e.g.: insulators, introns, etc.) (FIG. 14) to better express and regulate reprogramming genes. These vector platforms will also allow us to generate embryonic and adult stem cells expressing transgenes of interest.

Most stem cells when driven towards differentiation result in a mixture of cells representative of various lineages. Methods of the invention may be useful to identify, label, or separate, specific cell types from a heterogeneous mixture of cells. For instance, when a lineage-specific promoter is used, differentiated cells that express the lineage-specific driven genes encoded by the vector can be distinguished from other non-expressing cells. The invention is applicable to the use of a Lineage Light BacMam system, which allows the identification, enrichment or isolation of any cell type of interest from a mixture of cells. For example, a liver specific promoter, such as AFP driving the expression of GFP can be used to identify embryonic stem cells that are differentiating into liver cells. The Lineage light reagent can be directly applied to cells during various stages of differentiation to detect the presence of a cell type of interest.

Although this embodiment discusses the use of components from the baculoviral backbone, components, or a combination of components from other viral backbones, such as adenoviral, lentiviral, retroviral, etc., which are known and practiced in the art, are also useful for the generation of such vectors.

### Example 3

### Reprogramming Normal Human Cells Using Transient BacMam Particles

Highly-efficient transient delivery of genes of interest into normal human cells using BacMam particles have been described. Sometimes, shorter (2-3 days) or longer (8-12 days) periods of transgene expression maybe best for reprogramming a certain type of cell. Multiple reprogramming genes (for e.g., Oct-4 and Sox-2) may be required to reprogram certain somatic cells like human fibroblasts, and the optimal length of expression of each of these genes to achieve ideal reprogramming needs to be determined. Here we describe the creation of BacMam particles (Ver 1 and 2 without EBNA/OriP) containing reprogramming genes like hOct4, hSox2, hKlf4, hcMyc, hNanog, and hLin28, and their efficient expression into somatic cells, for e.g., normal human skin cells, to generate iPSCs (induced pluripotent stem cells). Single or multiple treatments of cells with either BacMam viral constructs, or combinations thereof, may be required to achieve highly efficient reprogramming of cells.

Materials and Methods. BacMam particles containing the reprogramming genes hOct4, hSox2, hKlf4, hcMyc, hNanog, and hLin28 (BacRGs) were created as follows: the open reading frames of entry clones containing the genes were cloned into the expression vector pDEST8-CMV (version 1, Ver1 or v1 [SEQ ID NO: 2]). DNA from expression vector clones were used to transform DH10Bac E. coli which contain the baculovirus genome (BacMid). Recombinant BacMid DNA containing the gene of interest was purified and transfected into Sf9 insect cells yielding viral particles containing the gene of interest (P0). Viral particles were subjected to two rounds of amplification (P1, P2). Inserted gene integrity and viral purity of P2 preparations were confirmed by PCR and sequencing. hOct4 was also cloned into the ‘version 2, v2’ BacMam expression vector containing the VSV-G and WPRE sequences in addition to the CMV promoter and particles created as above. P2 viral particles were used to transduce normal human dermal fibroblasts (HDFs). At various times after transduction, cells were fixed for use in immunocytochemistry (ICC), or harvested for use in western immunoblots. A regimen of treatment of HDF with four ‘classic’ reprogramming genes hOct4, hSox2, hKlf4, hcMyc was developed. Putative iPSC colonies were selected and grown on feeder layers in StemPro ESC medium supplemented with Knockout Serum Replacement (KSR).

Results. Treatment of HDF with BacRGs resulted in expression of individual reprogramming genes in greater than 80% of treated cells (by ICC) and expression of the correct molecular weight protein was confirmed by western blot analysis. Expression of reprogramming proteins was transient and varied from 48-96 hours after single exposure to the BacRG particles. Cells could be treated with viral particles multiple times with repeated expression of the protein, however, the ability to express decreased with multiple treatments. In our first experiment, treatment of HDFs with four ‘classic’ reprogramming gene particles at intervals of 72 hours resulted in the development of colonies with stem cell morphology with 2× or 4× treatments. The colonies were successfully transferred to feeder layers, but stopped growing after two transfers.

The frequency of transduction and length of expression of the reprogramming gene(s) in target cells when the Ver1 [SEQ ID NO: 2] BacRGs are used could be a drawback to successful high frequency reprogramming. To determine if we could enhance the frequency of transduction and duration of gene expression, we cloned the hOct4 gene into the v2 expression vector and created viral particles. When cells were transduced with v2BacRG-hOct4 the dose (particles/cell) required for expression was reduced by 10-50 fold and the length of expression of the protein more than doubled when compared to v1BacRG-hOct4 particles.

Our results demonstrate that: Reprogramming genes can be successfully delivered into somatic cells like normal human fibroblasts using BacMam particles, and that the expression of the reprogramming gene can be constitutively controlled by the CMV promoter.

In the absence of an antibiotic resistance marker, expression of the genes delivered by Ver1 [SEQ ID NO: 2] particles decreases to undetectable levels 72-96 hours after treatment, making the expression transient.

By utilizing Ver1 [SEQ ID NO: 2] particles, somatic cells like human fibroblasts can be treated multiple times over the course of 10-12 days, resulting in expression/re-expression of the reprogramming genes.

When human fibroblasts are treated with V1) [SEQ ID NO: 2] reprogramming particles at intervals of 72 hours, 2× and 4× treatments resulted in the formation of colonies with stem cell-like characteristics.

The inclusion of the VSV-G sequence in the BacMam vector (Ver 2) [SEQ ID NO: 8] significantly enhances the ability of the virus to enter human fibroblasts. i.e., the number of particles required to obtain the same number of transduced cells is reduced by 10-50 fold.

Inclusion of the WPRE element in the BacMam vector significantly increases the length of time that a reprogramming gene can be expressed in human fibroblasts.

Discussion: In cases where transient expression of a gene, for e.g., a reprogramming gene is more suitable, to select a pathway of differentiation, and where repeat treatments of the reprogramming gene may be necessary based on the expression level of the reprogramming product, transient expression using a vector without EBNA/Ori P may be desirable, as shown here. In the current studies we show that expression of reprogramming genes driven by the CMV promoter is transient and that repeated treatment with these viral constructs is possible without acutely deleterious effects on cell viability. In some cases, transient (several days) expression of reprogramming genes may provide the desired outcome more effectively than longer term expression maintained by the EBNA/OriP constructs. Single treatments with high doses of the virus (500-1000 particles per cell) result in approximately 80% of the cells in a culture expressing the Oct-4 or Sox-2 genes.

Inclusion of the VSV-G sequence (in the V2 construct: [SEQ ID NO: 8]) may enhance the ability of the baculovirus to transduce human fibroblasts. Thus, nearly 100% of the cells in a culture can be transduced effectively with 10-100 viral particles/cell. In this regard, the benefits of using the V2 construct include reduced production costs and reduced viral load which should minimize non-specific effects of treatment with the virus.

Inclusion of the WPRE element may greatly enhance the length of time that the Oct-4 gene is expressed in human fibroblasts. Expression of the protein encoded by the Oct-4 construct could be detected for at least 10 days after a single treatment with the V2 Oct-4 construct. Thus, longer expression times may be achieved by including this element.

### Example 4

### Transcriptional Gene Activation System

These experiments were performed to investigate that enhance reprogramming efficiency. Specifically, experiments were performed to investigate whether small promoter-targeted dsRNAs (double stranded RNAs) induce Transcriptional Gene Activation (TGA) of any or all of the four required genes (Oct4, Sox2, c-Myc and Klf4) in adult stem cells. Various custom designed dsRNAs (21mer), as shown in Attachment P, which target specific regions of the Oct4 promoter gene, were transiently transfected into stem cells to see if they affect transcription. The workflow for these experiments is shown in Attachment O. Specifically, some of these experiments were designed to determine whether: (i) induction levels triggered by promoter-targeted dsRNAs of the reprogramming factors induce pluripotency, (ii) the duration of TGA directly correlates with reprogramming efficiency, (iii) different cell types require different induction levels triggered by TGA of targeted genes, and (iv) small molecules involved in chromatin modification, such as histone deacetylase (HDAC) inhibitors, have any effect on reprogramming.

Proof of Principle: Transcriptional Gene Activation (TGA) of the Oct-4 promoter (Attachment O)

The Invitrogen generated pEP-hOG vector which drives GFP expression (13,588 bp), (Attachment F) was utilized to measure OCT4 promoter driven gene expression.

Vector pEP-hOG was introduced into embryonic fibroblasts. This was done to generate an embryonic stem cell line expressing a stably integrated, single copy of Oct4-GFP.

Various custom designed dsRNAs (shown in Attachment P) that target specific regions of the OCT4 promoter were transiently transfected, or introduced using peptide delivery systems like MPG into the GFP expressing fibroblast cells (see Attachment O and steps in Rational Design Approach below).

In parallel, cells in steps 2 or 3 were treated with small molecules that are involved in chromatin modifications, to determine whether altering the epigenetic landscape of the targeted promoters facilitates or inhibits TGA (see below for small molecules involved in chromatin modifications).

The reprogramming efficiency of the dsRNA was quantified by measuring OCT4 mRNA levels using quantitative RT-PCR or by quantifying OCT4 GFP using FACS (see below for quantification of reprogramming efficiency).

Rational Design Approach for Promoter-Targeted dsRNAs Mediating transcriptional Gene Activation

Accession numbers were obtained and entered into DBTSS. “DBTSS defines putative promoter groups by clustering TSSs within a 500 bases intervals. DBTSS also provides detailed comparison between sequences around any user-specified pair of TSSs.”

Promoter sequences from step one were entered into the Transcription Factor Search Database to obtain conserved transcription factor motifs for gene(s) of interest.

Locations of the TSS and specific transcription factor binding sites were annotated and promoter-targeted duplex RNAs were designed. Regions with high GC content were avoided; preferred length was 21mer, and promoter context.

dsRNAs thus generated and shown in Attachment P were delivered to cells of interest. Validation and Functional Assays that were developed are discussed below.

TABLE 2
ds RNA mers For Reprogramming Cells

| | | Scale | | | | |
|---|---|---|---|---|---|---|
| | Sequence (enter all | of | 5′ | 3′ | | Special |
| RNA Name | sequences 5′ to 3′) | Syn | Mods | Mods | Purity | Codes |
| OCT4 dsRNA | GCAUUGAGGGAUAGCGCCACA | 20N | # | # | DSL | B |
| mir147 (SEQ ID. | CACTT | | | | | |
| No: 13) | | | | | | |
| OCT4 dsRNA | GUGUGUGGCGCUAUCCCUCAA | 20N | # | # | DSL | B |
| mir147 (SEQ ID. | UGCTT | | | | | |
| No: 14) | | | | | | |
| OCT4 dsRNA | AAAAAGUUUCUGUGGGGGACC | 20N | # | # | DSL | B |
| mir148a (SEQ ID. | UGCACUGATT | | | | | |
| No: 15) | | | | | | |
| OCT4 dsRNA | UCAGUGCAGGUCCCCCACAGA | 20N | # | # | DSL | B |
| mir148a (SEQ ID. | AACUUUUUTT | | | | | |
| No: 16) | | | | | | |
| OCT4 dsRNA | CCCCUGAAGGCACAGUGCCAG | 20N | # | # | DSL | B |
| mir149 (SEQ ID. | ATT | | | | | |
| No: 17) | | | | | | |
| OCT4 dsRNA | UCUGGCACUGUGCCUUCAGGG | 20N | # | # | DSL | B |
| mir149 (SEQ ID. | GTT | | | | | |
| No: 18) | | | | | | |
| OCT4 dsRNA | GGCCAGGGGGGCCGGAGCCGG | 20N | # | # | DSL | B |
| mir149TSS (SEQ | GTT | | | | | |
| ID. No: 19) | | | | | | |
| OCT4 dsRNA | CCCGGCUCCGGCCCCCCUGGCC | 20N | # | # | DSL | B |
| mir149TSS (SEQ | TT | | | | | |
| ID. No: 20) | | | | | | |
| OCT4 dsRNA | GCCAGGGAGCGGGUUGGGAGU | 20N | # | # | DSL | B |
| mir150 (SEQ ID. | TT | | | | | |
| No: 21) | | | | | | |
| OCT4 dsRNA | ACUCCCAACCCGCUCCCUGGCT | 20N | # | # | DSL | B |
| mir150 (SEQ ID. | T | | | | | |
| No: 22) | | | | | | |
| OCT4 dsRNA | GUGGCUGGAUUUGGCCAGUAT | 20N | # | # | DSL | B |
| mir193b (SEQ ID. | T | | | | | |
| No: 23) | | | | | | |
| OCT4 dsRNA | UACUGGCCAAAUCCAGCCACT | 20N | # | # | DSL | B |
| mir193b (SEQ ID. | T | | | | | |
| No: 24) | | | | | | |
| OCT4 dsRNA | CCAGGGGGCGGGGCCAGTT | 20N | # | # | DSL | B |
| mir296 (SEQ ID. | | | | | | |
| No: 25) | | | | | | |
| OCT4 dsRNA | CUGGCCCCGCCCCCUGGTT | 20N | # | # | DSL | B |
| mir296 (SEQ ID. | | | | | | |
| No: 26) | | | | | | |
| OCT4 dsRNA | GGAGGAUUUCUUGAGGACAGG | 20N | # | # | DSL | B |
| mir339 (SEQ ID. | AATT | | | | | |
| No: 27) | | | | | | |
| OCT4 dsRNA | UUCCUGUCCUCAAGAAAUCCU | 20N | # | # | DSL | B |
| mir339 (SEQ ID. | CCTT | | | | | |
| No: 28) | | | | | | |
| OCT4 dsRNA | UUUGGCAGGCUGGGCAGAUGT | 20N | # | # | DSL | B |
| mir346 (SEQ ID. | T | | | | | |
| No: 29) | | | | | | |
| OCT4 dsRNA | CAUCUGCCCAGCCUGCCAAAT | 20N | # | # | DSL | B |
| mir346 (SEQ ID. | T | | | | | |
| No: 30) | | | | | | |
| OCT4 dsRNA | UGAAGAACAUGGAGGUGUGGG | 20N | # | # | DSL | B |
| mir483 (SEQ ID. | AGUGATT | | | | | |
| No: 31) | | | | | | |
| OCT4 dsRNA | UGAAGAACAUGGAGGUGUGGG | 20N | # | # | DSL | B |
| mir483 (SEQ ID. | AGUGATT | | | | | |
| No: 23) | | | | | | |
| OCT4 dsRNA | GCUGGGAUGUGCAGAGCCUGA | 20N | # | # | DSL | B |
| mir484 (SEQ ID. | TT | | | | | |
| No: 33) | | | | | | |
| OCT4 dsRNA | UCAGGCUCU GC | 20N | # | # | DSL | B |
| mir484 (SEQ ID. | ACAUCCCAGCTT | | | | | |
| No: 34) | | | | | | |
| OCT4 dsRNA | GAGGGAUAGCGCCACACACTT | 20N | # | # | DSL | B |
| mir147(21mer) | | | | | | |
| (SEQ ID. No: 35) | | | | | | |
| OCT4 dsRNA | GUGUGUGGCGCUAUCCCUCTT | 20N | # | # | DSL | B |
| mir147(21mer) | | | | | | |
| (SEQ ID. No: 36) | | | | | | |
| OCT4 dsRNA | UGUGGGGGACCUGCACUGATT | 20N | # | # | DSL | B |
| mir148a(21mer) | | | | | | |
| (SEQ ID. No: 37) | | | | | | |
| OCT4 dsRNA | UCAGUGCAGGUCCCCCACATT | 20N | # | # | DSL | B |
| mir148a(21mer) | | | | | | |
| (SEQ ID. No: 38) | | | | | | |
| OCT4 dsRNA | CUGAAGGCACAGUGCCAGATT | 20N | # | # | DSL | B |
| mir149(21mer) | | | | | | |
| (SEQ ID. No: 39) | | | | | | |
| OCT4 dsRNA | UCUGGCACUGUGCCUUCAGTT | 20N | # | # | DSL | B |
| mir149(21mer) | | | | | | |
| (SEQ ID. No: 40) | | | | | | |
| OCT4 dsRNA | CAGGGAGCGGGUUGGGAGUTT | 20N | # | # | DSL | B |
| mir150(21mer) | | | | | | |
| (SEQ ID. No: 41) | | | | | | |
| OCT4 dsRNA | ACUCCCAACCCGCUCCCUGTT | 20N | # | # | DSL | B |
| mir150(21mer) | | | | | | |
| (SEQ ID. No: 42) | | | | | | |
| OCT4 dsRNA | GAUUUCUUGAGGACAGGAATT | 20N | # | # | DSL | B |
| mir339(21mer) | | | | | | |
| (SEQ ID. No: 43) | | | | | | |
| OCT4 dsRNA | UUCCUGUCCUCAAGAAAUCTT | 20N | # | # | DSL | B |
| mir339(21mer) | | | | | | |
| (SEQ ID. No: 44) | | | | | | |
| OCT4 dsRNA | CAUGGAGGUGUGGGAGUGATT | 20N | # | # | DSL | B |
| mir483(21mer) | | | | | | |
| (SEQ ID. No: 45) | | | | | | |
| OCT4 dsRNA | UGAAGAACAUGGAGGUGUGTT | 20N | # | # | DSL | B |
| mir483(21mer) | | | | | | |
| (SEQ ID. No: 46) | | | | | | |
| Oct4dsRNA | AUAAAAAAACUAACAGGGCTT | 20N | # | # | DSL | B |
| 111(21mer) (SEQ | | | | | | |
| ID. No: 47) | | | | | | |
| Oct4dsRNA | GCCCUGUUAGUUUUUUUAUTT | 20N | # | # | DSL | B |
| 111(21mer) (SEQ | | | | | | |
| ID. No: 48) | | | | | | |

Use of Small Molecules Involved in Chromatin Modifications:

The following chemicals were tested: 5′-azaC from Sigma-Aldrich, SAHA from Biomol International, dexamethasone, TSA, and VPA from EMD Biosciences.

Stock solutions of 5′-azaC and VPA were made in PBS or media. Stock solutions of other chemicals were made in DMSO.

Quantification of Reprogramming Efficiency

Two methods were initially used to quantify reprogramming efficiency. (1) FACS analysis to quantify the induction of Oct4-GFP+ cells. Also the number of Oct4-GFP+ cells induced at different time points were counted directly under a fluorescent microscope or a fluorescent dissection microscope. (2) Gene expression analysis: mRNA was isolated using mRNA catcher plate and Oct4, Sox2, c-Myc and Klf4 mRNA levels were measured quantitatively by qRTPCR methods.

Generation of Teratomas

Teratomas were produced by injecting ˜1 million cells subcutaneously into NODSCID mice. Tumor samples were collected with in 5 weeks, fixed in 4% paraformaldehyde and processed for paraffin embedding and hematoxylin and eosin staining following standard procedures.

All references cited throughout the disclosure are hereby expressly incorporated by reference.

1. An isolated nucleic acid molecule comprising (a) an OriP site, (b) a DNA segment encoding the EBNA1 gene; (c) one or more att recombination sites; and (d) a DNA segment encoding at least one selectable marker.

2. The isolated nucleic acid molecule of claim 1 wherein EBNA1 expression is constitutive.

3. The isolated nucleic acid molecule of claim 2 wherein the constitutive promoter driving EBNA1 expression is selected from a group consisting of the native EBNA1 promoter, a strong viral promoter, an engineered constitutive promoter or a constitutive lineage or tissue-specific promoter.

4. The isolated nucleic acid molecule of claim 1 wherein EBNA1 expression is inducible.

5. The isolated nucleic acid molecule of claim 4 wherein the inducible promoter driving EBNA1 expression is an inducible antibiotic operon.

6. The isolated nucleic acid molecule of claim 1, further comprising one or more expression cassettes, each containing a promoter operably linked to a DNA sequence for which expression is desired.

7. The isolated nucleic acid molecule of claim 6 wherein the expression cassette encodes for a tissue-specific gene, reprogramming gene or a developmental gene.

8. The isolated nucleic acid molecule of claim 7 wherein the reprogramming gene is selected from a group consisting of Oct4, Sox2, c-Myc, Klf4, Oct3/4, Nanog, Lin28, SSEA1, and TRA1-80.

9. The isolated nucleic acid molecule of claim 6 wherein the promoter driving the expression cassette is of a type selected from a group consisting of cell-specific promoters, tissue-specific promoters, reprogramming gene promoters, and developmental gene promoters.

10-11. (canceled)

12. The isolated nucleic acid molecule of claim 9 wherein the promoter driving the expression cassette is a cell-specific promoter.

13. The isolated nucleic acid molecule of claim 9 wherein the promoter driving the expression cassette is a developmental stage-specific promoter.

14. The isolated nucleic acid molecule of claim 12 wherein the cell is a stem cell.

15. The isolated nucleic acid molecule of claim 13 wherein the developmental stage is either a germ, embryonic, progenitor, fetal, neonatal, or stem cell stage.

16. The isolated nucleic acid molecule of claim 10 wherein the mammal is human.

17. The isolated nucleic acid molecule of claim 9 wherein the reprogramming gene promoter is selected from a group of promoters consisting of Oct4, Sox2, c-Myc, Klf4, Oct3/4, Nanog, Lin28, SSEA1, and TRA1-80.

18. The isolated nucleic acid molecule of claim 1 wherein the selectable marker is either a fluorescent protein, a protein that confers antibiotic resistance, or an enzyme.

19-64. (canceled)

65. A cell transduced with one or a combination of nucleic acid molecules of claim 7, each nucleic acid molecule carrying at least one expression cassette for reprogramming said cell.

66-73. (canceled)

74. A method for reprogramming cells comprising:
(i) introducing one or a combination of the nucleic acid molecules of claim 7 into a cell;
(ii) expressing one or more polypeptides encoded thereof in said cell under appropriate culturing conditions;
(iii) identifying whether said cell has been reprogrammed.

75-92. (canceled)

93. A method for producing an induced pluripotent cell (iPSC) comprising:
(i) introducing one or a combination of the nucleic acid molecules of claim 7 into a cell;
(ii) expressing one or more polypeptides encoded thereof in said cell under appropriate culturing conditions;
(iii) identifying whether said cell has been reprogrammed.

94-98. (canceled)


SEQUENCE LISTING (50 sequences)

SEQ ID NO: 1 - DNA, 10186 bp, Artificial Sequence
  pCEP4
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagcaagcttgctagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 2 - DNA, 7280 bp, Artificial Sequence
  pBacMam Ver1
ctcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggcggccgctaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggcta

SEQ ID NO: 3 - DNA, 6671 bp, Artificial Sequence
  pBacMam Ver1 promoterless
ctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggcggccgctaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttcttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctactcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 4 - DNA, 10641 bp, Artificial Sequence
  plasmid pEBNA-DEST
tcgactagcttggcacgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgatccggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggatcgatcctctagagtcgactagtaacggccgccagtgtgctggaattcggcttacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcggctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggcttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtaagccgaattctgcagat

SEQ ID NO: 5 - DNA, 11563 bp, Artificial Sequence
  plasmid pEBNA-DEST  with EF1a-GFP
ttgtacaaacttgtaagccgaattccagcacactggcggccgttactagtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaatctgcagaattcggcttaccactttgtacaagaaagctgggtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcactagctcctgtgtataatattttcataaatcatactcagtaagcaaaactctcaagcagcaagcatatgcagctagtttaacacattatacacttaaaaattttatatttaccttagagctttaaatctctgtaggtagtttgtccaattatgtcacaccacagaagtaaggttccttcacaaagatcccaagctagcagttttcccagtcacgacgttgtaaaacgacggccagtgcctagcttataatacgactcactatagggagagagctatgacgtcgcatgcacgcgtaagcttgggccctctagagcggccgctcactattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtagcaacttttgtatacaaagttgctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctaagcctgctttt

SEQ ID NO: 6 - DNA, 13588 bp, Artificial Sequence
  plasmid pEBNA-DEST  with Oct4-GFP
ttgtacaaacttgtaagccgaattccagcacactggcggccgttactagtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaatctgcagaattcggcttaccactttgtacaagaaagctgggtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcactagctcctgtgtataatattttcataaatcatactcagtaagcaaaactctcaagcagcaagcatatgcagctagtttaacacattatacacttaaaaattttatatttaccttagagctttaaatctctgtaggtagtttgtccaattatgtcacaccacagaagtaaggttccttcacaaagatcccaagctagcagttttcccagtcacgacgttgtaaaacgacggccagtgcctagcttataatacgactcactatagggagagagctatgacgtcgcatgcacgcgtaagcttgggccctctagagcggccgctcactattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtagcaacttttgtatacaaagttgtggggaaggaaggcgccccaagccgggggcctggtgaaatgagggcttgcgaagggactactcaacccctctctccctccccagtcccacccactagccttgacctctggccccgccccctggatgggtggaggagagggaggtggggggagaaactgaggcgaaggatgtttgcctaatggtggtggcaatggtgtctgtggaaggggaaaaccgggagacacaactggcgcccctccaggacctcagtgcaggtcccccacagaaactttttttatttttattttttaagacagggtctcactttgttgcccagactggagtgcagtggagtacaatgatggctcaatgtagcctcgatctactgggccaaagcaatccttctgctccagcctcctaattggctgggactacaggcttggaccactgtgccctgttagtttttttatttttagtagagatggggccttgctatgttacccaggctggtcttgaattcctgtcctcaagaaatcctcccacctctgccgcccagtgtcatgattaaaggcgtgagccaccacacccaactttcaactcccaacccgctccctggcactctctcaggctctgcacatcccagctgtctggaatcactcccacaactccatgttcttcaggaacccaggtgcttgaccccctctccacagacctctggcactgtgccttcaggggccagtcaccctctcagctcctcaaatttattgaatgtgtgtgtggcgctatccctcaatgcatcaacagccataagcacaatggccagctgctcccttatgccttcccccgatccatccagaatcctaggcattcccatcccgatactggccaaatccagccaccccgcagcctgggtgcctggcaccatctgcccagcctgccaaatttcaccccatcttcaagagtagactgccagacaaggcctccgtgctatatccccccacccccccatccccccacccctccgtcttccagaatcagactccagactctcctcatctaacagactaaggggttggtccctacttccccttcaagggaccagactttggactgactgggcctcagtttcccaacctttgctgaaacagagtgataagacacccgctttgggccccctccactatggaacctgcacatcaggttccttgctcccctctcaaccaaaactcagacatctaataccacggtaggccccgttctccctcccccacctccctggcccaggcctccagccctaggccctgggtggggaaaaccagggggtggggggtgtggagaaaaaatatctgacttcaggttcaaagaagcctgggagggactgggggaagggggcaggacaatggccttggctggacaatcccggtccccagagggggcagctctaaccctaaacaagtgctcaacccttgaatgggcctggatggctcccctggggactgcttcctgctccccaaccccccagtcccaatcccctcacacagaatccccttcagagacgctaaaaggagctccagcaacccccctctgcaatcccctcaaagactgagcctcagacgggcaccaagggccccccacagggacctaggtatctagttcctccttcctctgggggactcaggcgtccagcttcatcgtgcgtccctccccgagcctggcagattgagggatgtgctttgtttagtggggctggctggcagaaagacgcagaggaggtggagagtgatttgtggaggcgtgcaggaaggctgccctaagctccccttcagggtctgtttttctgggcctggcctgagtatcctgaggctcatgctgctggtctagtgcttgattctgtttgcaagagaatagccaacggaatgcctgtctgtgagggatgatgtttgtctgtctgctcccaaaacttgatctcagtggagggcctggggtaagtctgggggctccagagggggctctgggccagggctccccacagcttcgaaggccagaaggccaggtctggactgggcacgctgacctctgtcgacttaagtaaggcttctcattgcaggctccaggctcagccctgcctgggcttgtctgctgaggtcagtggctctatctgccttctaaggggatgggtgtcccgtggccagctgtcttcatcttggtggcatccgtgagtcttttgagacttttcccccactcttatgttgcctctgttcgtgtgcccatctcctgtctgtgtagactttttgagcctaattgtatgcgtgcatttcaatacctgccacaggtctgccggaaggtctacaaggcagtggggttgcagctgtgttcacttctcggcctttaactgcccaaaaggcaggtagattatggggcctggtgggggtaggaggaacatgcttcggaacaggaggaggcccctccccagccatctcaatccccaggacagaaccatcacggcacctttgtcatgcatctctctgctgtctgccaagaagacggcctctcagaggagggggaggggcaggcctgggatttggctggaatctccacaccagtgtttctcagcttgccatcctccaggttccccaaaagcgctcttcccaagccagtccagagagtccctgctgcccattttcctagtggctcctaaaacaccttccccaatttccccactcaacaccaccctcttgtttttagattataatttgtactgtaggtggtgtatttctggcctgggcaagaggcccattcccgagagggacgcagacaaggggtgggtgcctgggtccctggctgccttgtggctggatatgagcccagtcaggggtcagcctcctgcatgcctagactcctagccggcccccttctggggtgctcagggctgatgggaggttgaggcaggctttccttccttctcactgtcctgttatgcctgaagggtaggtggcttcacttcagccaaggccagctctcccaggccccaaccagtgctgggggccaccgttgggcctggaggagactggaagccaggctgagtcatcagaactggtcccatgattccctgggttttagaaagtcaccataaaaagatacttcacacacacctttattattacagtgcaatgtcaagacccttcacagagcactgccaggggacccaggtgaggcccacctctcctaagcctgctttt

SEQ ID NO: 7 - DNA, 15523 bp, Artificial Sequence
  pBacMam Ver1 with Tet Operon and EBNA/Ori P
gtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatgcaatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtacccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgaatccatcacactggcggccgctcgagcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatagcctgcttttttgtacaaacttgttgatatctgcagaattccaccacactggactagtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactacccggtcatcatcaccatcaccattgagtttaaacccgctgatcagcctcgactgtgccttctagttgccagccatctgttgtttgcccctcccccgtgccttccttgaccctggaaggtgccactcccactgtcttcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaatctgcagaattcggcttaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtaagccgaattccagcacactggcggccgttactagtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttcctcaaattcctcgtccccctttttgctggacggtagggatggggattctcgggacccctcctcttcctcttcaaggtcaccttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcag

SEQ ID NO: 8 - DNA, 9762 bp, Artificial Sequence
  pBacMam Ver2
aattgttgttgttaacttgtttattgcagcttataatggttacaaataaagcaatagcatcacaaatttcacaaataaagcatttttttcactgcattctagttgtggtttgtccaaactcatcaatgtatcttaagactagtgagctcgtcgacgtaggcctttgaattccgcgcgcttcggaccgggatccctcgaggaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcctcgagggatccgcgcccgatggtgggacggtatgaataatccggaatatttataggtttttttattacaaaactgttacgaaaacagtaaaatacttatttatttgcgagatggttatcattttaattatctccatgatctattaatattccggagtatacgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagccttaattaaggcatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgaattctgcagtcgacggtaccgcgggcccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgggatccaccgggtacaagtaaagcggccgcgactctagatcataatcagccataccacatttgtagaggttttacttgctttaaaaaacctcccacacctccccctgaacctgaaacataaaatgaatgcaattgtttcccgttatttgcactctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcggcgtccggtccgtgttgcttggtcttcacctgtgcagacttgcgaaccatggattc

SEQ ID NO: 9 - DNA, 8830 bp, Artificial Sequence
  pBacMam Ver2-CMV-GFP
ttgtacaaagtggtgatgggatccaccgggtacaagtaaagcggccgcgactctagatcataatcagccataccacatttgtagaggttttacttgctttaaaaaacctcccacacctccccctgaacctgaaacataaaatgaatgcaattgtttcccgttatttgcactctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcggcgtccggtccgtgttgcttggtcttcacctgtgcagacttgcgaaccatggattcaattgttgttgttaacttgtttattgcagcttataatggttacaaataaagcaatagcatcacaaatttcacaaataaagcatttttttcactgcattctagttgtggtttgtccaaactcatcaatgtatcttaagactagtgagctcgtcgacgtaggcctttgaattccgcgcgcttcggaccgggatccctcgaggaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcctcgagggatccgcgcccgatggtgggacggtatgaataatccggaatatttataggtttttttattacaaaactgttacgaaaacagtaaaatacttatttatttgcgagatggttatcattttaattatctccatgatctattaatattccggagtatacgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagccttaattaaggcatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgaattctgcagtcgacggtaccgcgggcccatcacaagtttgtacaaaaaagcaggcttaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatacccagctttc

SEQ ID NO: 10 - DNA, 8851 bp, Artificial Sequence
  pBacMam Ver2- promoterless
agcttcgaattctgcagtcgacggtaccgcgggcccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgggatccaccgggtacaagtaaagcggccgcgactctagatcataatcagccataccacatttgtagaggttttacttgctttaaaaaacctcccacacctccccctgaacctgaaacataaaatgaatgcaattgtttcccgttatttgcactctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcggcgtccggtccgtgttgcttggtcttcacctgtgcagacttgcgaaccatggattcaattgttgttgttaacttgtttattgcagcttataatggttacaaataaagcaatagcatcacaaatttcacaaataaagcatttttttcactgcattctagttgtggtttgtccaaactcatcaatgtatcttaagactagtgagctcgtcgacgtaggcctttgaattccgcgcgcttcggaccgggatccctcgaggaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcctcgagggatccgcgcccgatggtgggacggtatgaataatccggaatatttataggtttttttattacaaaactgttacgaaaacagtaaaatacttatttatttgcgagatggttatcattttaattatctccatgatctattaatattccggagtatacgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagcctta

SEQ ID NO: 11 - DNA, 13708 bp, Artificial Sequence
  pBacMam Ver2- promoterless with EBNA/Ori P
gtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagccttaagcttcgaattctgcagtcgacggtaccgcgggcccatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatgggatccaccgggtacaagtaaagcggccgcgactctagatcataatcagccataccacatttgtagaggttttacttgctttaaaaaacctcccacacctccccctgaacctgaaacataaaatgaatgcaattgtttcccgttatttgcactctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcggcgtccggtccgtgttgcttggtcttcacctgtgcagacttgcgaaccatggattcaattgttgttgttaacttgtttattgcagcttataatggttacaaataaagcaatagcatcacaaatttcacaaataaagcatttttttcactgcattctagttgtggtttgtccaaactcatcaatgtatcttaagactagtgagctcgtcgacgtaggcctttgaattccgcgcgcttcggaccgggatccctcgaggaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcctcgagggatccgcgcccgatggtgggacggtatgaataatccggaatatttataggtttttttattacaaaactgttacgaaaacagtaaaatacttatttatttgcgagatggttatcattttaattatctccatgatctattaatattccggagtataccgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcg

SEQ ID NO: 12 - DNA, 7883 bp, Artificial Sequence
  pBacMam Ver2 with Tet Operon
ttgtacaaagtggtgatgggatccaccgggtacaagtaaagcggccgcgactctagatcataatcagccataccacatttgtagaggttttacttgctttaaaaaacctcccacacctccccctgaacctgaaacataaaatgaatgcaattgtttcccgttatttgcactctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcggcgtccggtccgtgttgcttggtcttcacctgtgcagacttgcgaaccatggattcaattgttgttgttaacttgtttattgcagcttataatggttacaaataaagcaatagcatcacaaatttcacaaataaagcatttttttcactgcattctagttgtggtttgtccaaactcatcaatgtatcttaagactagtgagctcgtcgacgtaggcctttgaattccgcgcgcttcggaccgggatccctcgaggaattccgtttttttttttttttttcataaaaattaaaaactcaaatataattgaggcctctttgagcatggtatcacaagttgatttggtccaaacatgaagaatctgttgtgcaggatttgagttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcatagtgtcagaattcctcgagggatccgcgcccgatggtgggacggtatgaataatccggaatatttataggtttttttattacaaaactgttacgaaaacagtaaaatacttatttatttgcgagatggttatcattttaattatctccatgatctattaatattccggagtatacgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagccttaagcttcgaattctgcagtcgacggtaccgcgggcccatcacaagtttgtacaaaaaagcaggctataacttcgtataatgtatgctatacgaagttatccgcttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgactagtccagtgtggtggaattctgcagatatcaacaagtttgtacaaaaaagcaggcaccataacttcgtataatgtatgctatacgaagttattacccagctttc

SEQ ID NO: 13 - DNA, 26 bp, Artificial Sequence
  OCT4 dsRNA mir147
gcauugagggauagcgccacacactt

SEQ ID NO: 14 - DNA, 26 bp, Artificial Sequence
  OCT4 dsRNA mir147
guguguggcgcuaucccucaaugctt

SEQ ID NO: 15 - DNA, 31 bp, Artificial Sequence
  OCT4 dsRNA mir148a
aaaaaguuucugugggggaccugcacugatt

SEQ ID NO: 16 - DNA, 31 bp, Artificial Sequence
  OCT4 dsRNA mir148a
ucagugcaggucccccacagaaacuuuuutt

SEQ ID NO: 17 - DNA, 24 bp, Artificial Sequence
  OCT4 dsRNA mir149
ccccugaaggcacagugccagatt

SEQ ID NO: 18 - DNA, 24 bp, Artificial Sequence
  OCT4 dsRNA mir149
ucuggcacugugccuucaggggtt

SEQ ID NO: 19 - DNA, 24 bp, Artificial Sequence
  OCT4 dsRNA mir149TSS
ggccaggggggccggagccgggtt

SEQ ID NO: 20 - DNA, 24 bp, Artificial Sequence
  OCT4 dsRNA mir149TSS
cccggcuccggccccccuggcctt

SEQ ID NO: 21 - DNA, 23 bp, Artificial Sequence
  OCT4 dsRNA mir150
gccagggagcggguugggagutt

SEQ ID NO: 22 - DNA, 23 bp, Artificial Sequence
  OCT4 dsRNA mir150
acucccaacccgcucccuggctt

SEQ ID NO: 23 - DNA, 22 bp, Artificial Sequence
  OCT4 dsRNA mir193b
guggcuggauuuggccaguatt

SEQ ID NO: 24 - DNA, 22 bp, Artificial Sequence
  OCT4 dsRNA mir193b
uacuggccaaauccagccactt

SEQ ID NO: 25 - DNA, 19 bp, Artificial Sequence
  OCT4 dsRNA mir296
ccagggggcggggccagtt

SEQ ID NO: 26 - DNA, 19 bp, Artificial Sequence
  OCT4 dsRNA mir296
cuggccccgcccccuggtt

SEQ ID NO: 27 - DNA, 25 bp, Artificial Sequence
  OCT4 dsRNA mir339
ggaggauuucuugaggacaggaatt

SEQ ID NO: 28 - DNA, 25 bp, Artificial Sequence
  OCT4 dsRNA mir339
uuccuguccucaagaaauccucctt

SEQ ID NO: 29 - DNA, 22 bp, Artificial Sequence
  OCT4 dsRNA mir346
uuuggcaggcugggcagaugtt

SEQ ID NO: 30 - DNA, 22 bp, Artificial Sequence
  OCT4 dsRNA mir346
caucugcccagccugccaaatt

SEQ ID NO: 31 - DNA, 28 bp, Artificial Sequence
  OCT4 dsRNA mir483
ugaagaacauggaggugugggagugatt

SEQ ID NO: 32 - DNA, 28 bp, Artificial Sequence
  OCT4 dsRNA mir483
ugaagaacauggaggugugggagugatt

SEQ ID NO: 33 - DNA, 23 bp, Artificial Sequence
  OCT4 dsRNA mir484
gcugggaugugcagagccugatt

SEQ ID NO: 34 - DNA, 12 bp, Artificial Sequence
  OCT4 dsRNA mir484
acaucccagctt

SEQ ID NO: 35 - DNA, 21 bp, Artificial Sequence
  OCT4 dsRNA mir147(21mer)
gagggauagcgccacacactt

SEQ ID NO: 36 - DNA, 21 bp, Artificial Sequence
  OCT4 dsRNA mir147(21mer)
guguguggcgcuaucccuctt

SEQ ID NO: 37 - DNA, 21 bp, Artificial Sequence
  OCT4 dsRNA mir148a(21mer)
ugugggggaccugcacugatt

SEQ ID NO: 38 - DNA, 21 bp, Artificial Sequence
  OCT4 dsRNA mir148a(21mer)
ucagugcaggucccccacatt

SEQ ID NO: 39 - DNA, 21 bp, Artificial Sequence
  OCT4 dsRNA mir149(21mer)
cugaaggcacagugccagatt

SEQ ID NO: 40 - DNA, 21 bp, Artificial Sequence
  OCT4 dsRNA mir149(21mer)
ucuggcacugugccuucagtt

SEQ ID NO: 41 - DNA, 21 bp, Artificial Sequence
  OCT4 dsRNA mir150(21mer)
cagggagcggguugggagutt

SEQ ID NO: 42 - DNA, 21 bp, Artificial Sequence
  OCT4 dsRNA mir150(21mer)
acucccaacccgcucccugtt

SEQ ID NO: 43 - DNA, 21 bp, Artificial Sequence
  OCT4 dsRNA mir339(21mer)
gauuucuugaggacaggaatt

SEQ ID NO: 44 - DNA, 21 bp, Artificial Sequence
  OCT4 dsRNA mir339(21mer)
uuccuguccucaagaaauctt

SEQ ID NO: 45 - DNA, 21 bp, Artificial Sequence
  OCT4 dsRNA mir483(21mer)
cauggaggugugggagugatt

SEQ ID NO: 46 - DNA, 21 bp, Artificial Sequence
  OCT4 dsRNA mir483(21mer)
ugaagaacauggaggugugtt

SEQ ID NO: 47 - DNA, 21 bp, Artificial Sequence
  Oct4dsRNA 111(21mer)
auaaaaaaacuaacagggctt

SEQ ID NO: 48 - DNA, 21 bp, Artificial Sequence
  Oct4dsRNA 111(21mer)
gcccuguuaguuuuuuuautt

SEQ ID NO: 49 - DNA, 13488 bp, Artificial Sequence
  pBacMam1 EBNA/OriP/Hyg DEST
ctcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaatctgcagaattcggcttaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatcctctagattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtaagccgaattccagcacactggcggccgttactagtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggcta

SEQ ID NO: 50 - DNA, 15 bp, Artificial Sequence
  Synthetic Sequence
gcttttttatactaa